(19) United States
(12) Patent Application Publication
(10) Pub. No.: US 2004/0006035 A1
(43) Pub. Date: Jan. 8, 2004

(54) Nucleic acid mediated disruption of HIV fusogenic peptide interactions

(76) Inventors: Dennis Macejak, Arvada, CO (US); Lawrence Blatt, San Francisco, CA (US); James McSwiggen, Boulder, CO (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/420,194

(22) Filed: Apr. 22, 2003

Related U.S. Application Data
(63) Continuation-in-part of application No. PCT/US03/05190, filed on Feb. 20, 2003.
(63) Continuation-in-part of application No. 10/225,023, filed on Aug. 21, 2002.
Application No. 10/225,023 is a continuation-in-part of application No. 10/157,580, filed on May 29, 2002.
(60) Provisional application No. 60/398,036, filed on Jul. 23, 2002.
(60) Provisional application No. 60/374,722, filed on Apr. 23, 2002.
(60) Provisional application No. 60/358,580, filed on Feb. 20, 2002.
(60) Provisional application No. 60/363,124, filed on Mar. 11, 2002.
(60) Provisional application No. 60/386,782, filed on Jun. 6, 2002.
(60) Provisional application No. 60/406,784, filed on Aug. 29, 2002.
(60) Provisional application No. 60/408,378, filed on Sep. 5, 2002.
(60) Provisional application No. 60/409,293, filed on Sep. 9, 2002.
(60) Provisional application No. 60/440,129, filed on Jan. 15, 2003.
(60) Provisional application No. 60/294,140, filed on May 29, 2001.

(51) Int. Cl. A61K 48/00; C07H 21/02

(52) U.S. Cl. 514/44; 536/23.72

(57) ABSTRACT

The present invention relates to nucleic acid aptamers that bind to HIV envelope glycoprotein, gp120 and/or gp41 and methods for their use alone or in combination with other therapies, such as HIV RT inhibitors and HIV protease inhibitors. Also disclosed are nucleic acids such as siRNA, antisense, and enzymatic nucleic acid molecules that can modulate the expression of HIV env genes and HIV viral replication. The compounds and methods of the invention are expected to inhibit HIV viral fusion, cell entry, gene expression, and replication.

This application is a continuation-in-part of PCT/US03/05190 filed Feb. 20, 2003, which claims the benefit of McSwiggen et al., U.S. Provisional Application Serial No. 60/398,036, filed Jul. 23, 2002, of McSwiggen U.S. Provisional Application Serial No. 60/374,722, filed Apr. 22, 2002, of Beigelman U.S. Provisional Application Serial No. 60/358,580, filed Feb. 20, 2002, of Beigelman U.S. Provisional Application Serial No. 60/363,124, filed Mar. 11, 2002, of Beigelman U.S. Provisional Application Serial No. 60/386,782, filed Jun. 6, 2002, of Beigelman U.S. Provisional Application Serial No. 60/406,784, filed Aug. 29, 2002, of Beigelman U.S. Provisional Application Serial No. 60/408,378, filed Sep. 5, 2002, of Beigelman U.S. Provisional Application Serial No. 60/409,293, filed Sep. 9, 2002, and of Beigelman U.S. Provisional Application Serial No. 60/440,129, filed Jan. 15, 2003 and which is a continuation-in-part of McSwiggen et al., U.S. patent application Ser. No. 10/225,023, filed Aug. 21, 2002, which is a continuation-in-part of McSwiggen et al., U.S. patent application Ser. No. 10/157,580, filed May 29, 2002, which claims the benefit of McSwiggen U.S. Provisional Application Serial No. 60/294,140, filed May 29, 2001. These applications are hereby incorporated by reference herein in their entireties, including the drawings.

### BACKGROUND OF THE INVENTION

The present invention concerns compounds, compositions, and methods for the study, diagnosis, and treatment of degenerative and disease states related to Human Immunodeficiency Virus (HIV) infection and/or acquired immunodeficiency syndrome (AIDS). Specifically, the invention relates to nucleic acid molecules used to inhibit HIV cell fusion and entry via disruption of fusogenic peptide interactions.

Human immunodeficiency virus type I (HIV-1) enters permissive cells by binding to the cellular receptor, CD4, followed by fusion of the viral and target cell membranes. Fusion results in viral entry into the target cell followed by integration and expression of the HIV-1 genome. The HIV-1 envelope glycoprotein mediates the fusion process through interaction with cellular receptors. The HIV-1 envelope glycoprotein is synthesized as a precursor protein, gp160, which is proteolytically processed to generate two subunits, the surface glycoprotein gp120 and the transmembrane glycoprotein gp41. These subunits remain noncovalently associated to form the oligomeric envelope glycoprotein spike on the viral membrane. Portions of gp120 bind to the CD4 receptor and a chemokine receptor (typically CCR5 or CXCR4) on the surface of target cells. These events trigger gp41 to undergo conformational changes that promote fusion of viral and cellular membranes, resulting in entry of the viral core into the cell. By analogy with the pH-induced structural changes in the hemagglutinin (HA) protein of influenza virus, the HIV-1 fusion activation process likely involves substantial conformational changes from a pre-fusogenic state to a fusogenic conformation.

The structure of the ectodomain of both HIV and SfV gp41 in the fusogenic/post-fusogenic state has been characterized by NMR and crystallography. These studies have shown that gp41 consists of a trimer of hairpins. In the fusogenic conformation of gp41, three N-terminal helices form a trimeric coiled coil, and three C-terminal helices pack in the reverse direction into three hydrophobic grooves on the surface of the coiled coil, bringing the amino and carboxy termini of the ectodomain together. Because the membrane anchor and the fusion peptide of the gp41 ectodomain are embedded in the viral and target cell membranes respectively, the formation of a fusogenic hairpin structure results in the colocalization of the two membranes. Peptides corresponding to the C-terminal region, referred to as C peptides, can specifically inhibit viral entry into target cells at nanomolar concentrations. One such peptide (T-20) is in clinical study and has shown antiviral activity in humans. T-20 binds to gp41 only after interaction of the envelope glycoprotein complex with the cellular receptors.

Jeffs et al., International PCT Publication No. WO 01/51673, describes isolated portions of gp41 protein (DP107 and DP178 domains) that are used to inhibit interaction between gp41 and gp120 and prevent infectivity of HIV.

Soukchareun et al., 1998, Bioconjugate Chemistry, 9, 466-475, describes the use of N-Fmoc-cysteine(S-thiobutyl) derivatized oligodeoxynucleotides for the preparation of certain gp41 peptide hybrid oligonucleotides having membranotropic activities.

### SUMMARY OF THE INVENTION

The present invention relates to nucleic acid molecules used to inhibit HIV cell fusion and entry via disruption of fusogenic peptide interactions. The invention also relates to nucleic acid molecules directed to disrupt the function of the HIV-1 envelope glycoprotein, such as to inhibit CD4 receptor mediated fusion of HIV-1. In particular, the present invention describes the selection and function of nucleic acid molecules, such as aptamers, capable of specifically binding to the HIV-1 envelope glycoprotein and modulating activity of the HIV-1 envelope glycoprotein or components thereof. These nucleic acid molecules can be used to treat diseases and disorders associated with HIV infection, or as a prophylactic measure to prevent HIV-1 infection.

The nucleic acid aptamers of the invention can be used as antifusogenic and antiviral agents. The antifusogenic activity of the aptamers of the invention can result from the ability to modulate intracellular processes that involve coiled-coil peptide structures or protein-protein interactions. The antiviral activity of the aptamers of the invention includes but is not limited to the inhibition of HIV transmission to uninfected CD-4⁺ cells.

The present invention also features the use of one or more nucleic acid-based techniques for modulating gene expression, such as nucleic acid aptamers, enzymatic nucleic acid molecules, small interfering RNA (siRNA), nucleic acid sensor molecules, allozymes, antisense nucleic acid molecules, 2,5-A nucleic acid chimeras, triplex oligonucleotides, and antisense nucleic acid molecules with nucleic acid cleaving groups, to modulate the activity, expression, or level of cellular proteins required for HIV cell fusion and entry. For example, the invention features the use of nucleic acid-based techniques to specifically modulate the activity and/or expression of proteins required for HIV cell fusion and entry.

In one embodiment, the invention features antifusogenic nucleic acid aptamers directed to disrupt the function of HIV-1 envelope glycoprotein or components thereof and prevent viral membrane fusion and/or entry. The nucleic acid aptamers of the invention are designed to interact with subunits of the HIV-1 envelope glycoprotein, such as the gp120 and gp41 subunits of the HIV-1 envelope glycoprotein, and disrupt the function of the HIV-1 envelope glycoprotein or components thereof. Such disruption of the HIV-1 envelope glycoprotein can be effected, for example, by preventing conformational changes to gp120 or gp41, and/or preventing protein-protein interactions between gp120 and/or gp41 or interactions within gp120 and/or gp41.

In another embodiment, the invention features antifusogenic nucleic acid aptamers having binding affinity to gp41. Non-limiting examples of target regions within the gp41 peptide sequence include sequences derived from the C-terminal region of gp41. For example, the present invention features aptamers having binding affinity to a peptide sequence corresponding to amino acids 638 to 673 of GP-41, and aptamers having binding affinity to a peptide sequence corresponding to amino acids 558 to 595 of GP-41 (see for example Jeffs et al., U.S. patent application Ser. No. 09/350,841, incorporated by reference herein in its entirety including the drawings).

In yet another embodiment, the invention features antifusogenic nucleic acid aptamers having binding affinity to peptide sequences having SEQ ID No. 1233 and/or SEQ ID No. 1234 (Table XII) or functional equivalents thereof. For example, in certain embodiments, nucleic acid aptamers of the invention can have binding affinity to analogs of the peptides contemplated herein, such analogs can contain one or more amino acid truncations, deletions, insertions or substitutions.

In one embodiment, the invention features an antifusogenic nucleic acid aptamer that specifically binds the HIV-1 envelope glycoprotein. In one embodiment, the invention features a nucleic acid aptamer that specifically binds the gp41 region of the HIV-1 envelope glycoprotein. In another embodiment, the invention features a nucleic acid aptamer molecule that specifically binds to the gp120 region of the HIV-1 envelope glycoprotein.

In one embodiment, nucleic acid aptamers of the invention act extracellularly and bind to their HIV-1 envelope glycoprotein targets outside of cells. Theses nucleic acid aptamers provide an attractive approach to treating HIV infection because they are able to act outside of cells or extracellularly.

In another embodiment, the invention features a composition comprising a nucleic acid aptamer of the invention in a pharmaceutically acceptable carrier. In another embodiment, the invention features a mammalian cell, for example a human cell, comprising a nucleic acid aptamer contemplated by the invention.

In one embodiment, the invention features a method for treatment of HIV-1 infection and/or AIDS, comprising administering to a patient a nucleic acid aptamer of the invention under conditions suitable for the treatment.

In another embodiment, the invention features a method of treatment of a patient having a condition associated with HIV-1 infection, comprising contacting cells of said patient with a nucleic acid aptamer of the invention under conditions suitable for such treatment. In another embodiment, the invention features a method of treatment of a patient having a condition associated with HIV-1 infection, comprising contacting cells of said patient with a nucleic acid aptamer of the invention, and further comprising the use of one or more drug therapies under conditions suitable for said treatment. Examples of suitable drug therapies include reverse transcriptase inhibitors such as zidovudine (AZT), zalcitabine (DDC), zidovudine (ZDV), lamivudine (3TC), didanosinedelavirdine (DDI), stavudine (D4T), abacavir, efavirenz, nevirapine, or tenofovir disoproxil fumarate, ribavirin and/or protease inhibitors such as indinavir, amprenavir, saquinavir, lopinavir, ritonavir, or nelfinavir, or any combination thereof. In another embodiment, the other therapy is administered simultaneously with or separately from the nucleic acid molecule.

In another embodiment, the invention features a method for modulating HIV cell fusion in a mammalian cell comprising administering to the cell a nucleic acid molecule of the invention under conditions suitable for the modulation.

In yet another embodiment, the invention features a method of modulating HIV cell fusion, comprising contacting a nucleic acid aptamer of the invention with HIV-1 envelope glycoprotein, gp120 and/or gp41 under conditions suitable for the modulating of the HIV cell fusion activity.

In one embodiment, a nucleic acid molecule of the invention, for example an aptamer or enzymatic nucleic acid molecule, is chemically synthesized. In another embodiment, the nucleic acid molecule of the invention comprises at least one nucleic acid sugar modification. In yet another embodiment, the nucleic acid molecule of the invention comprises at least one nucleic acid base modification. In another embodiment, the nucleic acid molecule of the invention comprises at least one nucleic acid backbone modification.

In one embodiment, the nucleic acid molecule of the invention comprises one or more ribonucleotides. In another embodiment, the nucleic acid molecule of the invention comprises one or more deoxy ribonucleotides.

In another embodiment, the nucleic acid molecule of the invention comprises at least one 2′-O-alkyl, 2′-alkyl, 2′-alkoxylalkyl, 2′-alkylthioalkyl, 2′-amino, 2′-O-amino, or 2′-halo modification and/or any combination thereof with or without 2′-deoxy and/or 2′-ribo nucleotides. In yet another embodiment, the nucleic acid molecule of the invention comprises all 2′-O-alkyl nucleotides, for example, all 2′-O-allyl nucleotides.

In one embodiment, the nucleic acid molecule of the invention comprises a 5′-cap, 3′-cap, or 5′-3′ cap structure, for example, an abasic or inverted abasic moiety.

In another embodiment, the nucleic acid molecule of the invention is a linear nucleic acid molecule. In another embodiment, the nucleic acid molecule of the invention is a linear nucleic acid molecule that can optionally form a hairpin, loop, stem-loop, or other secondary structure. In yet another embodiment, the nucleic acid molecule of the invention is a circular nucleic acid molecule.

In one embodiment, the nucleic acid molecule of the invention is a single stranded oligonucleotide. In another embodiment, the nucleic acid molecule of the invention is a double-stranded oligonucleotide.

In one embodiment, the nucleic acid molecule of the invention comprises an oligonucleotide having about 3 to about 500 nucleotides. In another embodiment, the nucleic acid molecule of the invention comprises an oligonucleotide having about 3 to about 24 nucleotides. In another embodiment, the nucleic acid molecule of the invention comprises an oligonucleotide having about 4 to about 16 nucleotides.

In one embodiment, the nucleic acid aptamer of the invention binds to its corresponding HIV-1 envelope derived target, with a binding affinity of about 100 μM-100 nM or about 20 to 50 nM, for example, by non-covalent interaction of the nucleic acid aptamer with a gp41 or gp120 derived peptide sequence, secondary or tertiary structure. In another embodiment, the nucleic acid aptamer of the invention binds to the HIV-1 envelope glycoprotein target with a binding affinity of less than about 20 nM.

In another embodiment, the nucleic acid aptamer of the invention binds irreversibly to the HIV-1 envelope derived target, for example, by covalent attachment of the nucleic acid aptamer to gp41 or gp120, or a gp4l or gp120 derived peptide sequence, secondary or tertiary structure. The covalent attachment can be accomplished by introducing chemical modifications into the nucleic acid aptamer's sequence that are capable of forming covalent bonds to the HIV-1 envelope glycoprotein target sequence.

In one embodiment, the invention features a composition comprising at least one HIV reverse transcriptase inhibitor and a nucleic acid molecule of the invention in a pharmaceutically acceptable carrier. In another embodiment, the invention features a composition comprising at least one HIV protease inhibitor and a nucleic acid molecule of the invention in a pharmaceutically acceptable carrier. In yet another embodiment, the invention features a composition comprising at least one HIV reverse transcriptase inhibitor, at least one HIV protease inhibitor and a nucleic acid molecule of the invention in a pharmaceutically acceptable carrier.

In another embodiment, the invention features a method of administering to a cell, for example a mammalian cell or human cell, a nucleic acid molecule of the invention independently or in conjunction with other therapeutic compounds such as HIV reverse transcriptase inhibitors and/or HIV protease inhibitors, comprising contacting the cell with the nucleic acid molecule and the HIV reverse transcriptase inhibitors and/or HIV protease inhibitors under conditions suitable for the administration.

In yet another embodiment, the invention features a method of administering to a cell, for example, a mammalian cell or human cell, a nucleic acid molecule of the invention independently or in conjunction with other therapeutic compounds, such as enzymatic nucleic acid molecules, antisense molecules, triplex forming oligonucleotides, 2,5-A chimeras, and/or RNAi molecules, comprising contacting the cell with the nucleic acid molecule of the invention under conditions suitable for the administration.

In another embodiment, administration of a nucleic acid molecule of the invention is administered to a cell or patient in the presence of a delivery reagent, for example a lipid, cationic lipid, phospholipid, or liposome.

In one embodiment, the invention features a method for identifying nucleic acid aptamers having HIV anti-fusogenic properties comprising: (a) generating a randomized pool of oligonucleotides; (b) combining the oligonucleotides from (a) with gp41 in vitro under conditions suitable to allow at least one oligonucleotide to bind to the target gp41 peptide; (c) removing non-bound oligonucleotide sequences from (b) under conditions suitable for isolating oligonucleotide sequences from (b) that possess binding affinity to gp41 by removing non-bound oligonucleotide sequences; (d) amplifying the oligonucleotide sequences isolated from (c) under conditions suitable for introducing some degree of mutation into the sequences; and (e) repeating steps (c) and (d) under conditions suitable for isolating one or more nucleic acid aptamers having binding affinity to gp41.

In another embodiment, the invention features a method for identifying nucleic acid aptamers having HIV anti-fusogenic properties comprising: (a) generating a randomized pool of oligonucleotides; (b) combining the oligonucleotides from (a) with gp120 in vitro under conditions suitable to allow at least one oligonucleotide to bind to the target gp120 peptide; (c)isolating oligonucleotide sequences from (b) that possess binding affinity to gp120 by removing non-bound oligonucleotide sequences; (d) amplifying the oligonucleotide sequences isolated from (c) under conditions suitable for introducing some degree of mutation into the sequences; and (e) repeating steps (c) and (d) under conditions suitable for isolating one or more nucleic acid aptamers having binding affinity to gp210.

In one embodiment, the invention features a method for identifying nucleic acid aptamers having HIV anti-fusogenic properties comprising: (a) generating a randomized pool of oligonucleotides; (b) combining the oligonucleotides from (a) with a target peptide derived from the HIV envelope glycoprotein in vitro under conditions suitable to allow at least one oligonucleotide to bind to the target peptide; (c) isolating oligonucleotide sequences from (b) that possess binding affinity to the target peptide by removing non-bound oligonucleotide sequences; (d) amplifying the oligonucleotide sequences isolated from (c) under conditions suitable for introducing some degree of mutation into the sequences; and (e) repeating steps (c) and (d) under conditions suitable for isolating one or more nucleic acid aptamers having binding affinity to the target peptide. In the described methods, the random pool of oligonucleotides can comprise DNA and/or RNA, with or without chemically modified nucleotides. When chemically modified nucleotides are used in the method, such modifications can be chosen such that a non-discriminatory polymerase will incorporate the chemically modified nucleotide into the oligonucleotide sequence when generated or amplified. Non-limiting examples of chemically modified nucleoside triphosphates (NTPs) that can be used in the method of the invention include 2′-deoxy-2′-fluoro, 2′-deoxy-2′-amino, 2′-O-alkyl, and 2′-O-methyl NTPs as well as various base modified NTPs, such as C5-modified pyrimidines, 2,6-diaminopurine, and inosine. The oligonucleotides used in the method can be of fixed or variable length. The target peptide derived from HIV envelope glycoprotein used in the method of the invention can comprise a synthetic or naturally occurring peptide that is synthesized or isolated from viral protein, for example by proteolytic cleavage. The target peptide can comprise sequence derived from proteins having sequence identical or similar to GenBank Accession Nos. AAM09869-AAM09880 or analogs thereof. For example, the target peptide can comprise sequences derived from gp41 or gp120 that are essential for HIV membrane fusion and viral entry activity, such as SEQ ID NOs. 1233 and/or 1234, and analogs thereof. These analogs can contain one or more amino acid truncations, deletions, insertions or substitutions. The conditions used in the method preferably provide nucleic acid aptamers that bind to their respective target in the conformation that the target adopts in its natural state. For example, peptide targets and binding conditions are chosen such that the isolated aptamer binds to its target site within the HIV envelope glycoprotein such that fusogenic activity of the protein is disrupted, such as by preventing intermolecular or intramolecular protein-protein interactions. The nucleic acid aptamers thus isolated by methods of the invention can be tested, for example, for an ability to inhibit cell fusion or viral activity using assays described herein.

In another embodiment, the method for identifying nucleic acid aptamers having HIV anti-fusogenic properties comprises attaching the target protein or peptide sequence to a solid matrix, such as beads, microtiter plate wells, membranes, or chip surfaces. In such a system, the target protein/peptide can be attached to the solid matrix either covalently or non-covalently. In yet another embodiment, the oligonucleotide or nucleic acid aptamer used in a method of the invention can be labeled, either directly or non-directly, for example with a radioactive label, absorption label such as biotin, or a fluorescent label such as fluorescein or rhodamine.

In one embodiment, the invention features novel nucleic acid-based techniques such as nucleic acid aptamers, used alone or in combination with enzymatic nucleic acid molecules, antisense molecules, and/or RNAi molecules, and methods for use to prevent HIV cellular fusion and entry or to down regulate or modulate the expression of HIV RNA and/or replication of HIV.

In another embodiment, the invention features the use of one or more nucleic acid-based techniques, such as nucleic acid aptamers, enzymatic nucleic acid molecules, small interfering RNA (siRNA), nucleic acid sensor molecules, allozymes, antisense nucleic acid molecules, 2,5-A nucleic acid chimeras, triplex oligonucleotides, and antisense nucleic acid molecules with nucleic acid cleaving groups, to modulate the activity, expression, or level of cellular proteins required for HIV cell fusion and entry. For example, the invention features the use of nucleic acid-based techniques to specifically modulate the activity and/or expression of proteins required for HIV cell fusion and entry, such as cellular receptors, cell surface molecules, cellular enzymes, cellular transcription factors, and/or cytokines, second messengers, and cellular accessory molecules.

Examples of such cellular receptors involved in HIV infection contemplated by the instant invention include, but are not limited to, CD4 receptors, CXCR4 (also known as Fusin; LESTR; NPY3R, e.g., Genbank Accession No. NM_{—}003467); CCR5 (also known as CKR-5, CMKRB5, e.g., Genbank Accession No. NM_{—}000579); CCR3 (also known as CC-CKR-3, CKR-3, CMKBR3, e.g., Genbank Accession No. NM_{—}001837); CCR2 (also known as CCR2b, CMKBR2, e.g., Genbank Accession Nos. NM_{—}000647 and NM_{—}000648); CCR1 (also known as CKR1, CMKBR1, e.g., Genbank Accession No. NM_{—}001295); CCR4 (also known as CKR-4, e.g., Genbank Accession No. NM_{—}005508); CCR8 (also known as ChemR1, TER1, CMKBR8, e.g., Genbank Accession No. NM_{—}005201); CCR9 (also known as D6, e.g. Genbank Accession Nos. NM_{—}006641 and NM_{—}031200); CXCR2 (also known as IL-8RB, e.g., Genbank Accession No. NM_{—}001557); STRL33 (also known as Bonzo; TYMSTR, e.g., Genbank Accession No. NM_{—}006564); US28; V28 (also known as CMKBRL1, CX3CR1, GPR13, e.g., Genbank Accession No. NM_{—}001337); gpr1 (also known as GPR1, e.g., Genbank Accession No. NM_{—}005279); gpr15 (also known as BOB, GPR15, e.g., Genbank Accession No. NM_{—}005290); Apj (also known as angiotensin-receptor-like, AGTRL1, e.g., Genbank Accession No. NM_{—}005161); and ChemR23 receptors (e.g., Genbank Accession No. NM_{—}004072).

Examples of cell surface molecules involved in HIV infection contemplated by the instant invention include, but are not limited to, Heparan Sulfate Proteoglycans, HSPG2 (e.g., Genbank Accession No. NM_{—}005529); SDC2 (e.g., Genbank Accession Nos. AK025488, J04621, J04621); SDC4 (e.g., Genbank Accession No. NM_{—}002999); GPC1 (e.g., Genbank Accession No. NM_{—}002081); SDC3 (e.g., Genbank Accession No. NM_{—}014654); SDC1 (e.g., Genbank Accession No. NM_{—}002997); Galactoceramides (e.g., Genbank Accession Nos. NM_{—}000153, NM_{—}003360, NM_{—}001478.2, NM_{—}004775, and NM_{—}004861); and Erythrocyte-expressed Glycolipids (e.g., Genbank Accession Nos. NM_{—}003778, NM_{—}003779, NM_{—}003780, NM_{—}030587, and NM_{—}001497).

Examples of cellular enzymes involved in HIV infection contemplated by the invention include, but are not limited to, N-myristoyltransferase (NMT1, e.g., Genbank Accession No. NM_{—}021079 and NMT2, e.g., Genbank Accession No. NM_{—}004808); Glycosylation Enzymes (e.g., Genbank Accession Nos. NM_{—}000303, NM_{—}013339, NM_{—}003358, NM_{—}005787, NM_{—}002408, NM_{—}002676, NM_{—}002435), NM_{—}002409, NM_{—}006122, NM_{—}002372, NM 006699, NM_{—}005907, NM_{—}004479, NM_{—}000150, NM_{—}005216 and NM_{—}005668); gp-160 Processing Enzymes (such as PCSK5, e.g., Genbank Accession No. NM_{—}006200); Ribonucleotide Reductase (e.g., Genbank Accession Nos. NM_{—}001034, NM_{—}001033, AB036063, AB036063, AB036532, AK001965, AK001965, AK023605, AL137348, and AL137348); and Polyamine Biosynthesis enzymes (e.g., Genbank Accession Nos. NM_{—}002539, NM_{—}003132 and NM_{—}001634).

Examples of cellular transcription factors involved in HIV infection contemplated by the invention include, but are not limited to, SP-1 and NF-kappa B (such as NFKB2, e.g., Genbank Accession No. NM_{—}002502; RELA, e.g., Genbank Accession No. NM_{—}021975; and NFKB1, e.g., Genbank Accession No. NM_{—}003998).

Examples of cytokines and second messengers involved in HIV infection contemplated by the invention include, but are not limited to, Tumor Necrosis Factor-a (TNF-a, e.g., Genbank Accession No. NM_{—}000594); Interleukin 1a (IL-1a, e.g., Genbank Accession No. NM_{—}000575); Interleukin 6 (IL-6, e.g., Genbank Accession No. NM_{—}000600); Phospholipase C (PLC, e.g., Genbank Accession No. NM_{—}000933); and Protein Kinase C (PKC, e.g., Genbank Accession No. NM_{—}006255).

Examples of cellular accessory molecules involved in HIV infection contemplated by the invention include, but are not limited to, Cyclophilins, (such as PPID, e.g., Genbank Accession No. NM_{—}005038; PPIA, e.g., Genbank Accession No. NM_{—}021130; PPIE, e.g., Genbank Accession No. NM_{—}006112; PPIB, e.g., Genbank Accession No. NM_{—}000942; PPIF, e.g., Genbank Accession No. NM 005729; PPIG, e.g., Genbank Accession No. NM_{—}004792; and PPIC, e.g., Genbank Accession No. NM_{—}000943); Mitogen Activated Protein Kinase (MAP-Kinase, such as MAPK1, e.g., Genbank Accession Nos. NM_{—}002745 and NM_{—}138957); and Extracellular Signal-Regulated Kinase (ERK-Kinase). In one embodiment, nucleic acid molecules of the invention are used to treat HIV-infected cells or a HIV-infected patient wherein the HIV is resistant or the patient does not respond to treatment with current antiviral therapeutics such as HIV reverse transcriptase or HIV protease inhibitors, either alone or in combination with other therapies under conditions suitable for the treatment.

The present invention also features nucleic acid molecules capable of modulating gene expression, such as enzymatic nucleic acid molecules, small interfering RNA (siRNA), nucleic acid sensor molecules, allozymes, antisense nucleic acid molecules, 2,5-A nucleic acid chimeras, triplex oligonucleotides, and antisense nucleic acid molecules with nucleic acid cleaving groups, which down regulate expression of a sequence encoding a human immunodeficiency virus (such as HIV-1, HIV-2, and related viruses such as FIV-1 and SIV-1) envelope glycoprotein gene (env), for example Genbank accession number NC_{—}001802 and/or sequences referred to in Table I. The sequence descriptions in Table I refer to composite names consisting of the following four parts: (a) HIV subtype (A, B, C, etc.); (b) Country of origin (US, JP, etc.); (c) Sampling year (2 digits, a “-” means the sampling year isn't entered); and (d) Sequence name or isolate name.

The present invention features an enzymatic nucleic acid molecule comprising SEQ ID NOs. 505-905. The invention also features an enzymatic nucleic acid molecule comprising at least one binding arm wherein one or more of said binding arms comprises a sequence complementary to any of SEQ ID NOs. 1-395.

In one embodiment, an enzymatic nucleic acid molecule of the invention is adapted to HIV infection or acquired immunodeficiency syndrome (AIDS).

In another embodiment, the enzymatic nucleic acid molecule of the invention has an endonuclease activity to cleave RNA having HIV env sequence.

In one embodiment, the enzymatic nucleic acid molecule of the invention is in an Inozyme, Zinzyme, G-cleaver, Amberzyme, DNAzyme Hairpin or Hammerhead configuration.

In one embodiment, an enzymatic nucleic acid molecule of the invention comprises between 12 and 100 bases complementary to a RNA sequence encoding HIV env. In another embodiment, an enzymatic nucleic acid molecule of the invention comprises between 14 and 24 bases complementary to a RNA sequence encoding HIV env.

In one embodiment, the Hammerhead of the invention comprises a sequence selected from the group consisting of SEQ ID NOs 505-561.

In one embodiment, the Inozyme of the invention comprises a sequence selected from the group consisting of SEQ ID NOs. 562-637.

In one embodiment, the G-cleaver of the invention comprises a sequence selected from the group consisting of SEQ ID NOs. 638-661. In one embodiment, the Zinzyme of the invention comprises a sequence selected from the group consisting of SEQ ID NOs. 662-705.

In one embodiment, the DNAzyme of the invention comprises a sequence selected from the group consisting of SEQ ID NOs. 706-806.

In one embodiment, the Amberzyme of the invention comprises a sequence selected from the group consisting of SEQ ID NOs 807-905.

In one embodiment, the antisense molecule of the invention comprises a sequence complementary to a sequence of SEQ ID NOs. 1-395. In another embodiment, the antisense molecule of the invention comprises a sequence selected from the group consisting of SEQ ID Nos. 906-1014.

In one embodiment, the siRNA molecule of the invention comprises a sequence complementary to a sequence of SEQ ID NOs. 1-395. In another embodiment, the siRNA molecule of the invention comprises a duplex of sequences selected from the group consisting of SEQ ID Nos. 1015-1232.

In another embodiment, a nucleic acid molecule of the invention is chemically synthesized. A nucleic acid molecule of the invention can comprise at least one 2′-sugar modification, at least one nucleic acid base modification, and/or at least one phosphate backbone modification.

In one embodiment the present invention features a mammalian cell comprising a nucleic acid molecule of the invention. In one embodiment, the mammalian cell of the invention is a human cell.

The invention features a method of reducing HIV activity in a cell comprising contacting the cell with a nucleic acid molecule of the invention under conditions suitable for the reduction of HIV activity.

The invention also features a method of treating a patient having a condition associated with the level of HIV comprising contacting cells of the patient with a nucleic acid molecule of the invention under conditions suitable for the treatment.

In one embodiment, methods of treatment contemplated by the invention comprise the use of one or more drug therapies under conditions suitable for the treatment.

The invention features a method of cleaving RNA of a HIV env gene comprising contacting a nucleic acid molecule of the invention with the RNA of HIV env gene under conditions suitable for the cleavage. In one embodiment, the cleavage contemplated by the invention is carried out in the presence of a divalent cation, for example Mg2+.

In another embodiment, the nucleic acid molecule of the invention comprises a cap structure, wherein the cap structure is at the 5′-end, or 3′-end, or both the 5′-end and the 3′-end of the enzymatic nucleic acid molecule, for example, a 3′,3′-linked or 5′,5′-linked deoxyabasic ribose derivative.

The present invention features an expression vector comprising a nucleic acid sequence encoding at least one nucleic acid molecule of the invention in a manner which allows expression of the nucleic acid molecule.

The invention also features a mammalian cell, for example, a human cell comprising an expression vector contemplated by the invention.

In one embodiment, an expression vector of the invention comprises a nucleic acid sequence encoding two or more nucleic acid molecules, which may be the same or different.

The present invention features a method for treatment of acquired immunodeficiency syndrome (AIDS) or an AIDS related condition, for example Kaposi's sarcoma, lymphoma, cervical cancer, squamous cell carcinoma, cardiac myopathy, rheumatic disease, or opportunistic infection, comprising administering to a patient a nucleic acid molecule of the invention under conditions suitable for the treatment.

In one embodiment, a nucleic acid molecule of the invention comprises at least five ribose residues, at least ten 2′-O-methyl modifications, and a 3′-end modification, for example, a 3′-3′ inverted abasic moiety.

In another embodiment, a nucleic acid molecule of the invention further comprises phosphorothioate linkages on at least three of the 5′ terminal nucleotides.

In yet another embodiment, a DNAzyme of the invention comprises at least ten 2′-O-methyl modifications and a 3′-end modification, for example a 3′-3′ inverted abasic moiety. In a further embodiment, the DNAzyme of the invention further comprises phosphorothioate linkages on at least three of the 5′ terminal nucleotides.

In another embodiment, other drug therapies of the invention comprise antiviral therapy, monoclonal antibody therapy, chemotherapy, radiation therapy, analgesic therapy, or anti-inflammatory therapy.

In yet another embodiment, antiviral therapy of the invention comprises treatment with zidovudine (AZT), zalcitabine (DDC), zidovudine (ZDV), lamivudine (3TC), didanosinedelavirdine (DDI), stavudine (D4T), abacavir, efavirenz, nevirapine, or tenofovir disoproxil fumarate, ribavirin and/or protease inhibitors such as indinavir, amprenavir, saquinavir, lopinavir, ritonavir, or nelfinavir, or any combination thereof.

The invention features a composition comprising a nucleic acid molecule of the invention in a pharmaceutically acceptable carrier.

In one embodiment, the invention features a method of administering to a cell, for example a mammalian cell or human cell, a nucleic acid molecule of the invention comprising contacting the cell with the nucleic acid molecule under conditions suitable for the administration. The method of administration can be in the presence of a delivery reagent, for example, a lipid, cationic lipid, phospholipid, or liposome.

The term “antifusogenic” as used herein refers to the ability of a compound to inhibit or reduce the level of membrane fusion events between two or more moieties relative to the level of membrane fusion which occurs between the moieties in the absence of the compound. The moieties can be, for example, cell membranes or viral structures, such as viral envelopes or pili. Antifusogenic compounds can exert their effect by modulating protein-protein interactions or by modulating intracellular events involving coiled-coil peptide structures.

The term “antiviral” as used herein refers to the ability of a compound to inhibit or reduce viral infection of cells, for example, by inhibiting cell-cell fusion or free virus infection. The antiviral activity of the compound can result from antifusogenic activity or by preventing viral replication and/or expression, such as by modulating the expression of the viral genome.

The term “modulate” as used herein refers to a stimulatory or inhibitory effect on the intracellular or intercellular process of interest relative to the level or activity of such a process in the absence of a nucleic acid molecule of the invention. For example, the level of membrane fusion events between two or more moieties is enhanced or decreased in the presence of a modulator relative to the level of membrane fusion which occurs between the moieties in the absence of the modulator. In another non-limiting example, the expression of the gene, or level of RNA molecules or equivalent RNA molecules encoding one or more proteins or protein subunits, or activity of one or more proteins or protein subunits is up regulated or down regulated, such that expression, level, or activity is greater than or less than that observed in the absence of the modulator. For example, the term “modulate” can mean “inhibit,” but the use of the word “modulate” is not limited to this definition.

The term “inhibit” as used herein refers to when the activity of HIV envelope glycoprotein, or level of RNAs or equivalent RNAs encoding one or more protein subunits of HIV envelope glycoprotein or functional equivalents thereof, is reduced below that observed in the absence of the nucleic acid of the invention. In one embodiment, inhibition with nucleic acid molecule preferably is below that level observed in the presence of non-binding or an inactive or attenuated molecule that is unable to bind to the same target site. In another embodiment, inhibition of HIV gene expression, cell fusion or cell entry with the nucleic acid molecule of the instant invention is greater in the presence of the nucleic acid molecule than in its absence.

The methods of this invention can be used to treat HIV infections, which include productive virus infection, latent or persistent virus infection. The utility can be extended to other species of HIV that infect non-human animals where such infections are of veterinary importance.

By “aptamer” or “nucleic acid aptamer” as used herein is meant a nucleic acid molecule that binds specifically to a target molecule. The target molecule can be any molecule of interest. For example, the aptamer can be used to bind to a ligand-binding domain of a protein, thereby preventing interaction of the naturally occurring ligand with the protein. The aptamer can also be used to prevent protein-protein interactions or conformational changes within a protein by binding to a portion of a target protein that interacts with another protein or with another portion of the same protein. This is a non-limiting example and those in the art will recognize that other embodiments can be readily generated using techniques generally known in the art, see for example Gold et al., 1995, Annu. Rev. Biochem., 64, 763; Brody and Gold, 2000, J. Biotechnol., 74, 5; Sun, 2000, Curr. Opin. Mol. Ther., 2, 100; Kusser, 2000, J. Biotechnol., 74, 27; Hermann and Patel, 2000, Science, 287, 820; and Jayasena, 1999, Clinical Chemistry, 45, 1628.

By “enzymatic nucleic acid molecule” is meant a nucleic acid molecule that has complementarity in a substrate binding region to a specified gene target, and also has an enzymatic activity which is active to specifically cleave a target RNA or DNA molecule. That is, the enzymatic nucleic acid molecule is able to intermolecularly cleave a RNA or DNA molecule and thereby inactivate a target RNA or DNA molecule. These complementary regions allow sufficient hybridization of the enzymatic nucleic acid molecule to a target RNA molecule and thus permit cleavage. One hundred percent complementarity is preferred, but complementarity as low as 50-75% may also be useful in this invention (see for example Werner and Uhlenbeck, 1995, Nucleic Acids Research, 23, 2092-2096; Hammann et al., 1999, Antisense and Nucleic Acid Drug Dev., 9, 25-31). The nucleic acids can be modified at the base, sugar, and/or phosphate groups. The term enzymatic nucleic acid is used interchangeably with phrases such as ribozymes, catalytic RNA, enzymatic RNA, catalytic DNA, aptazyme or aptamer-binding ribozyme, regulatable ribozyme, catalytic oligonucleotides, nucleozyme, DNAzyme, RNA enzyme, endoribonuclease, endonuclease, minizyme, leadzyme, oligozyme or DNA enzyme. All of these terminologies describe nucleic acid molecules with enzymatic activity. The specific enzymatic nucleic acid molecules described in the instant application are not limiting in the invention and those skilled in the art will recognize that all that is important in an enzymatic nucleic acid molecule of this invention is that it have a specific substrate binding site which is complementary to one or more of the target nucleic acid regions, and that it have nucleotide sequences within or surrounding that substrate binding site which impart a nucleic acid cleaving activity to the molecule (Cech et al., U.S. Pat. No. 4,987,071; Cech et al., 1988, JAMA 260:20 3030-4).

By “nucleic acid molecule” as used herein is meant a molecule comprising nucleotides. The nucleic acid can be single, double, or multiple stranded and can comprise modified or unmodified nucleotides or non-nucleotides or various mixtures and combinations thereof.

By “Inozyme” or “NCH” motif or configuration is meant, an enzymatic nucleic acid molecule comprising a motif as is generally described as NCH Rz in Ludwig et al., International PCT Publication No. WO 98/58058 and U.S. patent application Ser. No. 08/878,640, which is herein incorporated by reference in its entirety including the drawings. Inozymes possess endonuclease activity to cleave RNA substrates having a cleavage triplet NCH/, where N is a nucleotide, C is cytidine and H is adenosine, uridine or cytidine, and / represents the cleavage site. Inozymes can also possess endonuclease activity to cleave RNA substrates having a cleavage triplet NCN/, where N is a nucleotide, C is cytidine, and / represents the cleavage site.

By “G-cleaver” motif or configuration is meant, an enzymatic nucleic acid molecule comprising a motif as is generally described in Eckstein et al., U.S. Pat. No. 6,127,173, which is herein incorporated by reference in its entirety including the drawings, and in Kore et al., 1998, Nucleic Acids Research 26, 4116-4120. G-cleavers possess endonuclease activity to cleave RNA substrates having a cleavage triplet NYN/, where N is a nucleotide, Y is uridine or cytidine and / represents the cleavage site. G-cleavers can be chemically modified.

By “zinzyme” motif or configuration is meant, an enzymatic nucleic acid molecule comprising a motif as is generally described in Beigelman et al., International PCT publication No. WO 99/55857 and U.S. patent application Ser. No. 09/918,728, which is herein incorporated by reference in its entirety including the drawings. Zinzymes possess endonuclease activity to cleave RNA substrates having a cleavage triplet including but not limited to, YG/Y, where Y is uridine or cytidine, and G is guanosine and / represents the cleavage site. Zinzymes can be chemically modified to increase nuclease stability through various substitutions, including substituting 2′-O-methyl guanosine nucleotides for guanosine nucleotides. In addition, differing nucleotide and/or non-nucleotide linkers can be used to substitute the 5′-gaaa-2′ loop of the motif. Zinzymes represent a non-limiting example of an enzymatic nucleic acid molecule that does not require a ribonucleotide (2′-OH) group within its own nucleic acid sequence for activity.

By “amberzyme” motif or configuration is meant, an enzymatic nucleic acid molecule comprising a motif as is generally described in Beigelman et al., International PCT publication No. WO 99/55857 and U.S. patent application Ser. No. 09/476,387, which is herein incorporated by reference in its entirety including the drawings. Amberzymes possess endonuclease activity to cleave RNA substrates having a cleavage triplet NG/N, where N is a nucleotide, G is guanosine, and / represents the cleavage site. Amberzymes can be chemically modified to increase nuclease stability. In addition, differing nucleoside and/or non-nucleoside linkers can be used to substitute the 5′-gaaa-3′ loops of the motif. Amberzymes represent a non-limiting example of an enzymatic nucleic acid molecule that does not require a ribonucleotide (2′-OH) group within its own nucleic acid sequence for activity.

By ‘DNAzyme’ is meant, an enzymatic nucleic acid molecule that does not require the presence of a 2′-OH group within its own nucleic acid sequence for activity. In particular embodiments, the enzymatic nucleic acid molecule can have an attached linker or linkers or other attached or associated groups, moieties, or chains containing one or more nucleotides with 2′-OH groups. DNAzymes can be synthesized chemically or expressed endogenously in vivo, by means of a single stranded DNA vector or equivalent thereof. Non-limiting examples of DNAzymes are generally reviewed in Usman et al., U.S. Pat. No. 6,159,714, which is herein incorporated by reference in its entirety including the drawings; Chartrand et al., 1995, NAR 23, 4092; Breaker et al., 1995, Chem. Bio. 2, 655; Santoro et al., 1997, PNAS 94, 4262; Breaker, 1999, Nature Biotechnology, 17, 422-423; and Santoro et. al., 2000, J. Am. Chem. Soc., 122, 2433-39. The “10-23” DNAzyme motif is one particular type of DNAzyme that was evolved using in vitro selection as generally described in Joyce et al., U.S. Pat. No. 5,807,718 and Santoro et al., supra. Additional DNAzyme motifs can be selected for using techniques similar to those described in these references, and hence, are within the scope of the present invention.

By “nucleic acid sensor molecule” or “allozyme” as used herein is meant a nucleic acid molecule comprising an enzymatic domain and a sensor domain, where the ability of the enzymatic nucleic acid domain's ability to catalyze a chemical reaction is dependent on the interaction with a target signaling molecule, such as a nucleic acid, polynucleotide, oligonucleotide, peptide, polypeptide, or protein, for example HIV-1 envelope glygoprotein, gp41, or gp120. The introduction of chemical modifications, additional functional groups, and/or linkers, to the nucleic acid sensor molecule can provide enhanced catalytic activity of the nucleic acid sensor molecule, increased binding affinity of the sensor domain to a target nucleic acid, and/or improved nuclease/chemical stability of the nucleic acid sensor molecule, and are hence within the scope of the present invention (see for example Usman et al., U.S. patent application Ser. No. 09/877,526, George et al., U.S. Pat. Nos. 5,834,186 and 5,741,679, Shih et al., U.S. Pat. No. 5,589,332, Nathan et al., U.S. Pat. No 5,871,914, Nathan and Ellington, International PCT publication No. WO 00/24931, Breaker et al., International PCT Publication Nos. WO 00/26226 and 98/27104, and Sullenger et al., U.S. patent application Ser. No. 09/205,520).

By “sensor component” or “sensor domain” of the nucleic acid sensor molecule as used herein is meant, a nucleic acid sequence (e.g., RNA or DNA or analogs thereof) which interacts with a target signaling molecule, for example a nucleic acid sequence in one or more regions of a target nucleic acid molecule or more than one target nucleic acid molecule, and which interaction causes the enzymatic nucleic acid component of the nucleic acid sensor molecule to either catalyze a reaction or stop catalyzing a reaction. In the presence of target signaling molecule of the invention, such as HIV-1 envelope glycoprotein or portions thereof such as gp41 and/or gp120, the ability of the sensor component, for example, to modulate the catalytic activity of the nucleic acid sensor molecule, is modulated or diminished. The sensor component can comprise recognition properties relating to chemical or physical signals capable of modulating the nucleic acid sensor molecule via chemical or physical changes to the structure of the nucleic acid sensor molecule. The sensor component can be derived from a naturally occurring nucleic acid binding sequence, for example, RNAs that bind to other nucleic acid sequences in vivo. Alternately, the sensor component can be derived from a nucleic acid molecule (aptamer), which is evolved to bind to a nucleic acid sequence within a target nucleic acid molecule. The sensor component can be covalently linked to the nucleic acid sensor molecule, or can be non-covalently associated. A person skilled in the art will recognize that all that is required is that the sensor component is able to selectively modulate the activity of the nucleic acid sensor molecule to catalyze a reaction.

By “target molecule” or “target signaling molecule” is meant a molecule capable of interacting with a nucleic acid sensor molecule, specifically a sensor domain of a nucleic acid sensor molecule, in a manner that causes the nucleic acid sensor molecule to be active or inactive. The interaction of the signaling agent with a nucleic acid sensor molecule can result in modification of the enzymatic nucleic acid component of the nucleic acid sensor molecule via chemical, physical, topological, or conformational changes to the structure of the molecule, such that the activity of the enzymatic nucleic acid component of the nucleic acid sensor molecule is modulated, for example is activated or deactivated. Signaling agents can comprise target signaling molecules such as macromolecules, ligands, small molecules, metals and ions, nucleic acid molecules including but not limited to RNA and DNA or analogs thereof, proteins, peptides, antibodies, polysaccharides, lipids, sugars, microbial or cellular metabolites, pharmaceuticals, and organic and inorganic molecules in a purified or unpurified form, for example HIV envelope glycoprotein or portions thereof such as gp41, gp120, and/or peptide sequences such as SEQ ID Nos 1233 and 1234 or analogs thereof.

By “sufficient length” is meant a nucleic acid molecule long enough to provide the intended function under the expected condition. For example, a nucleic acid molecule of the invention needs to be of “sufficient length” to provide stable binding to a target site under the expected binding conditions and environment. In another non-limiting example, for the binding arms of an enzymatic nucleic acid, “sufficient length” means that the binding arm sequence is long enough to provide stable binding to a target site under the expected reaction conditions and environment. The binding arms are not so long as to prevent useful turnover of the nucleic acid molecule. By “stably interact” is meant interaction of the oligonucleotides with target, such as a target protein or target nucleic acid (e.g., by forming hydrogen bonds with complementary amino acids or nucleotides in the target under physiological conditions) that is sufficient for the intended purpose (e.g., specific binding to a protein target to disrupt the function of that protein or cleavage of target RNA/DNA by an enzyme).

By “homology” is meant the nucleotide sequence of two or more nucleic acid molecules, or the amino acid sequence of two or more proteins, is partially or completely identical.

By “antisense nucleic acid”, it is meant a non-enzymatic nucleic acid molecule that binds to target RNA by means of RNA-RNA or RNA-DNA or RNA-PNA (protein nucleic acid; Egholm et al., 1993 Nature 365, 566) interactions and alters the activity of the target RNA (for a review, see Stein and Cheng, 1993 Science 261, 1004 and Woolf et al., U.S. Pat. No. 5,849,902). Typically, antisense molecules are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, the antisense molecule can be complementary to two or more non-contiguous substrate sequences or two or more non-contiguous sequence portions of an antisense molecule can be complementary to a target sequence, or both. For a review of current antisense strategies, see Schmajuk et al., 1999, J. Biol. Chem., 274, 21783-21789, Delihas et al., 1997, Nature, 15, 751-753, Stein et al., 1997, Antisense N. A. Drug Dev., 7, 151, Crooke, 2000, Methods Enzymol., 313, 3-45; Crooke, 1998, Biotech. Genet. Eng. Rev., 15, 121-157, Crooke, 1997, Ad. Pharmacol., 40, 1-49. Antisense molecules of the instant invention can include 2-5A antisense chimera molecules. In addition, antisense DNA can be used to target RNA by means of DNA-RNA interactions, thereby activating RNase H, which digests the target RNA in the duplex. The antisense oligonucleotides can comprise one or more RNAse H activating region that is capable of activating RNAse H cleavage of a target RNA. Antisense DNA can be synthesized chemically or expressed via the use of a single stranded DNA expression vector or equivalent thereof.

By “RNase H activating region” is meant a region (generally greater than or equal to 4-25 nucleotides in length, preferably from 5-11 nucleotides in length) of a nucleic acid molecule capable of binding to a target RNA to form a non-covalent complex that is recognized by cellular RNase H enzyme (see for example Arrow et al., U.S. Pat. No. 5,849,902; Arrow et al., U.S. Pat. No. 5,989,912). The RNase H enzyme binds to the nucleic acid molecule-target RNA complex and cleaves the target RNA sequence. The RNase H activating region comprises, for example, phosphodiester, phosphorothioate (for example, at least four of the nucleotides are phosphorothioate substitutions; more specifically, 4-11 of the nucleotides are phosphorothioate substitutions), phosphorodithioate, 5′-thiophosphate, or methylphosphonate backbone chemistry or a combination thereof. In addition to one or more backbone chemistries described above, the RNase H activating region can also comprise a variety of sugar chemistries. For example, the RNase H activating region can comprise deoxyribose, arabino, fluoroarabino or a combination thereof, nucleotide sugar chemistry. Those skilled in the art will recognize that the foregoing are non-limiting examples and that any combination of phosphate, sugar and base chemistry of a nucleic acid that supports the activity of RNase H enzyme is within the scope of the definition of the RNase H activating region and the instant invention.

By “2-5A antisense chimera” it is meant, an antisense oligonucleotide containing a 5′-phosphorylated 2′-5′-linked adenylate residue. These chimeras bind to target RNA in a sequence-specific manner and activate a cellular 2-5A-dependent ribonuclease, which, in turn, cleaves the target RNA (Torrence et al., 1993 Proc. Natl. Acad. Sci. USA 90, 1300).

By “triplex nucleic acid” or “triplex oligonucleotide” it is meant a polynucleotide or oligonucleotide that can bind to a double-stranded DNA in a sequence-specific manner to form a triple-strand helix. Formation of such triple helix structure has been shown to modulate transcription of the targeted gene (Duval-Valentin et al., 1992, Proc. Natl. Acad. Sci. USA, 89, 504). Triplex nucleic acid molecules of the invention also include steric blocker nucleic acid molecules that bind to the Enhancer I region of HBV DNA (plus strand and/or minus strand) and prevent translation of HBV genomic DNA.

The term “short interfering nucleic acid”, “siNA”, “short interfering RNA”, “siRNA”, “short interfering nucleic acid molecule”, “short interfering oligonucleotide molecule”, or “chemically-modified short interfering nucleic acid molecule” as used herein refers to any nucleic acid molecule capable of inhibiting or down regulating gene expression or viral replication, for example by mediating RNA interference “RNAi” or gene silencing in a sequence-specific manner; see for example Bass, 2001, Nature, 411, 428-429; Elbashir et al., 2001, Nature, 411, 494-498; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237; Hutvagner and Zamore, 2002, Science, 297, 2056-60; McManus et al., 2002, RNA, 8, 842-850; Reinhart et al., 2002, Gene & Dev., 16, 1616-1626; and Reinhart & Bartel, 2002, Science, 297, 1831). For example the siNA can be a double-stranded polynucleotide molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. The siNA can be assembled from two separate oligonucleotides, where one strand is the sense strand and the other is the antisense strand, wherein the antisense and sense strands are self-complementary (i.e. each strand comprises nucleotide sequence that is complementary to nucleotide sequence in the other strand; such as where the antisense strand and sense strand form a duplex or double stranded structure, for example wherein the double stranded region is about 19 base pairs); the antisense strand comprises nucleotide sequence that is complementary to nucleotide sequence in a target nucleic acid molecule or a portion thereof and the sense strand comprises nucleotide sequence corresponding to the target nucleic acid sequence or a portion thereof. Alternatively, the siNA is assembled from a single oligonucleotide, where the self-complementary sense and antisense regions of the siNA are linked by means of a nucleic acid based or non-nucleic acid-based linker(s). The siNA can be a polynucleotide with a hairpin secondary structure, having self-complementary sense and antisense regions, wherein the antisense region comprises nucleotide sequence that is complementary to nucleotide sequence in a separate target nucleic acid molecule or a portion thereof and the sense region having nucleotide sequence corresponding to the target nucleic acid sequence or a portion thereof. The siNA can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siNA molecule capable of mediating RNAi. The siNA can also comprise a single stranded polynucleotide having nucleotide sequence complementary to nucleotide sequence in a target nucleic acid molecule or a portion thereof (for example, where such siNA molecule does not require the presence within the siNA molecule of nucleotide sequence corresponding to the target nucleic acid sequence or a portion thereof), wherein the single stranded polynucleotide can further comprise a terminal phosphate group, such as a 5′-phosphate (see for example Martinez et al., 2002, Cell., 110, 563-574 and Schwarz et al., 2002, Molecular Cell, 10, 537-568), or 5′,3′-diphosphate. In certain embodiment, the siNA molecule of the invention comprises separate sense and antisense sequences or regions, wherein the sense and antisense regions are covalently linked by nucleotide or non-nucleotide linkers molecules as is known in the art, or are alternately non-covalently linked by ionic interactions, hydrogen bonding, van der waals interactions, hydrophobic intercations, and/or stacking interactions. In certain embodiments, the siNA molecules of the invention comprise nucleotide sequence that is complementary to nucleotide sequence of a target gene. In another embodiment, the siNA molecule of the invention interacts with nucleotide sequence of a target gene in a manner that causes inhibition of expression of the target gene. As used herein, siNA molecules need not be limited to those molecules containing only RNA, but further encompasses chemically-modified nucleotides and non-nucleotides. In certain embodiments, the short interfering nucleic acid molecules of the invention lack 2′-hydroxy (2′-OH) containing nucleotides. Applicant describes in certain embodiments short interfering nucleic acids that do not require the presence of nucleotides having a 2′-hydroxy group for mediating RNAi and as such, short interfering nucleic acid molecules of the invention optionally do not include any ribonucleotides (e.g., nucleotides having a 2′-OH group). Such siNA molecules that do not require the presence of ribonucleotides within the siNA molecule to support RNAi can however have an attached linker or linkers or other attached or associated groups, moieties, or chains containing one or more nucleotides with 2′-OH groups. Optionally, siNA molecules can comprise ribonucleotides at about 5, 10, 20, 30, 40, or 50% of the nucleotide positions. The modified short interfering nucleic acid molecules of the invention can also be referred to as short interfering modified oligonucleotides “siMON.” As used herein, the tern siNA is meant to be equivalent to other terms used to describe nucleic acid molecules that are capable of mediating sequence specific RNAi, for example short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), and others. In addition, as used herein, the term RNAi is meant to be equivalent to other terms used to describe sequence specific RNA interference, such as post transcriptional gene silencing, or epigenetics. For example, siNA molecules of the invention can be used to epigenetically silence genes at both the post-transcriptional level or the pre-transcriptional level. In a non-limiting example, epigenetic regulation of gene expression by siNA molecules of the invention can result from siNA mediated modification of chromatin structure to alter gene expression (see, for example, Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237).

By “gene” it is meant, a nucleic acid that encodes an RNA, for example, nucleic acid sequences including, but not limited to, structural genes encoding a polypeptide.

By “complementarity” is meant that a nucleic acid can form hydrogen bond(s) with another nucleic acid sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., ribozyme cleavage, antisense or triple helix modulation. Determination of binding free energies for nucleic acid molecules is well known in the art (see, e.g., Turner et al., 1987, CSH Symp. Quant. Biol. LII pp.123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

The nucleic acid aptamers that bind to a HIV envelope glycoprotein and therefore inactivate the cellular fusion and entry represent a novel therapeutic approach to treat HIV infection, AIDS and related conditions.

In one embodiment of the present invention, an aptamer nucleic acid molecule of the invention is about 4 to about 50 nucleotides in length, in specific embodiments about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In another embodiment, an enzymatic nucleic acid molecule of the invention, e.g., a ribozyme or DNAzyme, is about 13 to about 100 nucleotides in length, e.g., in specific embodiments about 22, 23, 24, 25, 26, 27, 28, 29, 30, 31, 32, 33, 34, 35, 36, 37, or 38 nucleotides in length. In another embodiment, an antisense nucleic acid molecule, 2,5-A chimera, or triplex oligonucleotide of the invention is about 13 to about 100 nucleotides in length, e.g., in specific embodiments about 22, 23, 24, 25, 26, 27, 28, 29, 30, 31, 32, 33, 34, 35, 36, 37, or 38 nucleotides in length. In another embodiment, a siRNA molecule of the invention is about 18 to about 24 nucleotides in length (such as where each strand of siRNA duplex is about 18 to about 24 nucleotides in length), e.g., in specific embodiments, each strand of the siRNA duplex is about 18, 19, 20, 21, 22, 23, or 24 nucleotides in length. In yet another embodiment, a siRNA molecule of the invention has 2 3′-nucleotide overhangs on each strand of the duplex, for example two thymidine (TT) nucleotide overhangs. In particular embodiments, instead of 100 nucleotides being the upper limit on the length ranges specified above, the upper limit of the length range can be, for example, 30, 40, 50, 60, 70, or 80 nucleotides. Thus, for any of the length ranges, the length range for particular embodiments has lower limit as specified, with an upper limit as specified which is greater than the lower limit. For example, in a particular embodiment, the length range can be 20-50 nucleotides in length. All such ranges are expressly included. Also in particular embodiments, a nucleic acid molecule can have a length which is any of the lengths specified above, for example, 21 nucleotides in length.

Aptamer molecules of the invention are about 4 to about 50 nucleotides in length. Exemplary siRNA molecules of the invention are about 18 to about 24 nucleotides in length for each strand of the siRNA duplex. In an additional example, enzymatic nucleic acid molecules of the invention are preferably about 15 to about 50 nucleotides in length, more preferably about 25 to about 40 nucleotides in length, e.g., 34, 36, or 38 nucleotides in length (for example see Jarvis et al., 1996, J. Biol. Chem., 271, 29107-29112). Exemplary DNAzymes of the invention are preferably about 15 to about 40 nucleotides in length. In one embodiment, exemplary DNAzymes are about 25 to about 35 nucleotides in length, e.g., 29, 30, 31, or 32 nucleotides in length (see for example Santoro et al., 1998, Biochemistry, 37, 13330-13342; Chartrand et al., 1995, Nucleic Acids Research, 23, 4092-4096). Exemplary antisense molecules of the invention are about 15 to about 75 nucleotides in length. In one embodiment, exemplary antisense molecules are about 20 to about 35 nucleotides in length, e.g., 25, 26, 27, or 28 nucleotides in length (see for example Woolf et al., 1992, PNAS., 89, 7305-7309; Milner et al., 1997, Nature Biotechnology, 15, 537-541). Exemplary triplex forming oligonucleotide molecules of the invention are about 10 to about 40 nucleotides in length. In one embodiment, exemplary triplex forming oligonucleotide molecules are about 12 to about 25 nucleotides in length, e.g., 18, 19, 20, or 21 nucleotides in length (see for example Maher et al., 1990, Biochemistry, 29, 8820-8826; Strobel and Dervan, 1990, Science, 249, 73-75). Those skilled in the art will recognize that all that is required is that the nucleic acid molecule is of length and conformation sufficient and suitable for the nucleic acid molecule to catalyze a reaction contemplated herein. The length of the nucleic acid molecules of the instant invention are not limiting within the general limits stated.

In one embodiment, the invention provides a method for producing a class of nucleic acid aptamers which exhibit a high degree of specificity for a HIV envelope glycoprotein such as a site within the gp41 region of HIV envelope glycoprotein. In another embodiment, the invention provides a method for producing a class of nucleic acid based gene modulating agents which exhibit a high degree of specificity for HIV nucleic acid sequences encoding the HIV envelope glycoprotein. For example, the nucleic acid gene modulating molecule is preferably targeted to a highly conserved region of the HIV env gene such that specific treatment of a disease or condition can be provided with either one or several nucleic acid molecules of the invention. Alternately, the nucleic acid aptamer molecule is preferably targeted to a highly conserved region of the HIV envelope glycoprotein such that specific treatment of a disease or condition can be provided with either one or several nucleic acid molecules of the invention. Such nucleic acid molecules can be delivered exogenously to specific tissue or cellular targets as required. Alternatively, the nucleic acid molecules can be expressed from DNA and/or RNA vectors that are delivered to specific cells.

As used herein “cell” is used in its usual biological sense, and does not refer to an entire multicellular organism, e.g., specifically does not refer to a human. The cell can be present in an organism, e.g., birds, plants and mammals such as humans, cows, sheep, apes, monkeys, swine, dogs, and cats. The cell can be prokaryotic (e.g., bacterial cell) or eukaryotic (e.g., mammalian or plant cell).

By “HIV envelope glycoprotein” is meant, a protein or a mutant protein derivative thereof, comprising sequence expressed and/or encoded by the HIV env gene. Non-limiting examples of the HIV envelope glycoprotein are represented by Genbank Accession Nos. AAM09869-AAM09880. HIV envelope glycoproteins contemplated by the invention include gp120 and gp41.

By “highly conserved nucleic acid binding region” is meant an amino acid sequence of one or more regions in a target protein that does not vary significantly from one generation to the other or from one biological system to the other.

The enzymatic nucleic acid-based modulators of HIV fusogenic activity are useful for the prevention of the diseases and conditions including HIV infection, AIDS, and any other diseases or conditions that are related to the levels of HIV in a cell or tissue.

By “related to the levels of HIV” is meant that the reduction of HIV fusogenic activity and cell entry and/or gene expression (specifically HIV gene) and thus reduction in the level of the HIV expression in an organism will relieve, to some extent, the symptoms of the disease or condition.

The nucleic acid-based modulators of the invention are added directly, or can be complexed with cationic lipids, packaged within liposomes, or otherwise delivered to target cells or tissues. The nucleic acid or nucleic acid complexes can be locally administered to relevant tissues ex vivo, or in vivo through injection, infusion pump or stent, with or without their incorporation in biopolymers. In particular embodiments, the nucleic acid molecules of the invention comprise sequences shown in Tables III-XI. Examples of such nucleic acid molecules consist essentially of sequences defined in the tables.

In another aspect, the invention provides mammalian cells containing one or more nucleic acid molecules and/or expression vectors of this invention. The one or more nucleic acid molecules can independently be targeted to the same or different sites.

In another aspect of the invention, nucleic acid molecules of the invention are expressed from transcription units inserted into DNA or RNA vectors. The recombinant vectors are preferably DNA plasmids or viral vectors. Nucleic acid expressing viral vectors can be constructed based on, but not limited to, adeno-associated virus, retrovirus, adenovirus, or alphavirus. Preferably, the recombinant vectors capable of expressing nucleic acid molecules of the invention are delivered as described above, and persist in target cells. Alternatively, viral vectors may be used that provide for transient expression of the nucleic acid molecules of the invention. Such vectors might be repeatedly administered as necessary. Once expressed, the nucleic acid molecules of the invention bind to the target protein, RNA and/or DNA and modulate its function or expression. Delivery of nucleic acid expressing vectors can be systemic, such as by intravenous or intramuscular administration, by administration to target cells ex-planted from the patient followed by reintroduction into the patient, or by any other means that would allow for introduction into the desired target cell. DNA based nucleic acid molecules of the invention can be expressed via the use of a single stranded DNA intracellular expression vector.

By RNA is meant a molecule comprising at least one ribonucleotide residue. By “ribonucleotide” is meant a nucleotide with a hydroxyl group at the 2′ position of a β-D-ribo-furanose moiety.

By “vectors” is meant any nucleic acid- and/or viral-based technique used to express and/or deliver a desired nucleic acid.

By “patient” or “subject” is meant an organism, which is a donor or recipient of explanted cells or the cells themselves. “Patient” also refers to an organism to which the nucleic acid molecules of the invention can be administered. In one embodiment, a patient is a mammal or mammalian cells. In another embodiment, a patient is a human or human cells.

The nucleic acid molecules of the instant invention, individually, or in combination or in conjunction with other drugs, can be used to treat diseases or conditions discussed herein. For example, to treat a disease or condition associated with the levels of HIV, the nucleic acid molecules can be administered to a patient or can be administered to other appropriate cells evident to those skilled in the art, individually or in combination with one or more drugs under conditions suitable for the treatment.

In a further embodiment, the described molecules, such as aptamers, siRNA, antisense, or enzymatic nucleic acids, can be used in combination with other known treatments to treat conditions or diseases discussed above. For example, the described molecules could be used in combination with one or more known therapeutic agents to treat HIV infection and/or AIDS. Such therapeutic agents may include, but are not limited to, reverse transcriptase inhibitors such as zidovudine (AZT), zalcitabine (DDC), zidovudine (ZDV), lamivudine (3TC), didanosinedelavirdine (DDI), stavudine (D4T), abacavir, efavirenz, nevirapine, or tenofovir disoproxil fumarate, ribavirin and/or protease inhibitors such as indinavir, amprenavir, saquinavir, lopinavir, ritonavir, or nelfinavir, or any combination thereof under conditions suitable for said treatment.

Other features and advantages of the invention will be apparent from the following description of the preferred embodiments thereof, and from the claims.

### BRIEF DESCRIPTION OF THE DRAWINGS

FIG. 1 is a schematic design which outlines the steps involved in HIV cell fusion and entry.

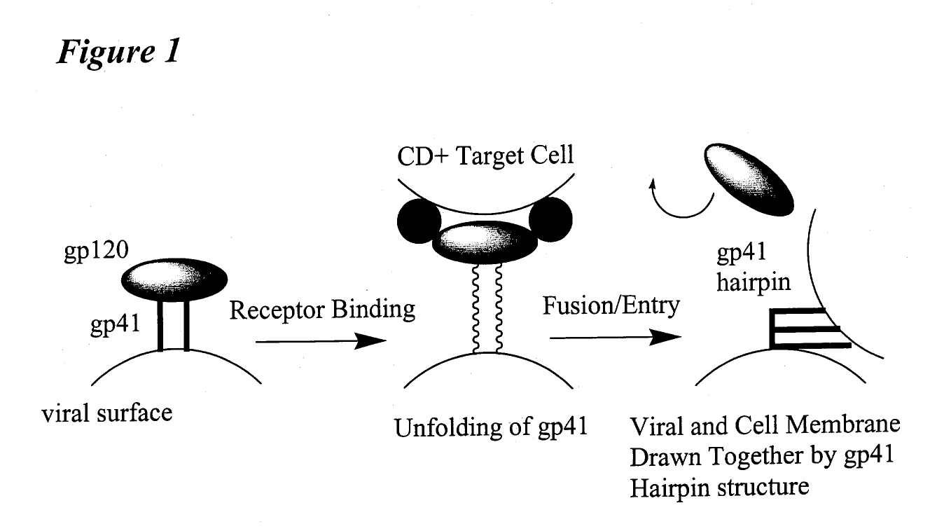

FIG. 2 is a schematic design that shows a non-limiting example of inhibition of HIV cell fusion and entry.

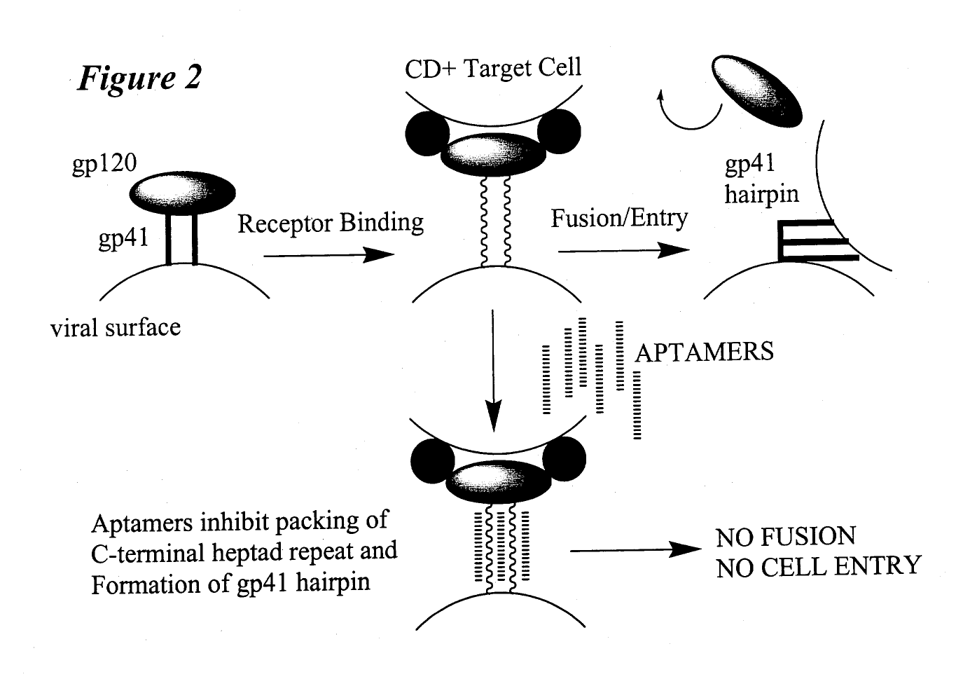

### DETAILED DESCRIPTION OF THE INVENTION

Mechanism of Action of Nucleic Acid Molecules of the Invention

Aptamer: Nucleic acid aptamers can be selected to specifically bind to a particular ligand of interest (see for example Gold et al., U.S. Pat. Nos. 5,567,588 and 5,475,096, Gold et al., 1995, Annu. Rev. Biochem., 64, 763; Brody and Gold, 2000, J. Biotechnol., 74, 5; Sun, 2000, Curr. Opin. Mol. Ther., 2, 100; Kusser, 2000, J. Biotechnol., 74, 27; Hermann and Patel, 2000, Science, 287, 820; and Jayasena, 1999, Clinical Chemistry, 45, 1628). For example, the use of in vitro selection can be applied to evolve nucleic acid aptamers with binding specificity for HIV envelope glycoprotein gp41, gp120 or to any other portion of HIV that disrupts fusogenic activity of the virus. Nucleic acid aptamers can include chemical modifications and linkers as described herein. Nucleic aptamers of the invention can be double stranded or single stranded and can comprise one distinct nucleic acid sequence or more than one nucleic acid sequences complexed with one another. Aptamer molecules of the invention that bind to HIV envelope glycoprotein, for example gp41, can modulate the fusogenic activity of HIV and therefore modulate cell entry and infectivity of the virus.

Antisense: Antisense molecules can be modified or unmodified RNA, DNA, or mixed polymer oligonucleotides and primarily function by specifically binding to matching sequences resulting in modulation of peptide synthesis (Wu-Pong, Nov 1994, BioPharm, 20-33). The antisense oligonucleotide binds to target RNA by Watson Crick base-pairing and blocks gene expression by preventing ribosomal translation of the bound sequences either by steric blocking or by activating RNase H enzyme. Antisense molecules may also alter protein synthesis by interfering with RNA processing or transport from the nucleus into the cytoplasm (Mukhopadhyay & Roth, 1996, Crit. Rev. in Oncogenesis 7, 151-190).

In addition, binding of single stranded DNA to RNA may result in nuclease degradation of the heteroduplex (Wu-Pong, supra; Crooke, supra). To date, the only backbone modified DNA chemistry which will act as substrates for RNase H are phosphorothioates, phosphorodithioates, and borontrifluoridates. Recently, it has been reported that 2′-arabino and 2′-fluoro arabino-containing oligos can also activate RNase H activity.

A number of antisense molecules have been described that utilize novel configurations of chemically modified nucleotides, secondary structure, and/or RNase H substrate domains (Woolf et al., U.S. Pat. No. 5,989,912; Thompson et al., U.S. Serial No. 60/082,404 which was filed on Apr. 20, 1998; Hartmann et al., U.S. Serial No. 60/101,174 which was filed on Sep. 21, 1998) all of these are incorporated by reference herein in their entirety.

Antisense DNA can be used to target RNA by means of DNA-RNA interactions, thereby activating RNase H, which digests the target RNA in the duplex. Antisense DNA can be chemically synthesized or can be expressed via the use of a single stranded DNA intracellular expression vector or the equivalent thereof.

Triplex Forming Oligonucleotides (TFO): Single stranded oligonucleotide can be designed to bind to genomic DNA in a sequence specific manner. TFOs can be comprised of pyrimidine-rich oligonucleotides which bind DNA helices through Hoogsteen Base-pairing (Wu-Pong, supra). In addition, TFOs can be chemically modified to increase binding affinity to target DNA sequences. The resulting triple helix composed of the DNA sense, DNA antisense, and TFO disrupts RNA synthesis by RNA polymerase. The TFO mechanism can result in gene expression or cell death since binding may be irreversible (Mukhopadhyay & Roth, supra) 2′-5′ Oligoadenylates: The 2-5A system is an interferon-mediated mechanism for RNA degradation found in higher vertebrates (Mitra et al., 1996, Proc Nat Acad Sci USA 93, 6780-6785). Two types of enzymes, 2-5A synthetase and RNase L, are required for RNA cleavage. The 2-5A synthetases require double stranded RNA to form 2′-5∝oligoadenylates (2-5A). 2-5A then acts as an allosteric effector for utilizing RNase L, which has the ability to cleave single stranded RNA. The ability to form 2-5A structures with double stranded RNA makes this system particularly useful for modulation of viral replication.

(2′-5′) oligoadenylate structures can be covalently linked to antisense molecules to form chimeric oligonucleotides capable of RNA cleavage (Torrence, supra). These molecules putatively bind and activate a 2-5A-dependent RNase, the oligonucleotide/enzyme complex then binds to a target RNA molecule which can then be cleaved by the RNase enzyme. The covalent attachment of 2′-5′ oligoadenylate structures is not limited to antisense applications, and can be further elaborated to include attachment to nucleic acid molecules of the instant invention.

Enzymatic Nucleic Acid: Several varieties of naturally occurring enzymatic RNAs are presently known (Doherty and Doudna, 2001, Annu. Rev. Biophys. Biomol. Struct., 30, 457-475; Symons, 1994, Curr. Opin. Struct. Biol., 4, 322-30). In addition, several in vitro selection (evolution) strategies (Orgel, 1979, Proc. R. Soc. London, B 205, 435) have been used to evolve new nucleic acid catalysts capable of catalyzing cleavage and ligation of phosphodiester linkages (Joyce, 1989, Gene, 82, 83-87; Beaudry et al., 1992, Science 257, 635-641; Joyce, 1992, Scientific American 267, 90-97; Breaker et al., 1994, TIBTECH 12, 268; Bartel et al., 1993, Science 261:1411-1418; Szostak, 1993, TIBS 17, 89-93; Kumar et al., 1995, FASEB J., 9, 1183; Breaker, 1996, Curr. Op. Biotech., 7, 442; Santoro et al., 1997, Proc. Natl. Acad. Sci., 94, 4262; Tang et al., 1997, RNA 3, 914; Nakamaye & Eckstein, 1994, supra; Long & Uhlenbeck, 1994, supra; Ishizaka et al., 1995, supra; Vaish et al., 1997, Biochemistry 36, 6495). Each can catalyze a series of reactions including the hydrolysis of phosphodiester bonds in trans (and thus can cleave other RNA molecules) under physiological conditions.

Nucleic acid molecules of this invention can block HIV protein expression, specifically, HIV env protein expression, and can be used to treat disease or diagnose disease associated with the levels of HIV.

The enzymatic nature of an enzymatic nucleic acid has significant advantages, such as the concentration of nucleic acid necessary to affect a therapeutic treatment is low. This advantage reflects the ability of the enzymatic nucleic acid molecule to act enzymatically. Thus, a single enzymatic nucleic acid molecule is able to cleave many molecules of target RNA. In addition, the enzymatic nucleic acid molecule is a highly specific modulator, with the specificity of modulation depending not only on the base-pairing mechanism of binding to the target RNA, but also on the mechanism of target RNA cleavage. Single mismatches, or base-substitutions, near the site of cleavage can be chosen to completely eliminate catalytic activity of an enzymatic nucleic acid molecule.

Nucleic acid molecules having an endonuclease enzymatic activity are able to repeatedly cleave other separate RNA molecules in a nucleotide base sequence-specific manner. With proper design and construction, such enzymatic nucleic acid molecules can be targeted to any RNA transcript, and efficient cleavage achieved in vitro (Zaug et al., 324, Nature 429 1986; Uhlenbeck, 1987 Nature 328, 596; Kim et al., 84 Proc. Natl. Acad. Sci. USA 8788, 1987; Dreyfus, 1988, Einstein Quart. J Bio. Med., 6, 92; Haseloff and Gerlach, 334 Nature 585, 1988; Cech, 260 JAMA 3030, 1988; and Jefferies et al., 17 Nucleic Acids Research 1371, 1989; Chartrand et al., 1995, Nucleic Acids Research 23, 4092; Santoro et al., 1997, PNAS 94, 4262).

Because of their sequence specificity, trans-cleaving enzymatic nucleic acid molecules show promise as therapeutic agents for human disease (Usman & McSwiggen, 1995 Ann. Rep. Med. Chem. 30, 285-294; Christoffersen and Marr, 1995 J. Med. Chem. 38, 2023-2037). Enzymatic nucleic acid molecule can be designed to cleave specific RNA targets within the background of cellular RNA. Such a cleavage event renders the RNA non-functional and abrogates protein expression from that RNA. In this manner, synthesis of a protein associated with a disease state can be selectively modulated (Warashina et al., 1999, Chemistry and Biology, 6, 237-250.

The present invention also features nucleic acid sensor molecules or allozymes having sensor domains comprising nucleic acid decoys and/or aptamers of the invention. Interaction of the nucleic acid sensor molecule's sensor domain with a molecular target, such as HIV gp41 or any other suitable HIV target, can activate or inactivate the enzymatic nucleic acid domain of the nucleic acid sensor molecule, such that the activity of the nucleic acid sensor molecule is modulated in the presence of the target-signaling molecule. The nucleic acid sensor molecule can be designed to be active in the presence of the target molecule or alternately, can be designed to be inactive in the presence of the molecular target. For example, a nucleic acid sensor molecule is designed with a sensor domain comprising an aptamer with binding specificity for HIV gp41. In a non-limiting example, interaction of the HIV gp41 with the sensor domain of the nucleic acid sensor molecule can activate the enzymatic nucleic acid domain of the nucleic acid sensor molecule such that the sensor molecule catalyzes a reaction, for example cleavage of HIV RNA. In this example, the nucleic acid sensor molecule is activated in the presence of HIV gp41, and can be used as a therapeutic to treat HIV infection. Alternately, the reaction can comprise cleavage or ligation of a labeled nucleic acid reporter molecule, providing a useful diagnostic reagent to detect the presence of HIV in a system.

Synthesis of Nucleic Acid Molecules

Synthesis of nucleic acids greater than 100 nucleotides in length is difficult using automated methods, and the therapeutic cost of such molecules is prohibitive. In this invention, small nucleic acid motifs (“small” refers to nucleic acid motifs no more than about 100 nucleotides in length, preferably no more than 80 nucleotides in length, and most preferably no more than 50 nucleotides in length; e.g., decoy nucleic acid molecules, aptamer nucleic acid molecules antisense nucleic acid molecules, enzymatic nucleic acid molecules) are preferably used for exogenous delivery. The simple structure of these molecules increases the ability of the nucleic acid to invade targeted regions of protein and/or RNA structure. Exemplary molecules of the instant invention are chemically synthesized, and others can similarly be synthesized.

Oligonucleotides (e.g., DNA oligonucleotides) are synthesized using protocols known in the art, for example as described in Caruthers et al., 1992, Methods in Enzymology 211, 3-19, Thompson et al., International PCT Publication No. WO 99/54459, Wincott et al., 1995, Nucleic Acids Res. 23, 2677-2684, Wincott et al., 1997, Methods Mol. Bio., 74, 59, Brennan et al., 1998, Biotechnol Bioeng., 61, 33-45, and Brennan, U.S. Pat. No. 6,001,311. All of these references are incorporated herein by reference. The synthesis of oligonucleotides makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 2.5 min coupling step for 2′-O-methylated nucleotides and a 45 sec coupling step for 2′-deoxy nucleotides. Table II outlines the amounts and the contact times of the reagents used in the synthesis cycle. Alternatively, syntheses at the 0.2 μmol scale can be performed on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 105-fold excess of S-ethyl tetrazole (60 μL of 0.25 M=15 μmol) can be used in each coupling cycle of 2′-O-methyl residues relative to polymer-bound 5′-hydroxyl. A 22-fold excess (40 μL of 0.11 M=4.4 μmol) of deoxy phosphoramidite and a 70-fold excess of S-ethyl tetrazole (40 μL of 0.25 M=10 μmol) can be used in each coupling cycle of deoxy residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include the following: detritylation solution is 3% TCA in methylene chloride (ABI); capping is performed with 16% N-methyl imidazole in THF (ABI) and 10% acetic anhydride/10% 2,6-lutidine in THF (ABI); and oxidation solution is 16.9 mM 12, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide, 0.05 M in acetonitrile) is used.

Deprotection of the DNA-based oligonucleotides is performed as follows: the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H2O/3:1:1, vortexed and the supernatant is then added to the first supernatant. The combined supernatants, containing the oligoribonucleotide, are dried to a white powder.

The method of synthesis used for normal RNA including certain decoy nucleic acid molecules and enzymatic nucleic acid molecules follows the procedure as described in Usman et al., 1987, J. Am. Chem. Soc., 109, 7845; Scaringe et al., 1990, Nucleic Acids Res., 18, 5433; and Wincott et al., 1995, Nucleic Acids Res. 23, 2677-2684 Wincott et al., 1997, Methods Mol. Bio., 74, 59, and makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 7.5 min coupling step for alkylsilyl protected nucleotides and a 2.5 min coupling step for 2′-O-methylated nucleotides. Table II outlines the amounts and the contact times of the reagents used in the synthesis cycle. Alternatively, syntheses at the 0.2 μmol scale can be done on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 75-fold excess of S-ethyl tetrazole (60 μL of 0.25 M=15 μmol) can be used in each coupling cycle of 2′-O-methyl residues relative to polymer-bound 5′-hydroxyl. A 66-fold excess (120 μL of 0.11 M=13.2 μmol) of alkylsilyl (ribo) protected phosphoramidite and a 150-fold excess of S-ethyl tetrazole (120 μL of 0.25 M=30 μmol) can be used in each coupling cycle of ribo residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include the following: detritylation solution is 3% TCA in methylene chloride (ABI); capping is performed with 16% N-methyl imidazole in THF (ABI) and 10% acetic anhydride/10% 2,6-lutidine in THF (ABI); oxidation solution is 16.9 mM 12, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H2O/3:1:1, vortexed and the supernatant is then added to the first supernatant. The combined supernatants, containing the oligoribonucleotide, are dried to a white powder. The base deprotected oligoribonucleotide is resuspended in anhydrous TEA/HF/NMP solution (300 μL of a solution of 1.5 mL N-methylpyrrolidinone, 750 μL TEA and 1 mL TEA-3HF to provide a 1.4 M HF concentration) and heated to 65° C. After 1.5 h, the oligomer is quenched with 1.5 M NH₄HCO₃.

Alternatively, for the one-pot protocol, the polymer-bound trityl-on oligoribonucleotide is transferred to a 4 mL glass screw top vial and suspended in a solution of 33% ethanolic methylamine/DMSO: 1/1 (0.8 mL) at 65° C. for 15 min. The vial is brought to r.t. TEA-3HF (0.1 mL) is added and the vial is heated at 65° C. for 15 min. The sample is cooled at −20° C. and then quenched with 1.5 M NH₄HCO₃.

For purification of the trityl-on oligomers, the quenched NH₄HCO₃ solution is loaded onto a C-18 containing cartridge that had been prewashed with acetonitrile followed by 50 mM TEAA. After washing the loaded cartridge with water, the RNA is detritylated with 0.5% TFA for 13 min. The cartridge is then washed again with water, salt exchanged with 1 M NaCl and washed with water again. The oligonucleotide is then eluted with 30% acetonitrile.

Inactive hammerhead ribozymes or binding attenuated control (BAC) oligonucleotides are synthesized by substituting a U for Gs and a U for A14 (numbering from Hertel, K. J., et al., 1992, Nucleic Acids Res., 20, 3252). Similarly, one or more nucleotide substitutions can be introduced in other nucleic acid molecules, such as aptamers, to inactivate the molecule and such molecules can serve as a negative control.

The average stepwise coupling yields are typically >98% (Wincott et al., 1995 Nucleic Acids Res. 23, 2677-2684). Those of ordinary skill in the art will recognize that the scale of synthesis can be adapted to be larger or smaller than the example described above including but not limited to 96-well format, all that is important is the ratio of chemicals used in the reaction.

The average stepwise coupling yields are typically >98% (Wincott et al., 1995 Nucleic Acids Res. 23, 2677-2684). Those of ordinary skill in the art will recognize that the scale of synthesis can be adapted to be larger or smaller than the example described above including but not limited to 96-well format, all that is important is the ratio of chemicals used in the reaction.

Alternatively, the nucleic acid molecules of the present invention can be synthesized separately and joined together post-synthetically, for example, by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

The nucleic acid molecules of the present invention can be modified extensively to enhance stability by modification with nuclease resistant groups, for example, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163). Nucleic acid molecules of the invention can be purified by gel electrophoresis using general methods or can be purified by high pressure liquid chromatography (HPLC; see Wincott et al., supra, the totality of which is hereby incorporated herein by reference) and re-suspended in water.

Optimizing Activity of the Nucleic Acid Molecule of the Invention

Chemically synthesizing nucleic acid molecules with modifications (base, sugar and/or phosphate) can prevent their degradation by serum ribonucleases, which can increase their potency (see e.g., Eckstein et al., International Publication No. WO 92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; Gold et al., U.S. Pat. No. 6,300,074; and Burgin et al., supra; all of which are incorporated by reference herein). All of the above references describe various chemical modifications that can be made to the base, phosphate and/or sugar moieties of the nucleic acid molecules described herein. Modifications that enhance their efficacy in cells, and removal of bases from nucleic acid molecules to shorten oligonucleotide synthesis times and reduce chemical requirements are desired.

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-flouro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modification of nucleic acid molecules have been extensively described in the art (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci., 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. Serial No. 60/082,404 which was filed on Apr. 20, 1998; Karpeisky et al., 1998, Tetrahedron Lett., 39, 1131; Earnshaw and Gait, 1998, Biopolymers (Nucleic Acid Sciences), 48, 39-55; Verma and Eckstein, 1998, Annu. Rev. Biochem., 67, 99-134; and Burlina et al., 1997, Bioorg. Med. Chem., 5, 1999-2010; all of the references are hereby incorporated in their totality by reference herein). Such publications describe general methods and strategies to determine the location of incorporation of sugar, base and/or phosphate modifications and the like into ribozymes without modulating catalysis, and are incorporated by reference herein. In view of such teachings, similar modifications can be used as described herein to modify the nucleic acid molecules of the instant invention.

While chemical modification of oligonucleotide internucleotide linkages with phosphorothioate, phosphorothioate, and/or 5′-methylphosphonate linkages improves stability, excessive modifications can cause some toxicity. Therefore, when designing nucleic acid molecules, the amount of these internucleotide linkages should be minimized. The reduction in the concentration of these linkages should lower toxicity, resulting in increased efficacy and higher specificity of these molecules. This period of time varies between hours to days depending upon the disease state. Improvements in the chemical synthesis of RNA and DNA (Wincott et al., 1995 Nucleic Acids Res. 23, 2677; Caruthers et al., 1992, Methods in Enzymology 211,3-19 (incorporated by reference herein)) have expanded the ability to modify nucleic acid molecules by introducing nucleotide modifications to enhance their nuclease stability, as described above.

In one embodiment, nucleic acid molecules of the invention include one or more G-clamp nucleotides. A G-clamp nucleotide is a modified cytosine analog wherein the modifications confer the ability to hydrogen bond both Watson-Crick and Hoogsteen faces of a complementary guanine within a duplex, see for example Lin and Matteucci, 1998, J. Am. Chem. Soc., 120, 8531-8532. A single G-clamp analog substation within an oligonucleotide can result in substantially enhanced helical thermal stability and mismatch discrimination when hybridized to complementary oligonucleotides. The inclusion of such nucleotides in nucleic acid molecules of the invention results in both enhanced affinity and specificity to nucleic acid targets. In another embodiment, nucleic acid molecules of the invention include one or more LNA “locked nucleic acid” nucleotides such as a 2′,4′-C mythylene bicyclo nucleotide (see for example Wengel et al., International PCT Publication No. WO 00/66604 and WO 99/14226).

In another embodiment, the invention features conjugates and/or complexes of nucleic acid molecules targeting HIV. Such conjugates and/or complexes can be used to facilitate delivery of molecules into a biological system, such as a cell. The conjugates and complexes provided by the instant invention can impart therapeutic activity by transferring therapeutic compounds across cellular membranes, altering the pharmacokinetics, and/or modulating the localization of nucleic acid molecules of the invention. The present invention encompasses the design and synthesis of novel conjugates and complexes for the delivery of molecules, including, but not limited to, small molecules, lipids, phospholipids, nucleosides, nucleotides, nucleic acids, antibodies, toxins, negatively charged polymers and other polymers, for example proteins, peptides, hormones, carbohydrates, polyethylene glycols, or polyamines, across cellular membranes. In general, the transporters described are designed to be used either individually or as part of a multi-component system, with or without degradable linkers. These compounds are expected to improve delivery and/or localization of nucleic acid molecules of the invention into a number of cell types originating from different tissues, in the presence or absence of serum (see antibodies, toxins, negatively charged polymers and other polymers, for example proteins, peptides, hormones, carbohydrates, polyethylene glycols, or polyamines, across cellular membranes. In general, the transporters described are designed to be used either individually or as part of a multi-component system, with or without degradable linkers. These compounds are expected to improve delivery and/or localization of nucleic acid molecules of the invention into a number of cell types originating from different tissues, in the presence or absence of serum (see Sullenger and Cech, U.S. Pat. No. 5,854,038). Conjugates of the molecules described herein can be attached to biologically active molecules via linkers that are biodegradable, such as biodegradable nucleic acid linker molecules.

The term “biodegradable nucleic acid linker molecule” as used herein, refers to a nucleic acid molecule that is designed as a biodegradable linker to connect one molecule to another molecule, for example, a biologically active molecule. The stability of the biodegradable nucleic acid linker molecule can be modulated by using various combinations of ribonucleotides, deoxyribonucleotides, and chemically modified nucleotides, for example, 2′-O-methyl, 2′-fluoro, 2′-amino, 2′-O-amino, 2′-C-allyl, 2′-O-allyl, and other 2′-modified or base modified nucleotides. The biodegradable nucleic acid linker molecule can be a dimer, trimer, tetramer or longer nucleic acid molecule, for example, an oligonucleotide of about 2, 3, 4, 5, 6, 7, 8, 9, 10, 11, 12, 13, 14, 15, 16, 17, 18, 19, or 20 nucleotides in length, or can comprise a single nucleotide with a phosphorus-based linkage, for example, a phosphoramidate or phosphodiester linkage. The biodegradable nucleic acid linker molecule can also comprise nucleic acid backbone, nucleic acid sugar, or nucleic acid base modifications.

The term “biodegradable” as used herein, refers to degradation in a biological system, for example enzymatic degradation or chemical degradation.

The term “biologically active molecule” as used herein, refers to compounds or molecules that are capable of eliciting or modifying a biological response in a system. Non-limiting examples of biologically active molecules contemplated by the instant invention include therapeutically active molecules such as antibodies, hormones, antivirals, peptides, proteins, chemotherapeutics, small molecules, vitamins, co-factors, nucleosides, nucleotides, oligonucleotides, enzymatic nucleic acids, antisense nucleic acids, triplex forming oligonucleotides, 2,5-A chimeras, siRNA, dsRNA, allozymes, aptamers, decoys and analogs thereof. Biologically active molecules of the invention also include molecules capable of modulating the pharmacokinetics and/or pharmacodynamics of other biologically active molecules, for example, lipids and polymers such as polyamines, polyamides, polyethylene glycol and other polyethers.

The term “phospholipid” as used herein, refers to a hydrophobic molecule comprising at least one phosphorus group. For example, a phospholipid can comprise a phosphorus-containing group and saturated or unsaturated alkyl group, optionally substituted with OH, COOH, oxo, amine, or substituted or unsubstituted aryl groups.

Therapeutic nucleic acid molecules of the invention delivered exogenously optimally are stable within cells such that therapeutic activity is achieved. The nucleic acid molecules can therefore be designed such that they resistant to nucleases and function as effective intracellular therapeutic agents. Improvements in the chemical synthesis of nucleic acid molecules described in the instant invention and in the art have expanded the ability to modify nucleic acid molecules by introducing nucleotide modifications to enhance their nuclease stability as described above.

In yet another embodiment, nucleic acid molecules having chemical modifications that maintain or enhance enzymatic activity and/or nuclease stability are provided. Such nucleic acids are also generally more resistant to nucleases than unmodified nucleic acids. Thus, in vitro and/or in vivo the activity should not be significantly lowered. As exemplified herein, such nucleic acid molecules are useful in vitro and/or in vivo even if activity over all is reduced 10 fold (Burgin et al., 1996, Biochemistry, 35, 14090).

Use of the nucleic acid-based molecules of the invention will lead to better treatment of the disease progression by affording the possibility of combination therapies (e.g., multiple nucleic acid molecules targeted to different genes; nucleic acid molecules coupled with known small molecule modulators; or intermittent treatment with combinations of molecules (including different motifs) and/or other chemical or biological molecules. The treatment of patients with nucleic acid molecules may also include combinations of different types of nucleic acid molecules.

In another aspect the nucleic acid molecules comprise a 5′ and/or a 3′- cap structure.

By “cap structure” is meant chemical modifications, which have been incorporated at either terminus of the oligonucleotide (see, for example, Wincott et al., WO 97/26270, incorporated by reference herein). These terminal modifications protect the nucleic acid molecule from exonuclease degradation, and may help in delivery and/or localization within a cell. The cap may be present at the 5′-terminus (5′-cap) or at the 3′-terminal (3′-cap) or may be present on both termini. In non-limiting examples the 5′-cap is selected from inverted abasic residue (moiety); 4′,5′-methylene nucleotide; 1-(beta-D-erythrofuranosyl) nucleotide, 4′-thio nucleotide; carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details, see Wincott et al., International PCT publication No. WO 97/26270, incorporated by reference herein).

In another embodiment, the 3′-cap is selected from 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate; 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein).

By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine.

An “alkyl” group refers to a saturated aliphatic hydrocarbon, including straight-chain, branched-chain, and cyclic alkyl groups. Preferably, the alkyl group has 1 to 12 carbons. More preferably, it is a lower alkyl of from 1 to 7 carbons, more preferably 1 to 4 carbons. The alkyl group can be substituted or unsubstituted. When substituted the substituted group(s) is preferably, hydroxyl, cyano, alkoxy, ═O, ═S, NO₂ or N(CH₃)₂, amino, or SH. The term also includes alkenyl groups that are unsaturated hydrocarbon groups containing at least one carbon-carbon double bond, including straight-chain, branched-chain, and cyclic groups. Preferably, the alkenyl group has 1 to 12 carbons. More preferably, it is a lower alkenyl of from 1 to 7 carbons, more preferably 1 to 4 carbons. The alkenyl group may be substituted or unsubstituted. When substituted the substituted group(s) is preferably, hydroxyl, cyano, alkoxy, ═O, ═S, NO₂, halogen, N(CH₃)₂, amino, or SH. The term “alkyl” also includes alkynyl groups that have an unsaturated hydrocarbon group containing at least one carbon-carbon triple bond, including straight-chain, branched-chain, and cyclic groups. Preferably, the alkynyl group has 1 to 12 carbons. More preferably, it is a lower alkynyl of from 1 to 7 carbons, more preferably 1 to 4 carbons. The alkynyl group may be substituted or unsubstituted. When substituted the substituted group(s) is preferably, hydroxyl, cyano, alkoxy, ═O, ═S, NO₂ or N(CH₃)₂, amino or SH.

Such alkyl groups may also include aryl, alkylaryl, carbocyclic aryl, heterocyclic aryl, amide and ester groups. An “aryl” group refers to an aromatic group that has at least one ring having a conjugated pi electron system and includes carbocyclic aryl, heterocyclic aryl and biaryl groups, all of which may be optionally substituted. The preferred substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above). Carbocyclic aryl groups are groups wherein the ring atoms on the aromatic ring are all carbon atoms. The carbon atoms are optionally substituted. Heterocyclic aryl groups are groups having from 1 to 3 heteroatoms as ring atoms in the aromatic ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted. An “amide” refers to an —C(O)—NH—R, where R is either alkyl, aryl, alkylaryl or hydrogen. An “ester” refers to an —C(O)—OR′, where R is either alkyl, aryl, alkylaryl or hydrogen.

By “nucleotide” as used herein is as recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see, for example, Usman and McSwiggen, supra; Eckstein et al., International PCT Publication No. WO 92/07065; Usman et al., International PCT Publication No. WO 93/15187; Uhlman & Peyman, supra, all are hereby incorporated by reference herein). There are several examples of modified nucleic acid bases known in the art as summarized by Limbach et al., 1994, Nucleic Acids Res. 22, 2183. Some of the non-limiting examples of base modifications that can be introduced into nucleic acid molecules include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents; such bases may be used at any position, for example, within the catalytic core of a nucleic acid decoy molecule and/or in the substrate-binding regions of the nucleic acid molecule.

In one embodiment, the invention features modified nucleic acids, for example aptamers, siRNA, antisense, and enzymatic nucleic acid moelcules with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications, see Hunziker and Leumann, 1995, Nucleic Acid Analogues: Synthesis and Properties, in Modern Synthetic Methods, VCH, 331-417, and Mesmaeker et al., 1994, Novel Backbone Replacements for Oligonucleotides, in Carbohydrate Modifications in Antisense Research, ACS, 24-39.

By “abasic” is meant sugar moieties lacking a base or having other chemical groups in place of a base at the 1′ position, (for more details, see Usman et al., U.S. Pat. No. 5,891,683 and Matulic-Adamic et al., U.S. Pat. No. 5,998,203).

By “unmodified nucleoside” is meant one of the bases adenine, cytosine, guanine, thymine, uracil joined to the 1′ carbon of β-D-ribo-furanose.

By “modified nucleoside” is meant any nucleotide base which contains a modification in the chemical structure of an unmodified nucleotide base, sugar and/or phosphate.

In connection with 2′-modified nucleotides as described for the present invention, by “amino” is meant 2′-NH₂ or 2′-O-NH₂, which may be modified or unmodified. Such modified groups are described, for example, in Eckstein et al., U.S. Pat. No. 5,672,695 and Matulic-Adamic et al., WO 98/28317.

Various modifications to nucleic acid (e.g., aptamer, siRNA, antisense and enzymatic nucleic acid) structure can be made to enhance the utility of these molecules. Such modifications will enhance shelf-life, half-life in vitro, stability, and ease of introduction of such oligonucleotides to the target site, e.g., to enhance penetration of cellular membranes, and confer the ability to recognize and bind to targeted cells.

Administration of Nucleic Acid Molecules

Methods for the delivery of nucleic acid molecules are described in Akhtar et al., 1992, Trends Cell Bio., 2, 139; Delivery Strategies for Antisense Oligonucleotide Therapeutics, ed. Akhtar, 1995, Maurer et al., 1999, Mol. Membr. Biol., 16, 129-140; Hofland and Huang, 1999, Handb. Exp. Pharmacol., 137, 165-192; and Lee et al., 2000, ACS Symp. Ser., 752, 184-192, all of which are incorporated herein by reference. Sullivan et al., PCT WO 94/02595, further describes the general methods for delivery of enzymatic nucleic acid molecules. These protocols can be utilized for the delivery of virtually any nucleic acid molecule. Nucleic acid molecules can be administered to cells by a variety of methods known to those of skill in the art, including, but not restricted to, encapsulation in liposomes, by iontophoresis, or by incorporation into other vehicles, such as hydrogels, cyclodextrins, biodegradable nanocapsules, and bioadhesive microspheres, or by proteinaceous vectors (O'Hare and Normand, International PCT Publication No. WO 00/53722). Alternatively, the nucleic acid/vehicle combination is locally delivered by direct injection or by use of an infusion pump. Direct injection of the nucleic acid molecules of the invention, whether subcutaneous, intramuscular, or intradermal, can take place using standard needle and syringe methodologies, or by needle-free technologies such as those described in Conry et al., 1999, Clin. Cancer Res., 5, 2330-2337 and Barry et al., International PCT Publication No. WO 99/31262. The molecules of the instant invention can be used as pharmaceutical agents. Pharmaceutical agents prevent, modulate the occurrence, or treat (alleviate a symptom to some extent, preferably all of the symptoms) of a disease state in a patient.

Thus, the invention features a pharmaceutical composition comprising one or more nucleic acid(s) of the invention in an acceptable carrier, such as a stabilizer, buffer, and the like. The negatively charged polynucleotides of the invention can be administered (e.g., RNA, DNA or protein) and introduced into a patient by any standard means, with or without stabilizers, buffers, and the like, to form a pharmaceutical composition. When it is desired to use a liposome delivery mechanism, standard protocols for formation of liposomes can be followed. The compositions of the present invention may also be formulated and used as tablets, capsules or elixirs for oral administration, suppositories for rectal administration, sterile solutions, suspensions for injectable administration, and the other compositions known in the art.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or patient, including for example a human. Suitable forms, in part, depend upon the use or the route of entry, for example oral, transdermal, or by injection. Such forms should not prevent the composition or formulation from reaching a target cell (i.e., a cell to which the negatively charged nucleic acid is desirable for delivery). For example, pharmacological compositions injected into the blood stream should be soluble. Other factors are known in the art, and include considerations such as toxicity and forms that prevent the composition or formulation from exerting its effect.

By “systemic administration” is meant in vivo systemic absorption or accumulation of drugs in the blood stream followed by distribution throughout the entire body. Administration routes which lead to systemic absorption include, without limitation: intravenous, subcutaneous, intraperitoneal, inhalation, oral, intrapulmonary and intramuscular. Each of these administration routes expose the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation that can facilitate the association of drug with the surface of cells, such as, lymphocytes and macrophages is also useful. This approach may provide enhanced delivery of the drug to target cells by taking advantage of the specificity of macrophage and lymphocyte immune recognition of abnormal cells, such as cancer cells.

By “pharmaceutically acceptable formulation” is meant, a composition or formulation that allows for the effective distribution of the nucleic acid molecules of the instant invention in the physical location most suitable for their desired activity. Nonlimiting examples of agents suitable for formulation with the nucleic acid molecules of the instant invention include: P-glycoprotein inhibitors (such as Pluronic P85), which can enhance entry of drugs into the CNS (Jolliet-Riant and Tillement, 1999, Fundam. Clin. Pharmacol., 13, 16-26); biodegradable polymers, such as poly (DL-lactide-coglycolide) microspheres for sustained release delivery after intracerebral implantation (Emerich, D F et al, 1999, Cell Transplant, 8, 47-58) (Alkermes, Inc. Cambridge, Mass.); and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies for the nucleic acid molecules of the instant invention include material described in Boado et al., 1998, J. Pharm. Sci., 87, 1308-1315; Tyler et al., 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058.

The invention also features the use of the composition comprising surface-modified liposomes containing poly (ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392). Long-circulating liposomes are also likely to protect drugs from nuclease degradation to a greater extent compared to cationic liposomes, based on their ability to avoid accumulation in metabolically aggressive MPS tissues such as the liver and spleen.

The present invention also includes compositions prepared for storage or administration, which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents may be provided. These include sodium benzoate, sorbic acid and esters of p-hydroxybenzoic acid. In addition, antioxidants and suspending agents may be used.

A pharmaceutically effective dose is that dose required to prevent, inhibit the occurrence of, or treat (alleviate a symptom to some extent, preferably all of the symptoms) a disease state. The pharmaceutically effective dose depends on the type of disease, the composition used, the route of administration, the type of mammal being treated, the physical characteristics of the specific mammal under consideration, concurrent medication, and other factors that those skilled in the medical arts will recognize. Generally, an amount between 0.1 mg/kg and 100 mg/kg body weight/day of active ingredients is administered dependent upon potency of the negatively charged polymer.

The present invention also includes compositions prepared for storage or administration that include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985), hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors that those skilled in the medical arts will recognize. Generally, an amount between 0.1 mg/kg and 100 mg/kg body weight/day of active ingredients is administered dependent upon potency of the negatively charged polymer.

The nucleic acid molecules of the invention and formulations thereof can be administered orally, topically, parenterally, by inhalation or spray, or rectally in dosage unit formulations containing conventional non-toxic pharmaceutically acceptable carriers, adjuvants and/or vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions containing nucleic acid molecules of the invention can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be, for example, inert diluents; such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid find use in the preparation of injectables.

The nucleic acid molecules of the invention can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.1 mg to about 140 mg per kilogram of body weight per day are useful in the treatment of the above-indicated conditions (about 0.5 mg to about 7 g per patient per day). The amount of active ingredient that can be combined with the carrier materials to produce a single dosage form varies depending upon the host treated and the particular mode of administration. Dosage unit forms generally contain between from about 1 mg to about 500 mg of an active ingredient.

It is understood that the specific dose level for any particular patient depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The nucleic acid molecules of the present invention may also be administered to a patient in combination with other therapeutic compounds to increase the overall therapeutic effect. The use of multiple compounds to treat an indication may increase the beneficial effects while reducing the presence of side effects.

In one embodiment, the invention compositions suitable for administering nucleic acid molecules of the invention to specific cell types, such as hepatocytes. For example, the asialoglycoprotein receptor (ASGPr) (Wu and Wu, 1987, J. Biol. Chem. 262, 4429-4432) is unique to hepatocytes and binds branched galactose-terminal glycoproteins, such as asialoorosomucoid (ASOR). Binding of such glycoproteins or synthetic glycoconjugates to the receptor takes place with an affinity that strongly depends on the degree of branching of the oligosaccharide chain, for example, triatennary structures are bound with greater affinity than biatenarry or monoatennary chains (Baenziger and Fiete, 1980, Cell, 22, 611-620; Connolly et al., 1982, J. Biol. Chem., 257, 939-945). Lee and Lee, 1987, Glycoconjugate J., 4, 317-328, obtained this high specificity through the use of N-acetyl-D-galactosamine as the carbohydrate moiety, which has higher affinity for the receptor, compared to galactose. This “clustering effect” has also been described for the binding and uptake of mannosyl-terminating glycoproteins or glycoconjugates (Ponpipom et al., 1981, J. Med. Chem., 24, 1388-1395). The use of galactose and galactosamine based conjugates to transport exogenous compounds across cell membranes can provide a targeted delivery approach to the treatment of liver disease such as HBV infection or hepatocellular carcinoma. The use of bioconjugates can also provide a reduction in the required dose of therapeutic compounds required for treatment. Furthermore, therapeutic bioavialability, pharmacodynamics, and pharmacokinetic parameters can be modulated through the use of nucleic acid bioconjugates of the invention.

Alternatively, certain of the nucleic acid molecules of the instant invention can be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591-5; Kashani-Sabet et al., 1992, Antisense Res. Dev., 2, 3-15; Dropulic et al., 1992, J. Virol., 66, 1432-41; Weerasinghe et al., 1991, J. Virol., 65, 5531-4; Ojwang et al., 1992, Proc. Natl. Acad. Sci. USA, 89, 10802-6; Chen et al., 1992, Nucleic Acids Res., 20, 4581-9; Sarver et al., 1990 Science, 247, 1222-1225; Thompson et al., 1995, Nucleic Acids Res., 23, 2259; Good et al., 1997, Gene Therapy, 4, 45; all of these references are hereby incorporated in their totalities by reference herein). Those skilled in the art realize that any nucleic acid can be expressed in eukaryotic cells from the appropriate DNA/RNA vector. The activity of such nucleic acids can be augmented by their release from the primary transcript by a ribozyme (Draper et al., PCT WO 93/23569, and Sullivan et al., PCT WO 94/02595; Ohkawa et al., 1992, Nucleic Acids Symp. Ser., 27, 15-6; Taira et al., 1991, Nucleic Acids Res., 19, 5125-30; Ventura et al., 1993, Nucleic Acids Res., 21, 3249-55; Chowrira et al., 1994, J. Biol. Chem., 269, 25856; all of these references are hereby incorporated in their totality by reference herein).

In another aspect of the invention, RNA molecules of the present invention are preferably expressed from transcription units (see, for example, Couture et al., 1996, TIG., 12, 510) inserted into DNA or RNA vectors. The recombinant vectors are preferably DNA plasmids or viral vectors. Ribozyme expressing viral vectors could be constructed based on, but not limited to, adeno-associated virus, retrovirus, adenovirus, or alphavirus. Preferably, the recombinant vectors capable of expressing the nucleic acid molecules are delivered as described above, and persist in target cells. Alternatively, viral vectors may be used that provide for transient expression of nucleic acid molecules. Such vectors might be repeatedly administered as necessary. Once expressed, the nucleic acid molecule binds to the target mRNA. Delivery of nucleic acid molecule expressing vectors could be systemic, such as by intravenous or intra-muscular

In one aspect, the invention features an expression vector comprising a nucleic acid sequence encoding at least one of the nucleic acid molecules of the instant invention is disclosed. The nucleic acid sequence encoding the nucleic acid molecule of the instant invention is operable linked in a manner which allows expression of that nucleic acid molecule.

In another aspect the invention features an expression vector comprising: a) a transcription initiation region (e.g., eukaryotic pol I, II or III initiation region); b) a transcription termination region (e.g., eukaryotic pol I, II or III termination region); c) a nucleic acid sequence encoding at least one of the nucleic acid catalyst of the instant invention; and wherein said sequence is operably linked to said initiation region and said termination region in a manner which allows expression and/or delivery of said nucleic acid molecule. The vector can optionally include an open reading frame (ORF) for a protein operably linked on the 5′ side or the 3′-side of the sequence encoding the nucleic acid catalyst of the invention; and/or an intron (intervening sequences).

Transcription of the nucleic acid molecule sequences are driven from a promoter for eukaryotic RNA polymerase I (pol I), RNA polymerase II (pol II), or RNA polymerase III (pol III). Transcripts from pol II or pol III promoters will be expressed at high levels in all cells; the levels of a given pol II promoter in a given cell type will depend on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. USA, 87, 6743-7; Gao and Huang 1993, Nucleic Acids Res., 21, 2867-72; Lieber et al., 1993, Methods Enzymol., 217, 47-66; Zhou et al., 1990, Mol. Cell. Biol., 10, 4529-37). All of these references are incorporated by reference herein. Several investigators have demonstrated that nucleic acid molecules, such as ribozymes expressed from such promoters can function in mammalian cells (e.g. Kashani-Sabet et al., 1992, Antisense Res. Dev., 2, 3-15; Ojwang et al., 1992, Proc. Natl. Acad. Sci. USA, 89, 10802-6; Chen et al., 1992, Nucleic Acids Res., 20, 4581-9; Yu et al., 1993, Proc. Natl. Acad. Sci. USA, 90, 6340-4; L'Huillier et al., 1992, EMBO J., 11, 4411-8; Lisziewicz et al., 1993, Proc. Natl. Acad. Sci. U.S.A, 90, 8000-4; Thompson et al., 1995, Nucleic Acids Res., 23, 2259; Sullenger & Cech, 1993, Science, 262, 1566). More specifically, transcription units such as the ones derived from genes encoding U6 small nuclear (snRNA), Proc. Natl. Acad. Sci. USA, 90, 6340-4; L'Huillier et al., 1992, EMBO J., 11, 4411-8; Lisziewicz et al., 1993, Proc. Natl. Acad. Sci. U.S.A, 90, 8000-4; Thompson et al., 1995, Nucleic Acids Res., 23, 2259; Sullenger & Cech, 1993, Science, 262, 1566). More specifically, transcription units such as the ones derived from genes encoding U6 small nuclear (snRNA), transfer RNA (tRNA) and adenovirus VA RNA are useful in generating high concentrations of desired RNA molecules such as ribozymes in cells (Thompson et al., supra; Couture and Stinchcomb, 1996, supra; Noonberg et al., 1994, Nucleic Acid Res., 22, 2830; Noonberg et al., U.S. Pat. No. 5,624,803; Good et al., 1997, Gene Ther., 4, 45; Beigelman et al., International PCT Publication No. WO 96/18736; all of these publications are incorporated by reference herein). The above ribozyme transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review see Couture and Stinchcomb, 1996, supra).

In yet another aspect, the invention features an expression vector comprising nucleic acid sequence encoding at least one of the nucleic acid molecules of the invention in a manner that allows expression of that nucleic acid molecule. The expression vector comprises in one embodiment; a) a transcription initiation region; b) a transcription termination region; c) a nucleic acid sequence encoding at least one said nucleic acid molecule; and wherein said sequence is operably linked to said initiation region and said termination region in a manner which allows expression and/or delivery of said nucleic acid molecule. In another embodiment, the expression vector comprises: a) a transcription initiation region; b) a transcription termination region; c) an open reading frame; d) a nucleic acid sequence encoding at least one said nucleic acid molecule, wherein said sequence is operably linked to the 3′-end of said open reading frame and wherein said sequence is operably linked to said initiation region, said open reading frame and said termination region in a manner which allows expression and/or delivery of said nucleic acid molecule. In yet another embodiment, the expression vector comprises: a) a transcription initiation region; b) a transcription termination region; c) an intron; d) a nucleic acid sequence encoding at least one said nucleic acid molecule and wherein said sequence is operably linked to said initiation region, said intron and said termination region in a manner which allows expression and/or delivery of said nucleic acid molecule. In another embodiment, the expression vector comprises: a) a transcription initiation region; b) a transcription termination region; c) an intron; d) an open reading frame; e) a nucleic acid sequence encoding at least one said nucleic acid molecule, wherein said sequence is operably linked to the 3′-end of said open reading frame and wherein said sequence is operably linked to said initiation region, said intron, said open reading frame and said termination region in a manner which allows expression and/or delivery of said nucleic acid molecule.

### EXAMPLES

The following are non-limiting examples showing the selection, isolation, synthesis and activity of nucleic acids of the instant invention.

### Example 1

### Identification of Aptamers that Specifically Bind to HIV gp41

A nucleic acid aptamer that selectively binds HIV gp41 is provided in accordance with the present invention. The binding affinity of the aptamer for HIV gp41 is preferably represented by the dissociation constant of about 20 nanomolar (nM) or less, and more preferably about 10 nM or less. In one embodiment, the Kd of the aptamer and gp41 target is established using a double filter nitrocellulose filter binding assay such as that disclosed by Wong and Lohman, 1993, PNAS USA, 90, 5428-5432.

Generally, the method for isolating aptamers of the invention having specificity for HIV gp41 comprises: (a) preparing a candidate mixture of potential oligonucleotide ligands for gp41 wherein the candidate mixture is complex enough to contain at least one oligonucleotide ligand for gp41 or a peptide derivative thereof (the gp41 target); (b) contacting the candidate mixture with the gp41 target under conditions suitable for at least one oligonucleotide in the candidate mixture to bind to the gp41 target; (c) removing unbound oligonucleotides from the candidate mixture; (d) collecting the oligonucleotide ligands that are bound to the gp41 target to produce a first collected mixture of oligonucleotide ligands; (e) contacting the mixture from (d) with the gp41 target under more stringent binding conditions than in (b), wherein oligonucleotide ligands having increased affinity to the gp41 target relative to the first collected mixture of (d); (f) removing unbound oligonucleotides from (e); and (g) collecting the oligonucleotide ligands that are bound to the gp41 target to produce a second collected mixture of oligonucleotide ligands to thereby identify oligonucleotides having specificity for HIV gp41. The method can comprise additional steps in which the oligonucleotides isolated in the first or second collected mixture are enriched or expanded by any suitable technique, such as amplification or mutagenesis, prior to contacting the first collected oligonucleotide mixture with the target under the higher stringency conditions, after collecting the oligonucleotides that bound to the target under the higher stringency conditions, or both. Optionally, the contacting and expanding or enriching steps are repeated as necessary to produce the desired aptamer. Thus, it is possible that the second collected oligonucleotide mixture can comprise a single aptamer. The conditions used to affect the stringency of binding used in the method can include varying reaction conditions used for binding, for example the composition of a buffer, temperature, time, and concentration of the components used for binding can be optimized for the desired level of stringency.

In Vitro Selection

In a non-limiting example, aptamers having binding specificity for a HIV-1 gp41 target are isolated by applying the method under the following conditions. First, the gp41 target is attached to a solid matrix such as a bead or chip surface by means of a covalent (e.g. amide or morpholino bond) or non-covalent (eg. biotin/streptavidin) linkage. The gp41 target can comprise the entire isolated gp41 subunit of HIV envelope glycoprotein or an isolated peptide sequence derived therefrom, such as a peptide having SEQ ID NOs. 1233 and/or 1234. The isolated peptide sequence can be synthesized or isolated by protein digest.

A random pool of DNA oligomers is synthesized where the 5′ and 3′ proximal ends are fixed sequences used for amplification and the central region consists of randomized positions. Ten picomoles of template are PCR amplified for 8 cycles in the initial round. Copy DNA of the selected pool of RNA from subsequent rounds of amplification are PCR amplified 18 cycles. PCR reactions are carried out in a 50 .mu.l volume containing 200 picomoles of each primer, 2 mM final concentration dNTP's, 5 units of Thermus aquaticus DNA polymerase (Perkin Elmer Cetus) in a PCR buffer (10 mM Tris-Cl pH 8.4, 50 mM KCl, 7.5 mM MgCl.sub.2, 0.05 mg/ml BSA). Primers are annealed at 58 .degree. C. for 20 seconds and extended at 74 .degree. C. for 2 minutes. Denaturation can occur at 93° C. for 30 seconds.

Products from PCR amplification are used for T7 in vitro transcription in a 200 .mu.l reaction volume. T7 transcripts are purified from an 8 percent, 7M Urea polyacrylamide gel and eluted by crushing gel pieces in a Sodium Acetate/EDTA solution. For each round of amplification, 50 picomoles of the selected pool of RNA is phosphatased for 30 minutes using Calf Intestinal Alkaline Phosphatase. The reaction is then phenol extracted 3 times and chloroform extracted once, then ethanol precipitated. 25 picomoles of this RNA is 5′ end-labeled using gamma ³²P ATP with T4 polynucleotide kinase for 30 minutes. Kinased RNA is gel purified and a small quantity (about 150 fmoles; 100,000 cpm) is used along with 250 picomoles of cold RNA to follow the fraction of RNA bound to gp41 and retained on nitrocellulose filters during the separation step of the method. Typically a protein concentration is used that binds one to five percent of the total input RNA. A control (without protein) is used to determine the background which is typically 0.1% of the total input. Selected RNA is eluted from the filter by extracting three times with water saturated phenol containing 2% lauryl sulfate (SDS), 0.3M NaOAc and 5 mM EDTA followed by a chloroform extraction. Twenty five percent of this RNA is then used to synthesize cDNA for PCR amplification.

Selection with Non-Amlifiable Competitor RNA

In a non-limiting example, selections are performed using two buffer conditions where the only difference between the buffers is sodium concentration (250 mM NaCl or 500 mM NaCl). Two different buffer conditions are used to increase stringency (with the higher salt concentration being more stringent) and to determine whether different ligands can be obtained. After 10 rounds of amplification, the binding constant of the selected pool can decrease by about an order of magnitude and can remain constant for the next two additional rounds. Competitor RNA is not used in the first 12 rounds. After this round, the pool is split and selection carried out in the presence and absence (control) of competitor RNA. For rounds 12 through 18, a 50-fold excess of a non-amplifiable random pool of RNA is present during selection to compete with non-specific low-affinity binders that may survive and thus be amplified. The competitor RNA, which had a 30N random region, is made as described above for the amplifiable pool RNA; however, the competitor RNA has different primer annealing sequences. Thus, the competitor RNA does not survive the cDNA synthesis or PCR amplification steps. It would be apparent to one skilled in the art that other primer sequences could be used as long as they are not homologous to those used for the pool RNA. The use of competitor RNA can increase the affinity of the selected pool by several orders of magnitude.

Cloning and Sequencing

In a non-limiting example, PCR amplified DNA from the last round selected-pool of RNA is phenol and chloroform extracted and ethanol precipitated. The extracted PCR DNA is then digested using Bam HI and Hind III restriction enzymes and sub-cloned into pUC18. DNAs are phenol and chloroform extracted following digestion. Ligation is carried out at room temperature for two hours after which time the reaction is phenol and chloroform extracted and used to electroporate competent cells. Fifty transformants from the selections using competitor RNA at both NaCl concentrations are picked and their DNAs sequenced.

Binding Assays

In a non-limiting example, binding assays are performed by adding 5 .mu.l of HIV-1 gp41 protein, at the appropriate concentrations (i.e., ranging from 2×10⁻⁶ with 3 fold dilutions to 9×10⁻⁹ for 250 mM NaCl and 0.5×10⁻⁷ with 3 fold dilutions to 2×10⁻¹⁰ for 50 mM NaCl), to 45 ul of binding buffer (50 mM Na-HEPES pH 7.5, 250 mM NaCl, 2 mM DTT, 10 mM MnCl₂, 5 mM CHAPS) on ice, then adding 50,000 cpm of kinased RNA (<200 fmoles) in a volume of 3 to 4 .mu.l. This mix is incubated at 37° C. for 20 minutes. The reactions are then passed over nitrocellulose filters, which are pre-equilibrated in buffer, and washed with a 50 mM Tris-Cl pH 7.5 solution. Filters are dried and counted.

General Considerations in Aptamer Selection

When a consensus sequence is identified, oligonucleotides that contain that sequence can be made by conventional synthetic or recombinant techniques. These aptamers can also function as target-specific aptamers of this invention. Such an aptamer can conserve the entire nucleotide sequence of an isolated aptamer, or can contain one or more additions, deletions or substitutions in the nucleotide sequence, as long as a consensus sequence is conserved. A mixture of such aptamers can also function as target-specific aptamers, wherein the mixture is a set of aptamers with a portion or portions of their nucleotide sequence being random or varying, and a conserved region that contains the consensus sequence. Additionally, secondary aptamers can be synthesized using one or more of the modified bases, sugars and linkages described herein using conventional techniques and those described herein.

In some embodiments of this invention, aptamers can be sequenced or mutagenized to identify consensus regions or domains that are participating in aptamer binding to target, and/or aptamer structure. This information is used for generating second and subsequent pools of aptamers of partially known or predetermined sequence. Sequencing used alone or in combination with the retention and selection processes of this invention, can be used to generate less diverse oligonucleotide pools from which aptamers can be made. Further selection according to these methods can be carried out to generate aptamers having preferred characteristics for diagnostic or therapeutic applications. That is, domains that facilitate, for example, drug delivery could be engineered into the aptamers selected according to this invention.

Although this invention is directed to making aptamers using screening from pools of non-predetermined sequences of oligonucleotides, it also can be used to make second-generation aptamers from pools of known or partially known sequences of oligonucleotides. A pool is considered diverse even if one or both ends of the oligonucleotides comprising it are not identical from one oligonucleotide pool member to another, or if one or both ends of the oligonucleotides comprising the pool are identical with non-identical intermediate regions from one pool member to another. Toward this objective, knowledge of the structure and organization of the target protein can be useful to distinguish features that are important for biochemical pathway inhibition or biological response generation in the first generation aptamers. Structural features can be considered in generating a second (less random) pool of oligonucleotides for generating second round aptamers:

Those skilled in the art will appreciate that comparisons of the complete or partial amino acid sequences of the purified protein target to identify variable and conserved regions is useful. Comparison of sequences of aptamers made according to this invention provides information about the consensus regions and consensus sequences responsible for binding. It is expected that certain nucleotides will be rigidly specified and certain positions will exclusively require certain bases. Likewise, studying localized regions of a protein to identify secondary structure can be useful. Localized regions of a protein can adopt a number of different conformations including beta strands, alpha helices, turns (induced principally by proline or glycine residues) or random structure. Different regions of a polypeptide interact with each other through hydrophobic and electrostatic interactions and also by formation of salt bridges, disulfide bridges, etc. to form the secondary and tertiary structures. Defined conformations can be formed within the protein organization, including beta sheets, beta barrels, and clusters of alpha helices.

It sometimes is possible to determine the shape of a protein target or portion thereof by crystallography X ray diffraction or by other physical or chemical techniques known to those skilled in the art. Many different computer programs are available for predicting protein secondary and tertiary structure, the most common being those described in Chou and Fasman, 1978, Biochemistry, 13, 222-245, and Gamier et al., 1978, J. Mol. Biol., 120, 97-120. Generally, these and other available programs are based on the physical and chemical properties of individual amino acids (hydrophobicity, size, charge and presence of side chains) and on the amino acids' collective tendency to form identifiable structures in proteins whose secondary structure has been determined. Many programs attempt to weight structural data with their known influences. For example, amino acids such as proline or glycine are often present where polypeptides have share turns. Long stretches of hydrophobic amino acids (as determined by hydropathy plot), usually have a strong affinity for lipids.

Data obtained by the methods described above and by other conventional methods and tools can be correlated with the presence of particular sequences of nucleotides in the first and second generation aptamers to engineer second and third generation aptamers. Further, according to this invention, second generation aptamers can be identified simply by sequentially screening from pools of oligonucleotides having more predetermined sequences than the pools used in earlier rounds of selection.

These methods can be used to design optimal binding sequences for any desired protein target (which can be portions of aptamers or entire aptamers) and/or to engineer into aptamers any number of desired targeted functions or features. Optimal binding sequences are those which exhibit high relative affinity for target, i.e., affinity measured in Kd in at least in the nanomolar range, and, for certain drug applications, the nanomolar or picomolar range. In practicing this invention, studying the binding energies of aptamers using standard methods known generally in the art are useful. Generally, consensus regions can be identified by comparing the conservation of nucleotides for appreciable enhancement in binding.

Structural knowledge can be used to engineer aptamers made according to this invention. For example, stem structures in the aptamer pool can be vital components in some embodiments where increased aptamer rigidity is desired. According to the teachings of the instant invention, a randomly generated pool of oligonucleotides having the stem sequences can be generated. After aptamers are identified which contain the stem (i.e., use the stem in primers), cross-linkers can be introduced into the stem to covalently fix the stem in the aptamer structure. Cross-linkers also can be used to fix an aptamer to a target. Once an aptamer has been identified, it can be used, either by linkage to, or use in combination with, other aptamers identified according to these methods. One or more aptamers can be used in this manner to bind to one or more targets.

Techniques Used in Optimizing Aptamer Binding

In order to produce nucleic acid aptamers desirable for use as a pharmaceutical composition, it is desirable that the nucleic acid aptamer have the following characteristics: (1) the nucleic acid aptamer binds to the target in a manner capable of achieving the desired effect on the target; (2) be as small as possible to obtain the desired effect; (3) be as stable as possible; and (4) be a specific ligand to the chosen target. In most, if not all, situations it is preferred that the nucleic acid ligand has the highest possible affinity to the target. Modifications or derivatizations of the ligand that confer resistance to degradation and clearance in situ during therapy, the capability to cross various tissue or cell membrane barriers, or any other accessory properties that do not significantly interfere with affinity for the target molecule can also be provided as improvements.

One of the uses of nucleic acid ligands derived by in vitro selection or another approach is to find ligands that alter target molecule function. Thus, it is a good procedure to first assay for inhibition or enhancement of function of the target protein. One could even perform such functional tests of the combined ligand pool prior to cloning and sequencing. Assays for the biological function of the chosen target are generally available and known to those skilled in the art, and can be easily performed in the presence of the nucleic acid ligand to determine if inhibition occurs.

Enrichment can supply a number of cloned ligands of probable variable affinity for the target molecule. Sequence comparisons can yield consensus secondary structures and primary sequences that allow grouping of the ligand sequences into motifs. Although a single ligand sequence (with some mutations) can be found frequently in the total population of cloned sequences, the degree of representation of a single ligand sequence in the cloned population of ligand sequences cannot absolutely correlate with affinity for the target molecule. Therefore mere abundance is not the sole criterion for judging “winners” after in vitro selection and binding assays for various ligand sequences (adequately defining each motif that is discovered by sequence analysis) are required to weigh the significance of the consensus arrived at by sequence comparisons. The combination of sequence comparison and affinity assays should guide the selection of candidates for more extensive ligand characterization.

An important step for determining the length of sequence relevant to specific affinity is to establish the boundaries of that information within a ligand sequence. This is conveniently accomplished by selecting end-labeled fragments from hydrolyzed pools of the ligand of interest so that 5′ and 3′ boundaries of the information can be discovered. To determine a 3′ boundary, one can perform a large-scale in vitro transcription of the amplified aptamer sequence, gel purify the RNA using UV shadowing on an intensifying screen, phosphatasing the purified RNA, phenol extracting extensively, labeling by kinase reactions with 32P, and gel purification of the labeled product (for example by using a film of the gel as a guide). The resultant product can then be subjected to pilot partial digestions with RNase T1 (varying enzyme concentration and time, at 50° C. in a buffer of 7M urea, 50 mM sodium citrate pH 5.2) and alkaline hydrolysis (at 50 mM NaCO3, adjusted to pH 9.0 by prior mixing of 1 M bicarbonate and carbonate solutions; test over ranges of 20 to 60 minutes at 95° C.). Once optimal conditions for alkaline hydrolysis are established (so that there is an even distribution of small to larger fragments) one can scale up to provide enough material for selection by the target (for example on nitrocellulose filters). Binding assays can the be set up, which vary target protein concentration from the lowest saturating protein concentration to that protein concentration at which approximately 10% of RNA is bound as determined by the binding assays for the ligand. One can vary target concentration by increasing volume rather than decreasing the absolute amount of target; this provides a good signal to noise ratio as the amount of RNA bound to the filter is limited by the absolute amount of target. The RNA is eluted as, for example, in in vitro selection and then run on a denaturing gel with T1 partial digests so that the positions of hydrolysis bands can be related to the ligand sequence.

The 5′ boundary can be similarly determined. Large-scale in vitro transcriptions are purified as described herein. There are two methods for labeling the 3′ end of the RNA. One method is to kinase Cp with 32P (or purchase 32P-Cp) and ligate to the purified RNA with RNA ligase. The labeled RNA is then purified and subjected to very identical protocols. An alternative is to subject unlabeled RNAs to partial alkaline hydrolyses and extend an annealed, labeled primer with reverse transcriptase as the assay for band positions. One of the advantages over pCp labeling is the ease of the procedure, the more complete sequencing ladder (by dideoxy chain termination sequencing) with which one can correlate the boundary, and increased yield of assayable product. A disadvantage is that the extension on eluted RNA sometimes contains artifactual stops, so it can be important to control by spotting and eluting starting material on nitrocellulose filters without washes and assaying as the input RNA. Using techniques as described herein, it is possible to find the boundaries of the sequence information required for high affinity binding to the target.

Assessment of Nucleotide Contributions to Aptamer Target Binding Affinity

Once a minimal high affinity ligand sequence is identified, the sequence can be used to identify the nucleotides within the boundaries that are critical to the interaction with the target molecule. One method is to create a new random template in which all of the nucleotides of a high affinity ligand sequence are partially randomized or blocks of randomness are interspersed with blocks of complete randomness for use in an in vitro selection method for example, preferably a modified in vitro selection method as disclosed herein. Such “secondary” in vitro selections produce a pool of ligand sequences in which critical nucleotides or structures are absolutely conserved, less critical features preferred, and unimportant positions unbiased. Secondary in vitro selections can thus help to further elaborate a consensus that is based on relatively few ligand sequences. In addition, even higher-affinity ligands can be provided whose sequences were unexplored in the original in vitro selection.

Another method is to test oligo-transcribed variants (i.e. nucleotide substitution) where the consensus sequence can be unclear. An additional useful set of techniques are inclusively described as chemical modification experiments. Such experiments can be used to probe the native structure of RNAs, by comparing modification patterns of denatured and non-denatured states. The chemical modification pattern of an RNA ligand that is subsequently bound by target molecule can be different from the native pattern, indicating potential changes in structure upon binding or protection of groups by the target molecule. In addition, RNA ligands can fail to be bound by the target molecule when modified at positions critical to either the bound structure of the ligand or critical to interaction with the target molecule. Such experiments in which these positions are identified are described as “chemical modification interference” experiments.

There are a variety of available reagents to conduct such experiments that are known to those skilled in the art (see for example, Ehresmann et al., 1987, Nuc. Acids. Res., 15, 9109-9128. Chemicals that modify bases can be used to modify ligand RNAs. A pool is bound to the target at varying concentrations and the bound RNAs recovered (such as in the boundary experiments) and the eluted RNAs analyzed for the modification. An assay can be by subsequent modification-dependent base removal and aniline scission at the baseless position or by reverse transcription assay of sensitive (modified) positions. In such assays, bands (indicating modified bases) in unselected RNAs, appear that disappear relative to other bands in target protein-selected RNAs. Similar chemical modifications with ethyl nitrosourea, or via mixed chemical or enzymatic synthesis with, for example, 2′-methoxys on ribose or phosphorothioates can be used to identify essential atomic groups on the oligonucleotide backbone. In experiments with 2′-methoxy versus 2′-OH mixtures, the presence of an essential OH group can result in enhanced hydrolysis relative to other positions in molecules that have been stringently selected by the target.

Comparisons of the intensity of bands for bound and unbound ligands can reveal not only modifications that interfere with binding, but also modifications that enhance binding. A ligand can be made with precisely that modification and tested for the enhanced affinity. Thus chemical modification experiments can be a method for exploring additional local contacts with the target molecule, just as walking experiments (see below) are for additional nucleotide level contacts with adjacent domains.

A consensus of primary and secondary structures that enables the chemical or enzymatic synthesis of oligonucleotide ligands whose design is based on that consensus is provided herein via an in vitro selection method, preferably a modified in vitro selection method as disclosed herein. Because the replication machinery of in vitro selection requires that rather limited variation at the subunit level (ribonucleotides, for example), such ligands imperfectly fill the available atomic space of a target molecule's binding surface. However, these ligands can be thought of as high-affinity scaffolds that can be derivatized to make additional contacts with the target molecule. In addition, the consensus contains atomic group descriptors that are pertinent to binding and atomic group descriptors that are coincidental to the pertinent atomic group interactions. Such derivatization does not exclude incorporation of cross-linking agents that will give specifically directly covalent linkages to the target protein. Such derivatization analyses can be performed at but are not limited to the 2′ position of the ribose, and thus can also include derivatization at any position in the base or backbone of the nucleotide ligand.

The present invention thus includes nucleic acid ligands wherein certain chemical modifications have been made in order to increase the in vivo stability of the ligand or to enhance or mediate the delivery of the ligand. Examples of such modifications include chemical substitutions at the ribose and/or phosphate positions of a given RNA sequence. A logical extension of this analysis is a situation in which one or a few nucleotides of the polymeric ligand are used as a site for chemical derivative exploration. The rest of the ligand serves to anchor in place this monomer (or monomers) on which a variety of derivatives are tested for non-interference with binding and for enhanced affinity. Such explorations can result in small molecules that mimic the structure of the initial ligand framework, and have significant and specific affinity for the target molecule independent of that nucleic acid framework. Such derivatized subunits, which can have advantages with respect to mass production, therapeutic routes of administration, delivery, clearance or degradation than the initial ligand, can become the therapeutic and can retain very little of the original ligand. Thus, the aptamer ligands of the present invention can allow directed chemical exploration of a defined site on the target molecule known to be important for the target function.

Walking Experiments

After a minimal consensus ligand sequence has been determined for a given target, it is possible to add random sequence to the minimal consensus ligand sequence and evolve additional contacts with the target, perhaps to separate but adjacent domains. This procedure has been referred to in the art as “walking”. A walking experiment can involve two experiments performed sequentially. A new candidate mixture is produced in which each of the members of the candidate mixture has a fixed nucleic acid region that corresponds to a nucleic acid ligand of interest. Each member of the candidate mixture also contains a randomized region of sequences. According to this method it is possible to identify what are referred to as “extended” nucleic acid ligands, which contain regions that can bind to more than one binding domain of a target.

Covariance Analysis

In conjunction with empirical methods for determining the three dimensional structure of nucleic acids, computer modeling methods for determining structure of nucleic acid ligands can also be employed. Secondary structure prediction is a useful guide to correct sequence alignment. It is also a highly useful stepping-stone to correct 3D structure prediction, by constraining a number of bases into A-form helical geometry.

Tables of energy parameters for calculating the stability of secondary structures exist. Although early secondary structure prediction programs attempted to simply maximize the number of base-pairs formed by a sequence, most current programs seek to find structures with minimal free energy as calculated by these thermodynamic parameters. There are two problems in this approach that should be borne in mind. First, the thermodynamic rules are inherently inaccurate, typically to 10% or so, and there are many different possible structures lying within 10% of the global energy minimum. Second, the actual secondary structure need not lie at a global energy minimum, depending on the kinetics of folding and synthesis of the sequence. Nonetheless, for short sequences, these caveats are of minor importance because there are so few possible structures that can form.

The brute force predictive method is a dot-plot: make an N by N plot of the sequence against itself, and mark an X everywhere a base pair is possible. Diagonal runs of X's mark the location of possible helices. Exhaustive tree-searching methods can then search for all possible arrangements of compatible (i.e., non-overlapping) helices of length L or more; energy calculations can be done for these structures to rank them as more or less likely. The advantages of this method are that all possible topologies, including pseudoknotted conformations, can be examined, and that a number of suboptimal structures are automatically generated as well. An elegant predictive method, and currently the most used, is the Zuker program. Zuker, 1989, Science, 244, 48-52. Originally based on an algorithm developed by Ruth Nussinov, the Zuker program makes a major simplifying assumption that no pseudoknotted conformations will be allowed. This permits the use of a dynamic programming approach that runs in time proportional to only N3 to N4, where N is the length of the sequence. The Zuker program is the only program capable of rigorously dealing with sequences of than a few hundred nucleotides, so it has come to be the most commonly used by biologists. However, the inability of the Zuker program to predict pseudoknotted conformations is a serious consideration. Where pseudoknotted RNA structures are suspected or are recognized by eye, a brute-force method capable of predicting pseudoknotted conformations should be employed.

A central element of comparative sequence analysis is sequence covariations. A covariation is when the identity of one position depends on the identity of another position; for instance, a required Watson-Crick base pair shows strong covariation in that knowledge of one of the two positions gives absolute knowledge of the identity at the other position. Covariation analysis has been used previously to predict the secondary structure of RNAs for which a number of related sequences sharing a common structure exist, such as tRNA, rRNAs, and group I introns. It is now apparent that covariation analysis can be used to detect tertiary contacts as well. Stormo and Gutell, 1992, Nucleic Acids Research, 29, 5785-5795 have designed and implemented an algorithm that precisely measures the amount of covariations between two positions in an aligned sequence set. The program is called “MIXY”-Mutual Information at position X and Y. Consider an aligned sequence set. In each column or position, the frequency of occurrence of A, C, G, U, and gaps is calculated. This frequency is called f(bx), the frequency of base b in column x. Considering two columns at once, the frequency that a given base b appears in column x is f(bx) and the frequency that a given base b appears in column y is f(by). If position x and position y do not care about each other's identity—that is, the positions are independent; there is no covariation—the frequency of observing bases bx and by at position x and y in any given sequence should be just f(bxby)=f(bx)f(by). If there are substantial deviations of the observed frequencies of pairs from their expected frequencies, the positions are said to covary.

The amount of deviation from expectation can be quantified with an information measure M(x,y), the mutual information of x and y.

$$M ( x , y ) = {\sum }_{{b}_{x} {b}_{y}}^{} f ( {b}_{x} {b}_{y} ) In \frac{f ( {b}_{x} {b}_{y} )}{f ( {b}_{x} ) f ( {b}_{y} )}$$

M(x,y) can be described as the number of bits of information one learns about the identity of position y from knowing just the identity of position x. If there is no covariation, M(x,y) is zero; larger values of M(x,y) indicate strong covariation. Covariation values can be used to develop three-dimensional structural predictions.

In some ways, the problem is similar to that of structure determination by NMR. Unlike crystallography, which in the end yields an actual electron density map, NMR yields a set of interatomic distances. Depending on the number of interatomic distances one can get, there can be one, few, or many 3D structures with which they are consistent. Mathematical techniques had to be developed to transform a matrix of interatomic distances into a structure in 3D space. The two main techniques in use are distance geometry and restrained molecular dynamics.

Distance geometry is the more formal and purely mathematical technique. The interatomic distances are considered to be coordinates in an N-dimensional space, where N is the number of atoms. In other words, the “position” of an atom is specified by N distances to all the other atoms, instead of the three (x,y,z) coordinates typically considered. Interatomic distances between every atom are recorded in an N-by-N distance matrix. A complete and precise distance matrix is easily transformed into a 3 by N Cartesian coordinates, using matrix algebra operations. The trick of distance geometry as applied to NMR is dealing with incomplete (only some of the interatomic distances are known) and imprecise data (distances are known to a precision of only a few angstroms at best). Much of the time of distance geometry-based structure calculation is thus spent in pre-processing the distance matrix, calculating bounds for the unknown distance values based on the known ones, and narrowing the bounds on the known ones. Usually, multiple structures are extracted from the distance matrix that are consistent with a set of NMR data; if they all overlap nicely, the data were sufficient to determine a unique structure. Unlike NMR structure determination, covariance gives only imprecise distance values; but also only probabilistic rather than absolute knowledge about whether a given distance constraint should be applied.

Restrained molecular dynamics can also be employed, albeit in a more ad hoc manner.

Given an empirical force field that attempts to describe the forces that all the atoms feel (van der Waals, covalent bonding lengths and angles, electrostatics), one can simulate a number of femtosecond time steps of a molecule's motion, by assigning every atom at a random velocity (from the Boltzmann distribution at a given temperature) and calculating each atom's motion for a femtosecond using Newtonian dynamical equations; that is “molecular dynamics”. In restrained molecular dynamics, one assigns extra ad hoc forces to the atoms when they violate specified distance bounds.

With respect to RNA aptamers, the probabilistic nature of data with restrained molecular dynamics can be addressed. The covariation values can be transformed into artificial restraining forces between certain atoms for certain distance bounds; varying the magnitude of the force according to the magnitude of the covariance. NMR and covariance analysis generates distance restraints between atoms or positions, which are readily transformed into structures through distance geometry or restrained molecular dynamics. Another source of experimental data which can be utilized to determine the three dimensional structures of nucleic acids is chemical and enzymatic protection experiments, which generate solvent accessibility restraints for individual atoms or positions.

### Example 2

### Nucleic Acid Molecules for Modulating HIV env Gene Expression

The following examples demonstrate the selection and design of Enzymatic Nucleic Acid (hammerhead, DNAzyme, NCH, Amberzyme, Zinzyme, or G-Cleaver), Antisense, and siRNA molecules and binding/cleavage sites within HIV RNA.

Identification of Potential Target Sites in Human HIV RNA

The sequence of human HIV genes are screened for accessible sites using a computer-folding algorithm. Regions of the RNA that do not form secondary folding structures and contained potential enzymatic nucleic acid molecule and/or antisense binding/cleavage sites are identified. The sequences of these binding/cleavage sites are shown in Tables III to XI.

### Example 2

### Selection of Enzymatic Nucleic Acid Cleavage Sites in Human HIV RNA

Enzymatic nucleic acid molecule target sites are chosen by analyzing sequences of Human HIV (Genbank accession No: NM_{—}005228) and prioritizing the sites on the basis of folding. Enzymatic nucleic acid molecules are designed that can bind each target and are individually analyzed by computer folding (Christoffersen et al., 1994 J. Mol. Struc. Theochem, 311, 273; Jaeger et al., 1989, Proc. Natl. Acad. Sci. USA, 86, 7706) to assess whether the enzymatic nucleic acid molecule sequences fold into the appropriate secondary structure. Those enzymatic nucleic acid molecules with unfavorable intramolecular interactions between the binding arms and the catalytic core are eliminated from consideration. As noted below, varying binding arm lengths can be chosen to optimize activity. Generally, at least 5 bases on each arm are able to bind to, or otherwise interact with, the target RNA.

### Example 3

### Chemical Synthesis and Purification of Ribozymes and Antisense for Efficient Cleavage and/or Blocking of HIV RNA

Enzymatic nucleic acid molecules and antisense constructs are designed to anneal to various sites in the RNA message. The binding arms of the enzymatic nucleic acid molecules are complementary to the target site sequences described above, while the antisense constructs are fully complementary to the target site sequences described above. The enzymatic nucleic acid molecules and antisense constructs were chemically synthesized. The method of synthesis used followed the procedure for normal RNA synthesis as described above and in Usman et al., (1987 J. Am. Chem. Soc., 109, 7845), Scaringe et al., (1990 Nucleic Acids Res., 18, 5433) and Wincott et al., supra, and made use of common nucleic acid protecting and coupling groups, such as dimethoxytrityl at the 5′-end, and phosphoramidites at the 3′-end. The average stepwise coupling yields were typically >98%.

Enzymatic nucleic acid molecules and antisense constructs are also synthesized from DNA templates using bacteriophage T7 RNA polymerase (Milligan and Uhlenbeck, 1989, Methods Enzymol. 180, 51). Enzymatic nucleic acid molecules and antisense constructs are purified by gel electrophoresis using general methods or are purified by high pressure liquid chromatography (HPLC; See Wincott et al., supra; the totality of which is hereby incorporated herein by reference) and are resuspended in water. The sequences of the chemically enzymatic nucleic acid molecules used in this study are shown below in Tables VI to IX. The sequences of the antisense constructs used in this study are shown in Table X. The sequences of the siRNA constructs used in this study are shown in Table XI.

### Example 4

### Enzymatic Nucleic Acid Molecule Cleavage of HIV RNA Target In Vitro

Enzymatic nucleic acid molecules targeted to the human HIV RNA are designed and synthesized as described above. These enzymatic nucleic acid molecules can be tested for cleavage activity in vitro, for example, using the following procedure. The target sequences and the nucleotide location within the HIV RNA are given in Tables III to IX.

Cleavage Reactions: Full-length or partially full-length, internally-labeled target RNA for enzymatic nucleic acid molecule cleavage assay is prepared by in vitro transcription in the presence of [a-³²P] CTP, passed over a G 50 Sephadex column by spin chromatography and used as substrate RNA without further purification. Alternately, substrates are 5′-³²P-end labeled using T4 polynucleotide kinase enzyme. Assays are performed by pre-warming a 2× concentration of purified enzymatic nucleic acid molecule in enzymatic nucleic acid molecule cleavage buffer (50 mM Tris-HCl, pH 7.5 at 37° C., 10 mM MgCl₂) and the cleavage reaction was initiated by adding the 2× enzymatic nucleic acid molecule mix to an equal volume of substrate RNA (maximum of 1-5 nM) that was also pre-warmed in cleavage buffer. As an initial screen, assays are carried out for 1 hour at 37° C. using a final concentration of either 40 nM or 1 mM enzymatic nucleic acid molecule, i.e., enzymatic nucleic acid molecule excess. The reaction is quenched by the addition of an equal volume of 95% formamide, 20 mM EDTA, 0.05% bromophenol blue and 0.05% xylene cyanol after which the sample is heated to 95° C. for 2 minutes, quick chilled and loaded onto a denaturing polyacrylamide gel. Substrate RNA and the specific RNA cleavage products generated by enzymatic nucleic acid molecule cleavage are visualized on an autoradiograph of the gel. The percentage of cleavage is determined by Phosphor Imager® quantitation of bands representing the intact substrate and the cleavage products.

Indications

Particular degenerative and disease states that can be associated with HIV expression modulation include but are not limited to acquired immunodeficiency disease (AIDS) and related diseases and conditions, including but not limited to Kaposi's sarcoma, lymphoma, cervical cancer, squamous cell carcinoma, cardiac myopathy, rheumatic diseases, and opportunistic infection, for example Pneumocystis carinii, Cytomegalovirus, Herpes simplex, Mycobacteria, Cryptococcus, Toxoplasma, Progressive multifocal leucoencepalopathy (Papovavirus), Mycobacteria, Aspergillus, Cryptococcus, Candida, Cryptosporidium, Isospora belli, Microsporidia and any other diseases or conditions that are related to or will respond to the levels of HIV in a cell or tissue, alone or in combination with other therapies.

The present body of knowledge in HIV research indicates the need for methods to assay HIV activity and for compounds that can regulate HIV expression for research, diagnostic, and therapeutic use.

The use of antiviral compounds, monoclonal antibodies, chemotherapy, radiation therapy, analgesics, and/or anti-inflammatory compounds, are all non-limiting examples of a methods that can be combined with or used in conjunction with the nucleic acid molecules (e.g. aptamers, siRNA, antisense, and enzymatic nucleic acid molecules) of the instant invention. Examples of antiviral compounds that can be used in conjunction with the nucleic acid molecules of the invention include but are not limited to AZT (also known as zidovudine or ZDV), ddC (zalcitabine), ddI (dideoxyinosine), d4T (stavudine), and 3TC (lamivudine) Ribavirin, delvaridine (Rescriptor), nevirapine (Viramune), efravirenz (Sustiva), ritonavir (Norvir), saquinivir (Invirase), indinavir (Crixivan), amprenivir (Agenerase), nelfinavir (Viracept), and/or lopinavir (Kaletra). Common chemotherapies that can be combined with nucleic acid molecules of the instant invention include various combinations of cytotoxic drugs to kill cancer cells. These drugs include but are not limited to paclitaxel (Taxol), docetaxel, cisplatin, methotrexate, cyclophosphamide, doxorubin, fluorouracil carboplatin, edatrexate, gemcitabine, vinorelbine etc. Those skilled in the art will recognize that other drug compounds and therapies can be similarly be readily combined with the nucleic acid molecules of the instant invention are hence within the scope of the instant invention.

Diagnostic Uses

The aptamers of the invention can be used to detect the presence or absence of the target substances to which they specifically bind, such as gp41 or gp120. Such diagnostic tests are conducted by contacting a sample with the aptamer to obtain a complex that is then detected by conventional techniques known in the art. For example, the aptamers can be labeled using radioactive, fluorescent, or chomogenic labels. Interaction of labeled aptamer with the target can result in the detection of the target molecule via an ELISA type assay or sandwich assay, or by other means known in the art. Alternately, the aptamers of the invention can be used to separate or isolate molecules that specifically bind to the aptamer. For example, by coupling the aptamers to a solid support, target molecules which bind to the aptamers can be recovered via affinity chromatography or analyzed by standard means known in the art.

The enzymatic nucleic acid molecules of this invention can be used as diagnostic tools to examine genetic drift and mutations within diseased cells or to detect the presence of HIV RNA in a cell. The close relationship between enzymatic nucleic acid molecule activity and the structure of the target RNA allows the detection of mutations in any region of the molecule which alters the base-pairing and three-dimensional structure of the target RNA. By using multiple enzymatic nucleic acid molecules described in this invention, one can map nucleotide changes which are important to RNA structure and function in vitro, as well as in cells and tissues. Cleavage of target RNAs with enzymatic nucleic acid molecules can be used to inhibit gene expression and define the role (essentially) of specified gene products in the progression of disease. In this manner, other genetic targets can be defined as important mediators of the disease. These experiments can lead to better treatment of the disease progression by affording the possibility of combinational therapies (e.g. multiple enzymatic nucleic acid molecules targeted to different genes, enzymatic nucleic acid molecules coupled with known small molecule inhibitors, or intermittent treatment with combinations of enzymatic nucleic acid molecules and/or other chemical or biological molecules). Other in vitro uses of enzymatic nucleic acid molecules of this invention are well known in the art, and include detection of the presence of mRNAs associated with HIV-related condition. Such RNA is detected by determining the presence of a cleavage product after treatment with an enzymatic nucleic acid molecule using standard methodology.

In a specific example, enzymatic nucleic acid molecules which cleave only wild-type or mutant forms of the target RNA are used for the assay. The first enzymatic nucleic acid molecule is used to identify wild-type RNA present in the sample and the second enzymatic nucleic acid molecule is used to identify mutant RNA in the sample. As reaction controls, synthetic substrates of both wild-type and mutant RNA are cleaved by both enzymatic nucleic acid molecules to demonstrate the relative enzymatic nucleic acid molecule efficiencies in the reactions and the absence of cleavage of the “non-targeted” RNA species. The cleavage products from the synthetic substrates also serve to generate size markers for the analysis of wild-type and mutant RNAs in the sample population. Thus each analysis requires two enzymatic nucleic acid molecules, two substrates and one unknown sample which is combined into six reactions. The presence of cleavage products is determined using an RNAse protection assay so that full-length and cleavage fragments of each RNA can be analyzed in one lane of a polyacrylamide gel. It is not absolutely required to quantify the results to gain insight into the expression of mutant RNAs and putative risk of the desired phenotypic changes in target cells. The expression of mRNA whose protein product is implicated in the development of the phenotype (i.e., HIV) is adequate to establish risk. If probes of comparable specific activity are used for both transcripts, then a qualitative comparison of RNA levels will be adequate and will decrease the cost of the initial diagnosis. Higher mutant form to wild-type ratios are correlated with higher risk whether RNA levels are compared qualitatively or quantitatively. The use of enzymatic nucleic acid molecules in diagnostic applications contemplated by the instant invention is more fully described in George et al., U.S. Pat. Nos. 5,834,186 and 5,741,679, Shih et al., U.S. Pat. No. 5,589,332, Nathan et al., U.S. Pat. No 5,871,914, Nathan and Ellington, International PCT publication No. WO 00/24931, Breaker et al., International PCT Publication Nos. WO 00/26226 and 98/27104, and Sullenger et al., International PCT publication No. WO 99/29842.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. Thus, such additional embodiments are within the scope of the present invention and the following claims.

The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

TABLE I
HIV env sequences (Subtype.Country.isolate year.isolate name)
HIV env sequences
A.BY.97.97BL006
A.GB.-.MA246
A.GB.-.MC108
A.KE.90.K89
A.RW.-.PVPI
A.RW.-.SF1703
A.SE.94.SE7535
A.SE.95.SE8538
A.SE.95.SE8891
A.SE.95.UGSE8131
A.UA.97.ukr970063
A.UG.90.UG273A
A.UG.90.UG275A
A1.KE.93.Q23-17
A1.SE.94.SE7253
A1.UG.85.U455
A1.UG.92.92UG037
A2.CD.-.97CDKS10
A2.CD.-.97CDKTB48
A2.CY.94.94CY017.41
A2C.ZM.89.ZAM174
A2C.ZM.89.ZAM716-3
A2C.ZM.90.ZAM18B
A2D.KR.97.97KR004
A2G.CD.-.97CDKP58
AC.BE.-.VI313
AC.IN.95.95IN21301
AC.RW.92.92RW009
AC.SE.96.SE9488
ACD.SE.95.SE8603
ACG.BE.-.VI1035
AD.KE.90.K124A2
AD.SE.93.SE6954
AD.SE.95.SE7108
AD.UG.-.C6080-10
AD.UG.92.2UG035-22
ADHK.NO.97.97NOGIL3
ADK.CD.85.MAL
AG.BE.-.VI1197
AG.BE.-.VI5251
AG.CD.89.VI191A2
AG.NG.92.92NG003
AGHU.GA.-.VI354
AGJ.BW.98.BW2117
AGU.CD.76.Z321
AU.NG.94.NG3678
AU.NG.95.NG1935
AU.SE.93.SE6594
B.AU.-.VH
B.AU.86.MBC200
B.AU.87.MBC925
B.AU.93.MBC18
B.AU.95.MBCC54
B.AU.96.MBCC98
B.AU.96.MBCD36
B.BE.-.SIMI84
B.CA.-.CA5
B.CN.-.RL42
B.DE.86.D31
B.DE.86.HAN
B.ES.89.89SP061
B.FR.-.PHI120
B.FR.-.PHI133
B.FR.-.PHI146
B.FR.-.PHI153
B.FR.-.PHI159
B.FR.-.PIH155
B.FR.-.PIH160
B.FR.-.PIH309
B.FR.-.PIH373
B.FR.-.PIH374
B.FR.83.HXB2
B.GA.-.OYI,
B.GB.-.AC-46
B.GB.-.CAM1
B.GB.-.GB8.C1
B.GB.-.HIM286332
B.GB.-.HIM286336
B.GB.-.HIM286337
B.GB.-.JB
B.GB.-.M23470
B.GB.-.M24244C3
B.GB.-.M26864
B.GB.-.M30156
B.GB.-.M737677
B.GB.-.M737685
B.GB.-.MB314
B.GB.-.PE052C1
B.GB.-.PE104C38
B.GB.-.PE131C3
B.GB.-.WB
B.GB.59.MANC
B.JP.-.ETR
B.JP.86.JH32
B.KR.-.WK
B.NL.-.68A
B.NL.-.ENVVA
B.NL.-.ENVVF
B.NL.-. ENVVG
B.NL.86.3202A21
B.NL.86.H0320-2A12
B.TH.93.93TH067
B.TT.-.QZ4589
B.TW.-.TWCYS
B.UA.-.UKR1216
B.UNK.-.NL43E9
B.US.-.546BMB4
B.US.-.ADA
B.US.-.BORI
B.US.-BRVA
B.US.-.C26-12.1BH
B.US.-.DH123
B.US.-.M02-3.SW
B.US.-.NC7
B.US.-.P896
B.US.-.SF128A
B.US-.US1
B.US.-.US2
B.US.-.US3
B.US.-.US4
B.US.-.WMJ22
B.US.83.RF
B.US.83.SF2
B.US.84.CDC452
B.US.84.MNCG
B.US.84.NY5CG
B.US.84.SC
B.US.84.SC141
B.US.84.SC14C
B.US.85.85WCIPR54
B.US.85.ALA1
B.US.85.SFMHS11
B.US.85.SFMHS21
B.US.85.SFMHS3
B.US.86.JRCSF
B.US.86.JRFL
B.US.86.SFMHS1
B.US.86.SFMHS16
B.US.86.SFMHS17
B.US.86.SFMHS18
B.US.86.SFMHS2
B.US.86.SFMHS4
B.US.86.SFMHS8
B.US.86.YU2
B.US.87.BC
B.US.87.SFMHS5
B.US.87.SFMHS7
B.US.87.SFMHS9
B.US.88.SFMHS19
B.US.88.SFMHS6
B.US.88.WR27
B.US.89.R2
B.US.89.SFMHS20
B.US.90.WEAU160
B.US.92.92US657.1
BC.CN.-.CHN19
BF1.BR.93.93BR029.4
C.BI.91.BU910112
C.BI.91.8U910213
C.BI.91.BU910316
C.BI.91.BU910423
C.BI.91.BU910518
C.BI.91.BU910611
C.BI.91.BU910717
C.BI.91.BU910812
C.BR.92.92BR025
C.BW.96.96BW01B03
C.BW.96.96BW0402
C.BW.96.96BW0502
C.BW.96.96BW11B01
C.BW.96.96BW1210
C.BW.96.96BW15B03
C.BW.96.96BW16B01
C.BW.96.96BW17B05
C.DJ.91.DJ259A
C.DJ.91.DJ373A
C.ET.86.ETH2220
C.IN.-.HIM276221
C.IN.93.93IN101
C.IN.93.93IN904
C.IN.93.93IN905
C.IN.93.93IN999
C.IN.94.94IN11246
C.IN.95.95IN21068
C.SN.90.SE364A
C.SO.89.SO145A
C.UG.90.UG268A2
CD.BI.91.BU910905
CPZ.CD.-.CPZANT
CPZ.CM.-.CAM3
CPZ.CM.98.CAM5
CPZ.GA.-.CPZGAB
CPZ.US.85.CPZUS
CRF01_AE.CF.90.90CF1
CRF01_AE.CF.90.90CF4
CRF01_AE.CM.-.CA10
CRF01_AE.TH.90.CM240
CRF01_AE.TH.92.TH920
CRF01_AE.TH.92.TH921
CRF01_AE.TH.93.93TH0
CRF01_AE.TH.93.93TH2
CRF01_AE.TH.93.KH03
CRF01_AE.TH.93.KH08
CRF01_AE.TH.94.A0102
CRF01_AE.TH.94.E1142
CRF01_AE.TH.95.95TNI
CRF01_AE.TH.95.N1114
CRF01_AE.TH.95.N1115
CRF01_AE.TH.95.TH022
CRF01_AE.TH.96.N1115
CRF02_AG.CM.97.MP807
CRF02_AG.DJ.91.DJ258
CRF02_AG.FR.91.DJ263
CRF02_AG.FR.91.DJ264
CRF02_AG.GH.-.G829
CRF02_AG.NG.-.IBNG
CRF02_AG.NG.94.NG367
CRF02_AG.NG.95.NG192
CRF02_AG.SE.94.SE781
CRF02_AG.SN.98.MP121
CRF03_AB.RU.-.KAL68.
CRF03_AB.RU.97.KAL15
CRF03_AB.RU.98.RU980
CRF04_cpx.CY.94.94CY
CRF04_cpx.GR.91.97PV
CRF04_cpx.GR.97.97PV
CRF05_DF.BE.-.VI1310
CRF05_DF.BE.93.VI961
CRF06_cpx.AU.96.BFP9
CRF06_cpx.ML.95.95ML
CRF06_cpx.NG.94.NG36
CRF06_cpx.SN.97.97SE
CRF10_CD.TZ.96.96TZB
CRF11_cpx.CM.-.CA1
CRF11_cpx.CM.-.MP818
CRF11_cpx.FR.-.MP129
CRF11_cpx.FR.-.MP130
CRF11_cpx.GR.-.GR17
CRF11_cpx.NG.94.NG36
D.CD.-.JY1
D.CD.83.ELI
D.CD.83.NDK
D.CD.84.84ZR085
D.CD.85.Z2Z6
D.CI.-.CI13
D.SN.90.SE365A2
D.TZ.87.87TZ4622
D.UG.-.C971-412
D.UG.-.WHO15-474
D.UG.90.UG266A2
D.UG.90.UG269A
D.UG.90.UG274A2
D.UG.92.92UG024-D
D.UG.94.94UG1141
F1.BE.93.VI850
F1.BR.89.BZ126
F1.BR.93.93BR020.1
F1.FI.93.FIN9363
F1.FR.96.MP411
F2.CM.-.CA20
F2.CM.-.HIM277819
F2.CM.95.MP255
F2.CM.95.MP257
F2KU.BE.94.VI1126
G.BE.96.DRCBL
G.FI.93.HH8793-12.1
G.GA.-.LBV217
G.NG.92.92NG083
G.NG.95.NG1928
G.NG.95.NG1929
G.NG.95.NG1937
G.NG.95.NG1939
G.SE.93.SE6165
GH.GA.90.VI525
H.BE.93.VI991
H.BE.93.VI997
H.CF.90.90CF056
J.SE.93.SE7887
J.SE.94.SE7022
K.CD.97.EQTB11C
K.CM.96.MP535
MO.CM.97.97CAMP645MO
N.CM.-.YBF106
N.CM.95.YBF30
O.CM.-.ANT70
O.CM.-.CM4974
O.CM.91.MVP5180
O.CM.93.HIV1CA9EN
O.GA.92.VI686
O.GQ.-.193HA
O.GQ.-.276HA
O.GQ.-.341HA
O.SN.99.SEMP1299
O.SN.99.SEMP1300
U.CD.83.83CD003

TABLE II
A. 2.5 μmol Synthesis Cycle ABI 394 Instrument

| | | | | Wait | | | | | |
|---|---|---|---|---|---|---|---|---|---|
| | Equi- | | | Time* | | Wait Time* | | Wait | |
| Reagent | valents | Amount | | DNA | | 2′-O-methyl | | Time*RNA | |
| Phosphor- | 6.5 | 163 | μL | 45 | sec | 2.5 | min | 7.5 | min |
| amidites | | | | | | | | | |
| S-Ethyl | 23.8 | 238 | μL | 45 | sec | 2.5 | min | 7.5 | min |
| Tetrazole | | | | | | | | | |
| Acetic | 100 | 233 | μL | 5 | sec | 5 | sec | 5 | sec |
| Anhydride | | | | | | | | | |
| N-Methyl | 186 | 233 | μL | 5 | sec | 5 | sec | 5 | sec |
| Imidazole | | | | | | | | | |
| TCA | 176 | 2.3 | mL | 21 | sec | 21 | sec | 21 | sec |
| Iodine | 11.2 | 1.7 | mL | 45 | sec | 45 | sec | 45 | sec |
| Beaucage | 12.9 | 645 | μL | 100 | sec | 300 | sec | 300 | sec |

| | | | | | | |
|---|---|---|---|---|---|---|
| Aceto- | NA | 6.67 | mL | NA | NA | NA |
| nitrile | | | | | | |

B. 0.2 μmol Synthesis Cycle ABI 394 Instrument

| | | | | Wait | | | | | |
|---|---|---|---|---|---|---|---|---|---|
| | Equi- | | | Time* | | Wait Time* | | Wait | |
| Reagent | valents | Amount | | DNA | | 2′-O-methyl | | Time*RNA | |
| Phosphor- | 15 | 31 | μL | 45 | sec | 233 | sec | 465 | sec |
| amidites | | | | | | | | | |
| S-Ethyl | 38.7 | 31 | μL | 45 | sec | 233 | min | 465 | sec |
| Tetrazole | | | | | | | | | |
| Acetic | 655 | 124 | μL | 5 | sec | 5 | sec | 5 | sec |
| Anhydride | | | | | | | | | |
| N-Methyl | 1245 | 124 | μL | 5 | sec | 5 | sec | 5 | sec |
| Imidazole | | | | | | | | | |
| TCA | 700 | 732 | μL | 10 | sec | 10 | sec | 10 | sec |
| Iodine | 20.6 | 244 | μL | 15 | sec | 15 | sec | 15 | sec |
| Beaucage | 7.7 | 232 | μL | 100 | sec | 300 | sec | 300 | sec |

| | | | | | | |
|---|---|---|---|---|---|---|
| Aceto- | NA | 2.64 | mL | NA | NA | NA |
| nitrile | | | | | | |

C. 0.2 μmol Synthesis Cycle 96 well Instrument

| | Equivalents: DNA/ | Amount: DNA/2′-O- | | Wait Time* | Wait Time* | Wait Time* |
|---|---|---|---|---|---|---|
| Reagent | 2′-O-methyl/Ribo | methyl/Ribo | | DNA | 2′-O-methyl | Ribo |
| Phosphoramidites | 22/33/66 | 40/60/120 | μL | 60 sec | 180 sec | 360 sec |
| S-Ethyl Tetrazole | 70/105/210 | 40/60/120 | μL | 60 sec | 180 min | 360 sec |
| Acetic Anhydride | 265/265/265 | 50/50/50 | μL | 10 sec | 10 sec | 10 sec |
| N-Methyl | 502/502/502 | 50/50/50 | μL | 10 sec | 10 sec | 10 sec |
| Imidazole | | | | | | |
| TCA | 238/475/475 | 250/500/500 | μL | 15 sec | 15 sec | 15 sec |
| Iodine | 6.8/6.8/6.8 | 80/80/80 | μL | 30 sec | 30 sec | 30 sec |
| Beaucage | 34/51/51 | 80/120/120 | | 100 sec | 200 sec | 200 sec |
| Acetonitrile | NA | 1150/1150/1150 | μL | NA | NA | NA |

*Wait time does not include contact time during delivery.

TABLE III

HIV env target sequences

| | |
|---|---|
| Sequence | Seq ID |

| | |
|---|---|
| AAAAAUAACAUGGUA | 1 |
| AAAAAUAUUCAUAAU | 2 |
| AAAAGAAUGAACAAG | 3 |
| AAAAUAACAUGGUAG | 4 |
| AAACUGCUCUUUCAA | 5 |
| AAAGAAUGAACAAGA | 6 |
| AAAGAGAAGAGUGGU | 7 |
| AAAGCCAUGUGUAAA | 8 |
| AAAGCCUAAAGCCAU | 9 |
| AAAUAACAUGGUAGA | 10 |
| AAAUAUAAAGUAGUA | 11 |
| AACAUGACCUGGAUG | 12 |
| AACAUGUGGAAAAAU | 13 |
| AACGCUGACGGUACA | 14 |
| AACUCACAGUCUGGG | 15 |
| AAGAAGAAGGUGGAG | 16 |
| AAGAGAAGAGUGGUG | 17 |
| AAGCAAUGUAUGCCC | 18 |
| AAGCACUAUGGGCGC | 19 |
| AAGCCUAAAGCCAUG | 20 |
| AAGUGAAUUAUAUAA | 21 |
| AAUAACGCUGACGGU | 22 |
| AAUAGAGUUAGGCAG | 23 |
| AAUAUUCAUAAUGAU | 24 |
| AAUCAGUUUAUGGGA | 25 |
| AAUGAUAGUAGGAGG | 26 |
| AAUGGCAGUCUAGCA | 27 |
| AAUGUACACAUGGAA | 28 |
| AAUGUAUGCCCCUCC | 29 |
| AAUGUCAGCACACUA | 30 |
| AAUUAUAUAAAUAUA | 31 |
| AAUUCCCAUACAUUA | 32 |
| AAUUGGACAAGUGAA | 33 |
| AAUUUGCUGAGGGCU | 34 |
| ACAAUGUACACAUGG | 35 |
| ACAAUUAUUGUCUGG | 36 |
| ACAAUUGGACAAGUG | 37 |
| ACACAUGCCUGUGUA | 38 |
| ACAGACCCCAACCCA | 39 |
| ACAGGCCAGACAAUU | 40 |
| ACAGUACAAUGUACA | 41 |
| ACAGUCUAUUAUGGG | 42 |
| ACAUGCCUGUGUACC | 43 |
| ACAUGUGGAAAAAUA | 44 |
| ACCACCGCUUCAGAG | 45 |
| ACCCACAGACCCCAA | 46 |
| ACCCCAACCCACAAG | 47 |
| ACCUGGAGGAGGAGA | 48 |
| ACCUGUGUGGAAAGA | 49 |
| ACGCUGACGCUACAG | 50 |
| ACGGUACAGGCCAGA | 51 |
| ACUCACAGUCUGGGG | 52 |
| AGAAAAAAGAGCACU | 53 |
| AGAAGAAGGUGGAGA | 54 |
| AGAAGUGAAUUAUAU | 55 |
| AGACAAUUAUUGUCU | 56 |
| AGACCCCAACCCACA | 57 |
| AGACCUGGAGGAGGA | 58 |
| AGAGAAAAAAGAGCA | 59 |
| AGAGAGAAAAAAGAG | 60 |
| AGAGUUAGGCAGGGA | 61 |
| AGAUGCUAAAGCAUA | 62 |
| AGCAAUGUAUGCCCC | 63 |
| AGCACUAUGGGCGCA | 64 |
| AGCAGCAGGAAGCAC | 65 |
| AGCAGGAAGCACUAU | 66 |
| AGCCUAAAGCCAUGU | 67 |
| AGCCUGUGCCUCUUC | 68 |
| AGCUCCAGGCAAGAG | 69 |
| AGGAAGCACUAUGGG | 70 |
| AGGAGGAGAUAUGAG | 71 |
| AGGAGUAGCACCCAC | 72 |
| AGGAUCAACAGCUCC | 73 |
| AGGCAAGAGUCCUGG | 74 |
| AGGCAGGGAUACUCA | 75 |
| AGGGACAAUUGGAGA | 76 |
| AGUACAAUGUACACA | 77 |
| AGUCUAUUAUGGGGU | 78 |
| AGUGAAUUAUAUAAA | 79 |
| AGUUAGGCAGGGAUA | 80 |
| AGUUGGAGUAAUAAA | 81 |
| AGUUUAUGGGAUCAA | 82 |
| AUAAAAAUAUUCAUA | 83 |
| AUAAAUAUAAAGUAG | 84 |
| AUAACGCUGACGGUA | 85 |
| AUAAUCAGUUUAUGG | 86 |
| AUAAUGAUAGUAGGA | 87 |
| AUAGAGUUAGGCAGG | 88 |
| AUAGUGCAACAGCAA | 89 |
| AUAUAAAUAUAAAGU | 90 |
| AUAUAAUCAGUUUAU | 91 |
| AUAUGAGGGACAAUU | 92 |
| AUAUUCAUAAUGAUA | 93 |
| AUCAAAUAUUACAGG | 94 |
| AUCAACAGCUCCUAG | 95 |
| AUCAGAUGCUAAAGC | 96 |
| AUCAGUUUAUGGGAU | 97 |
| AUGAGGGACAAUUGG | 98 |
| AUGAUAGUAGGAGGC | 99 |
| AUGCCUGUGUACCCA | 100 |
| AUGGCAGUCUAGCAG | 101 |
| AUGGGGUACCUGUGU | 102 |
| AUGUACACAUGGAAU | 103 |
| AUGUAUGCCCCUCCC | 104 |
| AUGUCAGCACAGUAC | 105 |
| AUGUGGAAAAAUAAC | 106 |
| AUUAACAAGAGAUGG | 107 |
| AUUAUAUAAAUAUAA | 108 |
| AUUAUGGGGUACCUC | 109 |
| AUUCAUAAUGAUAGU | 110 |
| AUUCCCAUACAUUAU | 111 |
| AUUGGAGAAGUGAAU | 112 |
| AUUGUCUGGUAUAGU | 113 |
| AUUUGCUGAGGGCUA | 114 |
| AUUUUAACAUGUGGA | 115 |
| AUUUUGUGCAUCAGA | 116 |
| CAAAGAGAAGAGUGG | 117 |
| CAAAGCCUAAAGCCA | 118 |
| CAACUCACAGUCUGG | 119 |
| CAAUAACGCUGACGG | 120 |
| CAAUGUACACAUGGA | 121 |
| CAAUGUAUGCCCCUC | 122 |
| CAAUUCCCAUACAUU | 123 |
| CAAUUGGAGAAGUGA | 124 |
| CAAUUUGCUCAGGGC | 125 |
| CACAGACCCCAACCC | 126 |
| CACAGUCUAUUAUGG | 127 |
| CACACUCUGGGGCAU | 128 |
| CACAUCCCUGUGUAC | 129 |
| CACUAUGGGCGCAGC | 130 |
| CAGACAAUUAUUGUC | 131 |
| CAGACCCCAACCCAC | 132 |
| CAGACCUGGAGGAGG | 133 |
| CAGAUGCUAAAGCAU | 134 |
| CAGCACAGUACAAUG | 135 |
| CAGCAGGAAGCACUA | 136 |
| CAGCUCCAGGCAAGA | 137 |
| CAGGAAGCACUAUGG | 138 |
| CAGGCAAGAGUCCUG | 139 |
| CAGGCCAGACAAUUA | 140 |
| CAGUACAAUGUACAC | 141 |
| CAGUCUAUUAUGGGG | 142 |
| CAGUUUAUGGGAUCA | 143 |
| CAUAAUGAUAGUAGG | 144 |
| CAUACAUUAUUGUCC | 145 |
| CAUCAGAUGCUAAAG | 146 |
| CAUGCCUGUGUACCC | 147 |
| CAUGUGGAAAAAUAA | 148 |
| CCAAUUCCCAUACAU | 149 |
| CCACAGACCCCAACC | 150 |
| CCACCGCUUGAGAGA | 151 |
| CCAGACAAUUAUUGU | 152 |
| CCAGGCAAGAGUCCU | 153 |
| CCAUACAUUAUUGUG | 154 |
| CCCAACCCACAAGAA | 155 |
| CCCACAGACCCCAAC | 156 |
| CCCAUACAUUAUUGU | 157 |
| CCCCAACCCACAAGA | 158 |
| CCGCUUGAGAGACUU | 159 |
| CCUAAAGCCAUCUGU | 160 |
| CCUCUUCAGCUACCA | 161 |
| CCUGGAGGAGGAGAU | 162 |
| CCUGGCUGUGGAAAG | 163 |
| CCUGUGCCUCUUCAG | 164 |
| CCUGUGUACCCACAG | 165 |
| CCUUGGGUUCUUGGG | 166 |
| CCUUUGAGCCAAUUC | 167 |
| CGCUGACGGUACAGG | 168 |
| CGGUACAGGCCAGAC | 169 |
| CUAAAGCCAUGUGUA | 170 |
| CUAAAGGAUCAACAG | 171 |
| CUAGUUGGAGUAAUA | 172 |
| CUAUUAUGGGGUACC | 173 |
| CUAUUUUGUUCAUCA | 174 |
| CUCACAGUCUGGGGC | 175 |
| CUCCAGGCAAGAGUC | 176 |
| CUCUGGAAAACUCAU | 177 |
| CUCUUCAGCUACCAC | 178 |
| CUGACGGUACAGGCC | 179 |
| CUGGAGGAGGAGAUA | 180 |
| CUGGCUGUGGAAAGA | 181 |
| CUGGUAUAGUGCAAC | 182 |
| CUGUCCCUCUUCAGC | 183 |
| CUGUGGAAAGAUACC | 184 |
| CUGUGGUAUAUAAAA | 185 |
| CUGUGUACCCACAGA | 186 |
| CUUCAGACCUGGAGG | 187 |
| CUUCAGCUACCACCG | 188 |
| CUUGGGAGCAGCAGG | 189 |
| CUUGGGUUCUUGGGA | 190 |
| CUUUGAGCCAAUUCC | 191 |
| GAAAAAUAACAUGGU | 192 |
| GAAAAGAAUGAACAA | 193 |
| GAAGAAGAAGGUGGA | 194 |
| GAAGAAGGUGGAGAG | 195 |
| GAAGCACUAUGGGCG | 196 |
| GAAGUGAAUUAUAUA | 197 |
| GAAUUAUAUAAAUAU | 198 |
| GACAAUUAUUGUCUG | 199 |
| GACAAUUGGAGAAGU | 200 |
| GACCCCAACCCACAA | 201 |
| GACCUGGAGGAGGAG | 202 |
| GACGGUACAGGCCAG | 203 |
| GAGAAAAAAGAGCAG | 204 |
| GAGAAGUGAAUUAUA | 205 |
| GAGAGAAAAAAGAGC | 206 |
| GAGCAGCAGGAAGCA | 207 |
| GAGCCUGUGCCUCUU | 208 |
| GAGGAGGAGAUAUGA | 209 |
| GAGGGACAAUUGGAG | 210 |
| GAGUUAGGCAGGGAU | 211 |
| GAUAGUAGGAGGCUU | 212 |
| GAUAUAAUCAGUUUA | 213 |
| GAUCAACAGCUCCUA | 214 |
| GAUGCUAAAGCAUAU | 215 |
| GCAACUCACAGUCUG | 216 |
| GCAAGAGUCCUGGCU | 217 |
| GCAAUGUAUGCCCCU | 218 |
| GCACUAUGGGCGCAG | 219 |
| GCAGCAGGAAGCACU | 220 |
| GCAGGAAGCACUAUG | 221 |
| GCAGGGAUACUCACC | 222 |
| GCAUCAGAUGCUAAA | 223 |
| GCCAGACAAUUAUUG | 224 |
| GCCUAAAGCCAUGUG | 225 |
| GCCUCUTCAGCUACC | 226 |
| GCCUGUGCCUCUUCA | 227 |
| GCCUGUGUACCCACA | 228 |
| GCUCCAGGCAAGAGU | 229 |
| GCUCUGGAAAACUCA | 230 |
| GCUGACGGUACAGGC | 231 |
| GCUGUGGAAAGAUAC | 232 |
| GCUGUGGUAUAUAAA | 233 |
| GGAAAAAUAACAUGG | 234 |
| GGAAGCACUAUGGGC | 235 |
| GGACAAUUGGAGAAG | 236 |
| GGAGAAGUGAAUUAU | 237 |
| GGAGCAGCAGGAAGC | 238 |
| GGAGCCUGUGCCUCU | 239 |
| GGAGGAGGAGAUAUG | 240 |
| GGAUCAACAGCUCCU | 241 |
| GGCAAGAGUCCUGGC | 242 |
| GGCAGGGAUACUCAC | 243 |
| GGCAGUCUAGCAGAA | 244 |
| GGCUGUGGAAAGAUA | 245 |
| GGCUGUGGUAUAUAA | 246 |
| GGGACAAUUGGAGAA | 247 |
| GGGAGCAGCAGGAAG | 248 |
| GGGGACCCGACAGGC | 249 |
| GGGGUACCUGUGUGG | 250 |
| GGGUACCUGUGUGGA | 251 |
| GGGUCACAGUCUAUU | 252 |
| GGGUUCUUGGGAGCA | 253 |
| GGUACAGGCCAGACA | 254 |
| GGUACCUGUGUGGAA | 255 |
| GGUAUAGUGCAACAG | 256 |
| GGUAUAUAAAAAUAU | 257 |
| GGUCACAGUCUAUUA | 258 |
| GGUUCUUGGGAGCAG | 259 |
| GUACAAUGUACACAU | 260 |
| GUACACAUGUAAUUA | 261 |
| GUACAGGCCAGACAA | 262 |
| GUACCCACAGACCCC | 263 |
| GUACCUCUGUGGAAA | 264 |
| GUAUAGUGCAACAGC | 265 |
| GUAUAUAAAAAUAUU | 266 |
| GUAUGCCCCUCCCAU | 267 |
| GUCAAUAACGCUGAC | 268 |
| GUCACAGUCUAUUAU | 269 |
| GUCAGCACAGUACAA | 270 |
| GUCUAUUAUGGGGUA | 271 |
| CUCUGGUAUAGUGCA | 272 |
| GUGAAUUAUAUAAAU | 273 |
| GUGCAUCAGAUGCUA | 274 |
| GUGCCUCUUCAGCUA | 275 |
| GUGGAAAAAUAACAU | 276 |
| GUGGAAAGAUACCUA | 277 |
| GUGGAACUUCUGGGA | 278 |
| GUGGGUCACAGUCUA | 279 |
| CUGGUAUAUAAAAAU | 280 |
| GUGUACCCACAGACC | 281 |
| GUUAGGCAGGGAUAC | 282 |
| GUUCCUUGGGUUCUU | 283 |
| GUUCUUGGGAGCAGC | 284 |
| GUUGCAACUCACAGU | 285 |
| GUUUAUGGGAUCAAA | 286 |
| UAAAAAUAUUCAUAA | 287 |
| UAAAGCCAUGUGUAA | 288 |
| UAAAUAUAAAGUAGU | 289 |
| UAACAUGUGGAAAAA | 290 |
| UAACGCUGACGGUAC | 291 |
| UAAUCAGUUUAUGGG | 292 |
| UAAUGAUAGUAGGAG | 293 |
| UACAAUGUACACAUC | 294 |
| UACAGGCCAGACAAU | 295 |
| UACCACCGCUUGACA | 296 |
| UACCCACAGACCCCA | 297 |
| UACCUAAAGGAUCAA | 298 |
| UACCUGUGUGGAAAG | 299 |
| UAGACUUAGGCAGGG | 300 |
| UAGGACUAGCACCCA | 301 |
| UAGGCAGGGAUACUC | 302 |
| UAUUGCAACAGCAAA | 303 |
| UAGUUGGAGUAAUAA | 304 |
| UAUAAAUAUAAAGUA | 305 |
| UAUAAUCAGUUUAUG | 306 |
| UAUAGUGCAACAGCA | 307 |
| UAUAUAAAUAUAAAG | 308 |
| UAUGAGGGACAAUUG | 309 |
| UAUGCCCCUCCCAUC | 310 |
| UAUGGGGUACCUGUG | 311 |
| UAUUAACAAGAGAUG | 312 |
| UAUUAUGGGGUACCU | 313 |
| UAUUCAUAAUGAUAG | 314 |
| UAUUGUCUGGUAUAG | 315 |
| UAUUUUGUGCAUCAG | 316 |
| UCAACAGCUCCUAGG | 317 |
| UCAAUAACGCUGACG | 318 |
| UCACAGUCUAUUAUG | 319 |
| UCACAGUCUGGGGGA | 320 |
| UCAGACCUGGAGGAG | 321 |
| UCAGAUGCUAAAGCA | 322 |
| UCAGCACAGUACAAU | 323 |
| UCAGUUUAUGGGAUC | 324 |
| UCAUAAUGAUAGUAG | 325 |
| UCCAGGCAAGAGUCC | 326 |
| UCCCAUACAUUAUUG | 327 |
| UCCUGGCUGUGGAAA | 328 |
| UCCUUGGGUUCUUGG | 329 |
| UCUAUUAUGGGGUAC | 330 |
| UCUGGUAUAGUGCAA | 331 |
| UCUUCAGCUACCACC | 332 |
| UCUUGGGAGCAGCAG | 333 |
| UGAAUUAUAUAAAUA | 334 |
| UGACGGUACAGGCCA | 335 |
| UGAGGGACAAUUGGA | 336 |
| UGAUAGUAGGAGGCU | 337 |
| UGCAACUCACAGUCU | 338 |
| UGCAUCAGAUGCUAA | 339 |
| UGCCUCUUCAGCUAC | 340 |
| UGCCUGUGUACCCAC | 341 |
| UGCUCUGGAAAACUC | 342 |
| UGGAAAAAUAACAUG | 343 |
| UGGAAAGAUACCUAA | 344 |
| UGGAACUUCUGGGAC | 345 |
| UGGAGAAGUGAAUUA | 346 |
| UGGAGGAGGAGAUAU | 347 |
| UGGAGUAAUAAAUCU | 348 |
| UGGCAGUCUAGCAGA | 349 |
| UGGCUGUGGAAAGAU | 350 |
| UGGCUGUGGUAUAUA | 351 |
| UGGGAGCAGCAGGAA | 352 |
| UGGGGUACCUGUGUG | 353 |
| UGGGUCACAGUCUAU | 354 |
| UGGUUUCUUGGGAGC | 355 |
| UGGUAUAGUGCAACA | 356 |
| UGGUAUAUAAAAAUA | 357 |
| UGUACACAUGGAAUU | 358 |
| UGUACCCACAGACCC | 359 |
| UGUAUGCCCCUCCCA | 360 |
| UGUCAGCACAGUACA | 361 |
| UGUCUGGUAUAGUGC | 362 |
| UGUGCAUCAGAUGCU | 363 |
| UCUGCCUCUUCAGCU | 364 |
| UGUGGAAAAAUAACA | 365 |
| UGUGGAAAGAUACCU | 366 |
| UGUGGAACUUCUGGG | 367 |
| UGUGGUAUAUAAAAA | 368 |
| UGUGUACCCACAGAC | 369 |
| UGUUCCUUGGGUUCU | 370 |
| UGUUGCAACUCACAG | 371 |
| UUAACAUGUGGAAAA | 372 |
| UUAAGAAUAGUUUUU | 373 |
| UUAGGCAGGGAUACU | 374 |
| UUAUAUAAAUAUAAA | 375 |
| UUAUGGGGUACCUGU | 376 |
| UUAUUCUCUGGUAUA | 377 |
| UUCAGACCUGGAGGA | 378 |
| UUCAUAAUGAUAGUA | 379 |
| UUCCCAUACAUUAUU | 380 |
| UUCCUUGGQUUCUUG | 381 |
| UUCUUGGGAGCAGCA | 382 |
| UUGCAACUCACAGUC | 383 |
| UUGGAGAAGUGAAUU | 384 |
| UUGGAGUAAUAAAUC | 385 |
| UUGGCAGCAGCACGA | 386 |
| UUGGGUUCUUGGGAG | 387 |
| UUGUCUCGUAUAGUG | 388 |
| UUGUGCAUCAGAUGC | 389 |
| UUUAACAUGUGGAAA | 390 |
| UUUAUGGGAUCAAAG | 391 |
| UUUGCUGAGGGCUAU | 392 |
| UUUGUGCAUCAGAUG | 393 |
| UUUUAACAUGUGGAA | 394 |
| UUUUGUGCAUCAGAU | 395 |

TABLE IV

HIV env Target and Hammerhead Sequence

| | | | |
|---|---|---|---|
| Substrate | Seq ID | Hammerhead Ribozyme | Seq ID |

| | | | |
|---|---|---|---|
| AAAAAUA A CAUGGUA | 1 | UACCAUG CUGAUGAGGCCGUUAGGCCGAA UAUUUUU | 505 |
| AAAAAUA U UCAUAAU | 2 | AUUAUGA CUGAUGAGGCCGUUAGGCCGAA UAUUUUU | 506 |
| AAAUAUA A AGUAGUA | 11 | UACUACU CUGAUGAGGCCGUUAGGCCGAA UAUAUUU | 507 |
| AAGCCUA A AGCCAUG | 20 | CAUGGCU CUGAUGAGGCCGUUAGGCCGAA UAGGCUU | 508 |
| AAUAUUC A UAAUGAU | 24 | AUCAUUA CUGAUGAGGCCGUUAGGCCGAA GAAUAUU | 509 |
| AAUGAUA G UAGGAGG | 26 | CCUCCUA CUGAUGAGGCCGUUAGGCCGAA UAUCAUU | 510 |
| AAUUAUA U AAAUAUA | 31 | UAUAUUU CUGAUGAGGCCGUUAGGCCGAA UAUAAUU | 511 |
| ACAAUUA U UGUCUGG | 36 | CCAGACA CUCAUGAGGCCGUUAGGCCGAA UAAUUGU | 512 |
| AGAGUUA G GCAGGGA | 61 | UCCCUGC CUGAUGAGGCCGUUACGCCGAA UAACUCU | 513 |
| AGCACUA U GGGCGCA | 64 | UCCCCCC CUGAUGAGGCCGUUAGGCCGAA UAGUGCU | 514 |
| AGGAGUA G CACCCAC | 72 | UUGGGUG CUGAUGAGGCCGUUAGGCCGAA UACUCCU | 515 |
| AUAAAUA U AAAGUAG | 84 | CUACUUU CUGAUGAGGCCGUUAGGCCGAA UAUUUAU | 516 |
| AUCAGUU U AUGGGAU | 97 | AUCCCAU CUGAUGAGGCCGUUAGGCCCAA AACUGAU | 517 |
| AUUCAUA A UGAUAGU | 110 | ACUAUCA CUGAUGAGGCCGUUAGGCCGAA UAUGAAU | 518 |
| CAAUGUA C ACAUGGA | 121 | UCCAUGU CUGAUGAGGCCGUUAGGCCGAA UACAUUG | 519 |
| CAAUGUA U GCCCCUC | 122 | GAGGGGC CUGAUGAGGCCGUUAGGCCGAA UACAUUG | 520 |
| CACAGUC U AUUAUGG | 127 | CCAUAAU CUGAUGAGGCCGUUAGGCCGAA GACUGUG | 521 |
| CACAGUC U GGGGCAU | 128 | AUGCCCC CUGAUGAGGCCGUUAGGCCGAA GACUGUG | 522 |
| CAGUCUA U UAUGGGG | 142 | CCCCAUA CUGAUGAGGCCGUUAGGCCGAA UAGACUG | 523 |
| CAGUUUA U GGGAUCA | 143 | UGAUCCC CUGAUGAGGCCGUUAGGCCGAA UAAACUG | 524 |
| CCAAUUC C CAUACAU | 149 | AUGUAUG CUGAUGAGGCCGUUAGGCCGAA GAAUUGG | 525 |
| CCUCUUC A OCUACCA | 161 | UGGUAGC CUGAUGAGGCCGUUAGGCCGAA GAAGAGG | 526 |
| CUAUUUU G UGCAUCA | 174 | UGAUGCA CUGAUGAGGCCGUUAGGCCGAA AAAAUAG | 527 |
| CUGUGUA C CCACAGA | 186 | UCUGUGG CUGAUGAGGCCGUUAGGCCGAA UACACAG | 528 |
| GACAAUU A UUGUCUG | 199 | CAGACAA CUGAUGAGGCCGUUACGCCGAA AAUUGUC | 529 |
| GACAAUU G GAGAAGU | 200 | ACUUCUC CUGAUGAGGCCGUUAGGCCGAA AAUUGUC | 530 |
| GACGGUA C AGGCCAG | 203 | CUGGCCU CUGAUGAGGCCGUUAGGCCGAA UACCGUC | 531 |
| GAUAGUA G GAGGCUU | 212 | AAGCCUC CUGAUGAGGCCGUUAGGCCGAA UACUAUC | 532 |
| GAUGCUA A AGCAUAU | 215 | AUAUGCU CUGAUGAGGCCGUUAGGCCGAA UAGCAUC | 533 |
| GCAACUC A CAGUCUG | 216 | CAGACUG CUGAUGAGGCCGUUAGGCCGAA GAGUUGC | 534 |
| GCCUCUU C AGCUACC | 226 | GGUAGCU CUGAUGAGGCCGUUAGGCCGAA AAGAGGC | 535 |
| GGCAGUC U AGCAGAA | 244 | UUCUGCU CUGAUGAGGCCGUUAGGCCGAA GACUGCC | 536 |
| GGUUCUU G GGAGCAG | 259 | CUGCUCC CUGAUGAGGCCGUUAGGCCGAA AAGAACC | 537 |
| GUAUAUA A AAAUAUU | 266 | AAUAUUU CUGAUGAGGCCGUUAGGCCGAA UAUAUAC | 538 |
| GUCAAUA A CGCUGAC | 268 | GUCAGCG CUGAUGAGGCCGUUAGGCCGAA UAUUGAC | 539 |
| GUCUAUU A UGGGGUA | 271 | UACCCCA CUGAUGAGGCCGUUAGGCCGAA AAUAGAC | 540 |
| GUGAAUU A UAUAAAU | 273 | AUUUAUA CUGAUGAGGCCGUUAGGCCGAA AAUUCAC | 541 |
| GUGCAUC A GAUGCUA | 274 | UAGCAUC CUGAUGAGGCCGUUAGGCCGAA GAUGCAC | 542 |
| GUGCCUC U UCAGCUA | 275 | UAGCUGA CUGAUGAGGCCGUUAGGCCGAA GAGGCAC | 543 |
| GUGUGUC A CAGUCUA | 279 | UAGACUG CUGAUGAGGCCGUUAGGCCGAA GACCCAC | 544 |
| GUUCCUU G GGUUCUU | 283 | AAGAACC CUGAUGAGGCCGUUAGGCCGAA AAGGAAC | 545 |
| UAGAGUU A GGCAGGG | 300 | CCCUGCC CUGAUGAGGCCGUUAGGCCGAA AACUCUA | 546 |
| UAUAAUC A GUUUAUG | 306 | CAUAAAC CUGAUGAGGCCGUUAGGCCGAA GAUUAUA | 547 |
| UAUUGUC U GGUAUAG | 315 | CUAUACC CUGAUGAGGCCGUUAGGCCGAA GACAAUA | 548 |
| UCAGUUU A UGGGAUC | 324 | GAUCCCA CUGAUGAGGCCGUUAGGCCGAA AAACUGA | 549 |
| UCCCAUA C AUUAUUG | 327 | CAAUAAU CUGAUGAGGCCGUUAGGCCGAA UAUGGGA | 550 |
| UCUAUUA U GGGGUAC | 330 | GUACCCC CUGAUGAGGCCGUUAGGCCGAA UAAUAGA | 551 |
| UCUGGUA U AGUGCAA | 331 | UUGCACU CUGAUGAGGCCGUUAGGCCGAA UACCAGA | 552 |
| UGAAUUA U AUAAAUA | 334 | UAUUUAU CUGAUGAGGCCGUUAGGCCGAA UAAUUCA | 553 |
| UCGAGUA A UAAAUCU | 348 | ACAUUUA CUGAUGAGGCCGUUAGGCCGAA UACUCCA | 554 |
| UGGGGUA C CUCUGUG | 353 | CACACAG CUGAUGAGGCCGUUAGGCCGAA UACCCCA | 555 |
| UGGGUUC U UGGGAGC | 355 | GCUCCCA CUGAUGAGGCCGUUAGGCCGAA GAACCCA | 556 |
| UGGUAUA G UGCAACA | 356 | UGUUGCA CUGAUGAGGCCGUUAGGCCGAA UAUACCA | 557 |
| UGGUAUA U AAAAAUA | 357 | UAUUUUU CUGAUGAGGCCGUUAGGCCGAA UAUACCA | 558 |
| UGUGGUA U AUAAAAA | 368 | UUUUUAU CUGAUGAGGCCGUUAGGCCGAA UACCACA | 559 |
| UUAUAUA A AUAUAAA | 375 | UUUAUAU CUGAUGAGGCCGUUAGGCCGAA UAUAUAA | 560 |
| UUGGGUU C UUGGGAG | 387 | CUCCCAA CUGAUGAGGCCGUUAGGCCGAA AACCCAA | 561 |

TABLE V

HIV env Target and Inozyme Sequence

| | | | |
|---|---|---|---|
| Substrate | Seq ID | Inozyme | Seq ID |

| | | | |
|---|---|---|---|
| AAAGCCA U GUGUAAA | 8 | UUUACAC CUGAUGAGGCCGUUAGGCCGAA IGGCUUU | 562 |
| AAAGCCU A AAGCCAU | 9 | AUGGCUU CUGAUGAGGCCGUUAGGCCGAA IGGCUUU | 563 |
| AAGCACU A UGGGCGC | 19 | GCGCCCA CUGAUGAGGCCCUUAGGCCGAA IGUGCUU | 564 |
| AAUGGCA G UCUAGCA | 27 | UGCUACA CUGAUGAGGCCGUUAGGCCGAA IGCCAUU | 565 |
| AAUGUCA C CACAGUA | 30 | UACUGUG CUGAUGACGCCGUUAGGCCGAA IGACAUU | 566 |
| AAUUCCC A UACAUUA | 32 | UAAUGUA CUGAUGAGGCCGUUAGGCCGAA IGGAAUU | 567 |
| ACAGACC C CAACCCA | 39 | UGGGUUG CUGAUGACGCCGUUAGGCCGAA IGUCUGU | 568 |
| ACACGCC A GACAAUU | 40 | AAUUGUC CUGAUGAGGCCGUUAGCCCGAA IGCCUGU | 569 |
| ACAGUOC A UUAUGGG | 42 | CCCAUAA CUGAUCAGGCCGUUAGGCCGAA IGACUGU | 570 |
| ACAUGCC U GUGUACC | 43 | GGUACAC CUGAUGAGGCCGUUAGGCCGAA IGCAUGU | 571 |
| ACCCACA G ACCCCAA | 46 | UUGGGGU CUGAUGAGGCCGUUAGGCCGAA IGUGGGU | 572 |
| ACUCACA G UCUGGGG | 52 | CCCCAGA CUGAUGAGGCCCUUAGGCCGAA IGUGAGU | 573 |
| AGACCCC A ACCCACA | 57 | UGUGGOC CUGAUGAGGCCGUUAGGCCGAA IGGGUCU | 574 |
| AGAUGCU A AAGCAUA | 62 | UAUGCUU CUGAUGAGGCCGUUAGGCCGAA IGCAUCU | 575 |
| AGCAGCA G GAAGCAC | 65 | GUGCUUC CUGAUGAGGCCCUUAGGCCGAA IGCUGCU | 576 |
| AGCUCCA G GCAAGAG | 69 | CUCUUGC CUGAUGAGGCCGUUAGGCCGAA IGGAGCU | 577 |
| AGGAUCA A CAGCUCC | 73 | GOACCUG CUGAUGAGGCCGUUAGGCCGAA IGAUCCU | 578 |
| AGGGACA A UCUGAGA | 76 | UCUCCAA CUGAUGAGGCCGUUAGGCCGAA IGUCCCU | 579 |
| AUAAUCA G UUUAUGG | 86 | CCAUAAA CUGAUGAGGCCGUUAGGCCGAA TGAUUAU | 580 |
| AUAUUCA U AAUGAUA | 93 | UAUCAUU CUGAUGAGGCCGUUAGGCCGAA IGAAUAU | 581 |
| AUCAACA C CUCCUAG | 95 | CUAGGAG CUGAUGAGGCCGUUAGGCCGAA IGUUGAU | 582 |
| AUGUACA C AUGGAAU | 103 | AUUCCAU CUGAUGAGGCCGUUAGGCCGAA IGUACAU | 583 |
| AUUAACA A GAGAUGG | 107 | CCAUCUC CUGAUGAGGCCGUUAGGCCGAA IGUUAAU | 584 |
| AUUCCCA U ACAUUAU | 111 | AUAAUGU CUGAUGAGGCCGUUAGGCCGAA IGGGAAU | 585 |
| AUUGUCU G GUAUAGU | 113 | ACUAUAC CUGAUGAGGCCGUUAGGCCGAA IGACAAU | 586 |
| AUUUGCU C AGGGCUA | 114 | UAGCCCU CUGAUGAGGCCGUUAGGCCGAA IGCAAAU | 587 |
| CAAAGCC U AAAGCCA | 118 | UGGCUUU CUGAUGAGGCCGUUAGGCCGAA TGCUUUG | 588 |
| CAACUCA C AGUCUGG | 119 | CCAGACU CUGAUGAGGCCGUUAGGCCGAA IGAGUUG | 589 |
| CAAUUCC C AUACAUU | 123 | AAUGUAU CUGAUGAGGCCGUUAGGCCGAA IGAAUUG | 590 |
| CAGACCC C AACCCAC | 132 | GUGGGUU CUGAUGAGGCCGUUAGGCCGAA IGGUCUG | 591 |
| CAGACCU G GAGGAUG | 133 | CCUCCUC CUGAUGAGGCCGUUAGGCCGAA IGGUCUG | 592 |
| CAGCACA G UACAAUG | 135 | CAUUGUA CUGAUGAGGCCGUUAGGCCGAA IGUCCUG | 593 |
| CAGCUCC A GGCAAGA | 137 | UCUUGCC CUGAUGAGGCCGUUAGGCCGAA IGACCUG | 594 |
| CAGGCCA C ACAAUUA | 140 | UAAUUGU CUGAUGAGGCCGUUAGGCCGAA IGGCCUG | 595 |
| CAGUACA A UGUACAC | 141 | GUGUACA CUGAUGAGGCCGUUAGGCCGAA IGUACUG | 596 |
| CAUGCCU C UGUACCC | 147 | GGGUACA CUGAUGAGGCCGUUAGGCCGAA IGGCAUG | 597 |
| CCAGACA A UUAUUGU | 152 | ACAAUAA CUGAUGAGGCCGUUAGGCCGAA TGUCUGG | 598 |
| CCAGGCA A GAGUCCU | 153 | AGGACUC CUGAUGAGGCCGUUAGGCCGAA IGCCUGG | 599 |
| CCAUACA U UAUUGUG | 154 | CACAAUA CUGAUGAGGCCGUUAGGCCGAA IGUAUGG | 600 |
| CCCAACC C ACAAGAA | 155 | UUCUUGU CUGAUGAGGCCGUUAGGCCGAA IGUUGGG | 601 |
| CCUGGCU C UGGAAAG | 163 | CUUUCCA CUGAUGAGGCCGUUAGGCCGAA IGCCAGG | 602 |
| COGUACA C GCCAGAC | 169 | GUCUGGC CUGAUGAGGCCGUUAGGCCGAA TGUACCG | 603 |
| CUCUUCA C CUACCAC | 178 | GUGGUAG CUGAUGAGGCCGUUAGGCCGAA IGAAGAG | 604 |
| CUGUGCC U CUUCAGC | 183 | GCUGAAG CUGAUGAGGCCGUUAGGCCGAA ICCACAG | 605 |
| GACCCCA A CCCACAA | 201 | UUGUGGG CUGAUGAGGCCGUUAGGCCGAA TGGGGUC | 606 |
| GGAAGCA C UAUGGGC | 235 | GCCCAUA CUGAUGAGGCCGUUAGGCCGAA IGCUUCC | 607 |
| GGAGCCU C UGCCUCU | 239 | AGAGGCA CUGAUGACCCCGUUAGGCCGAA IGGCUCC | 608 |
| GGGAGCA G CAGGAAG | 248 | CUUCCUG CUGAUGAGGCCGUUAGGCCGAA IGCUCCC | 609 |
| GGGGACC C GACAGGC | 249 | GCCUGUC CUGAUGAGGCCGUUAGGCCGAA IGUCOCG | 610 |
| GGGUACC U GUGUGGA | 251 | UCCACAC CUGAUGAGGCCGUUAGGCCGAA IGUACCC | 611 |
| GGGUUCU U GGGAGCA | 253 | UGCUCCC CUGAUGAGGCCGUUAGGCCGAA IGAACCC | 612 |
| GGUACCU G UGUGGAA | 255 | UUCCACA CUGAUGAGGCCGUUAGGCCGAA IGGUACC | 613 |
| GGUCACA G UCUAUUA | 258 | UAAUAGA CUGAUGAGGCCGUUAGGCCGAA IGUGACC | 614 |
| GUACACA U GGAAUUA | 261 | UAAUUCC CUGAUGAGGCCGUUAGGCCGAA IGUGUAC | 615 |
| GUACCCA C AGACCCC | 263 | GGGGUCU CUGAUGAGGCCGUUAGGCCGAA IGUGUAC | 616 |
| GUAUGCC C CUCCCAU | 267 | AUGGGAG CUGAUGAGGCCGUUAGGCCGAA IGCAUAC | 617 |
| GUCAGCA C AGUACAA | 270 | UUGUACU CUGAUGAGGCCGUUAGGCCGAA ICCUGAC | 618 |
| GUGUACC C ACAGACC | 281 | GGUCUGU CUGAUGAGGCCGUUAGGCCGAA IGUACAC | 619 |
| UAAAGCC A UGUGUAA | 288 | UUACACA CUGAUGAGGCCGUUAGGCCGAA IGCUUUA | 620 |
| UAACGCU G ACGGUAC | 291 | GUACCGU CUGAUGAGGCCGUUAGGCCGAA IGCGUUA | 621 |
| UACCACC G CUUGAGA | 296 | UCUCAAG CUGAUGAGGCCGUUAGGCCGAA IGUGGUA | 622 |
| UAGUGCA A CAGCAAA | 303 | UUUGCUG CUGAUGAGGCCGUUAGGCCGAA IGCACUA | 623 |
| UAUGCCC C UCCCAUC | 310 | GAUGGGA CUGAUGAGGCCGUUAGGCCGAA IGGCAUA | 624 |
| UCAGACC U GGAGGAG | 321 | CUCCUCC CUGAUGAGGCCGUUAGGCCGAA IGUCUGA | 625 |
| UGCAACU C ACAGUCU | 338 | AGACUGU CUGAUGAGGCCGUUAGGCCGAA IGUUGCA | 626 |
| UGCAUCA C AUGCUAA | 339 | UUAGCAU CUGAUGAGGCCGUUAGGCCGAA IGAUGCA | 627 |
| UGCCUCU U CACCUAC | 340 | GUAGCUG CUGAUGAGGCCGUUAGGCCGAA IGAGUCA | 628 |
| UGGAACU U CUGGGAC | 345 | GUCCCAG CUGAUGAGGCCGUUAGGCCGAA IGGUCCA | 629 |
| UGGGUCA C AGUCUAU | 354 | AUAGACU CUGAUGAGGCCGUUAGGCCGAA IGACCCA | 630 |
| UGUACCC A CAGACCC | 359 | GGGUCUG CUGAUGAGGCCGUUAGGCCGAA IGGUACA | 631 |
| UGUGCCU C UUCAGCU | 364 | AGCUGAA CUGAUGAGGCCGUUAGGCCGAA IGOCACA | 632 |
| UGUUCCU U GGGUUCU | 370 | AGAACCC CUGAUGAGGCCGUUAGGCCGAA IGGAACA | 633 |
| UGUUGCA A CUCACAG | 371 | CUGUGAG CUGAUGAGGCCGUUAGGCCGAA IGCAACA | 634 |
| UUAGGCA G GGAUACU | 374 | AGUAUCC CUGAUGAGGCCGUUAGGCCGAA IGCCUAA | 635 |
| UUGUGCA U CAGAUGC | 389 | GCAUCUG CUGAUGAGGCCGUUAGGCCGAA IGCACAA | 636 |
| UUUAACA U GUGGAAA | 390 | UUUCCAC CUGAUGAGGCCGUUAGGCCGAA IGUUAAA | 637 |

TABLE VI

HIV env Target and G-cleaver Sequence

| | | | |
|---|---|---|---|
| Substrate | Seq ID | G-Cleaver Ribozyme | Seq ID |

| | | | |
|---|---|---|---|
| AACGCUG A CGGUACA | 14 | UGUACCG UGAUG GCAUGCACUAUGC GCG CAGCGUU | 638 |
| AAUAACG C UGACGGU | 22 | ACCGUCA UGAUG GCAUGCACUAUGC GCG CGUUAUU | 639 |
| ACACAUG C CUGUGUA | 38 | UACACAG UGAUG GCAUGCACUAUGC GUG CAUGUGU | 640 |
| ACCACCG C UUGAGAG | 45 | CUCUCAA UGAUG GCAUGCACUAUGC CCC CGGUGGU | 641 |
| ACCUGUG U GGAAAGA | 49 | UCUUUCC UGAUG GCAUGCACUAUGC CCC CACAGGU | 642 |
| AGAAGUG A AUUAUAU | 55 | AUAUAAU UGAUG GCAUGCACUAUGC GCG CACUUCU | 643 |
| AGCAAUG U AUGCCCC | 63 | GGGGCAU UGAUG CCAUGCACUAUGC GCG CAUUGCU | 644 |
| AUGCCUG U GUACCCA | 100 | UGUGUAC UGAUG GCAUGCACUAUGC CCC CAGGCAU | 645 |
| AUCUAUG C CCCUCCC | 104 | GGGAGGG UGAUG GCAUGCACUAUGC GCG CAUACAU | 646 |
| CAAUUUG C UCAGGGC | 125 | GCCCUCA UGAUG CCAUCCACUAUGC GCG CAAAUUG | 647 |
| CAUAAUG A UAGUAGG | 144 | CCUACUA UGAUG GCAUGCACUAUGC CCC CAUUAUG | 648 |
| CCGCUUC A GAGACUU | 159 | AAGUCUC UGAUG GCAUGCACUAUGC GCG CAACCCG | 649 |
| CUGCCUG U GGAAACA | 181 | UCUUUCC UGAUG GCAUCCACUAUGC CCC CAGCCAG | 650 |
| GAUCCUG U GCCUCUU | 208 | AACAGGC UGAUG GCAUGCACUAUCC CCG CAGGCUC | 651 |
| GCCUCUG C CUCUUCA | 227 | UGAAGAG UCAUG GCAUGCACUAUGC GCG CACAGGC | 652 |
| UCCUGUG U ACCCACA | 228 | UGUGGGU UGAUG GCAUGCACUAUCC GCG CACAGUC | 653 |
| GUACCUG U CUGGAAA | 264 | UGUCCAC UGAUG GCAUGCACUAUGC CCC CAGGUAC | 654 |
| UAACAUG U GCAAAAA | 290 | UUUUUCC UGAUG GCAUGCACUAUGC GCG CAUGUUA | 655 |
| UACAAUC U ACACAUC | 294 | CAUGUGU UGAUG CCAUGCACUAUCC GCG CAUUGUA | 656 |
| UAUAGUG C AACAGCA | 307 | UGCUGUU UCAUG CCAUCCACUAUCC GCG CACUAUA | 657 |
| UAUUUUG U GCAUCAG | 316 | CUGAUCC UGAUG GCAUGCACUAUGC CCC CAAAAUA | 658 |
| UCAGAUG C UAAAGCA | 322 | UGCUUUA UGAUG CCAUGCACUAUCC GCC CAUCUCA | 659 |
| UUUGCUG A GGGCUAU | 392 | AUAGCCC UGAUG CCAUGCACUAUGC CCC CACCAAA | 660 |
| UGGUCUC C AUCAGAU | 395 | AUCUGAU UCAUC GCAUGCACUAUGC CCC CACAAAA | 661 |

TABLE VII

HIV env Target and Zinzyme Sequence

| | | | |
|---|---|---|---|
| Substrate | Seq ID | Zinzyme | Seq ID |

| | | | |
|---|---|---|---|
| AAUAACG C UGACGGU | 22 | ACCGUCA GCCGAAAGGCGAGUGAGGUCU CGUUAUU | 662 |
| AAUAGAG U UAGGCAG | 23 | CUGCCUA GCCGAAAGGCGAGUGAGGUCU CUCUAUU | 663 |
| ACACAUG C CUGUGUA | 38 | UACACAG GCCGAAAGGCGAGUGAGGUCU CAUGUGU | 664 |
| ACCACCG C UUGAGAG | 45 | CUCUCAA GCCGAAAGGCGAGUGAGGUCU CGGUGGU | 665 |
| ACCUGUG U GGAAAGA | 49 | UCUUUCC GCCGAAAGGCGAGUGAGGUCU CACAGGU | 666 |
| AGCAAUG U AUGCCCC | 63 | GGGGCAU CCCGAAAGGCGAGUGAGGUCU CAUUGCU | 667 |
| AGUUAGG C AGGGAUA | 80 | UAUCCCU GCCGAAAGGCGAGUGAGGUCU CCUAACU | 668 |
| AUGAUAG U AGGAGGC | 99 | GCCUCCU GCCGAAAGGCGAGUGAGGUCU CUAUCAU | 669 |
| AUGCCUG U GUACCCA | 100 | UGGGUAC GCCGAAAGGCGAGUGAGGUCU CAGGCAU | 670 |
| AUGGCAG U CUACCAG | 101 | CUCCUAG GCCGAAAGGCGAGUGAGGUCU CUGCCAU | 671 |
| AUGUAUG C CCCUCCC | 104 | GGGAGGG GCCGAAAGGCGAGUGAGGUCU CAUACAU | 672 |
| AUGUCAG C ACAGUAC | 105 | GUACUGU GCCGAAAGGCGAGUGAGGUCU CUGACAU | 673 |
| CAAUUUG C UGAGGGC | 125 | GCCCUCA GCCGAAAGGCGAGUGAGGUCU CAAAUUG | 674 |
| CAGGAAG C ACUAUGG | 138 | CCAUAGU GCCGAAAGGCGAGUGAGGUCU CUUCCUG | 675 |
| CCUAAAG C CAUGUGU | 160 | ACACAUG GCCGAAAGGCGAGUGAGGUCU CUUUAGG | 676 |
| CCUUGGG U UCUUGGG | 166 | CCCAACA GCCGAAAGGCGAGUGAGGUCU CCCAAGG | 677 |
| CUCACAG U CUGGGGC | 175 | GCCCCAG GCCGAAAGGCGAGUGAGGUCU CUGUGAG | 678 |
| CUCCAUG C AAGAGUC | 176 | GACUCUU GCCGAAAGGCGAGUGAGGUCU CCUGGAG | 679 |
| CUGACGG U ACAGGCC | 179 | GGCCUGU GCCGAAAGGCGAGUGAGGUCU CCGUCAG | 680 |
| CUGGCUG U GGAAAGA | 181 | UCUUUCC GCCGAAAGGCGAGUGAGGUCU CAGCCAG | 681 |
| CUUUGAG C CAAUUCC | 191 | GGAAUUG GCCGAAAGGCGAGUGAGGUCU CUCAAAG | 682 |
| GAGCCUG U GCCUCUU | 208 | AAGAGGC GCCGAAAGGCGAGUGAGGUCU CAGGCUC | 683 |
| GCAAGAG U CCUGGCU | 217 | AGCCAGG GCCGAAAGGCGAGUGAGGUCU CUCUUGC | 684 |
| GCCUGUG C CUCUUCA | 227 | UGAAGAG GCCGAAAGGCGAGUGAGGUCU CACAGGC | 685 |
| GCCUGUG U ACCCACA | 228 | UGUGGGU GCCGAAAGGCGAGUGAGGUCU CACAGGC | 686 |
| GCUGUGG U AUAUAAA | 233 | UUUAUAU GCCGAAAGGCGAGUGAGGUCU CCACAGC | 687 |
| GGAGAAG U GAAUUAU | 237 | AUAAUUC GCCGAAAGGCGAGUGAGGUCU CUUCUCC | 688 |
| GGAGCAG C AGGAAGC | 238 | GCUUCCU GCCGAAAGGCGAGUGAGGUCU CUGCUCC | 689 |
| GGUAUAG U GCAACAG | 256 | CUGUUGC GCCGAAAGGCGAGUGAGGUCU CUAUACC | 690 |
| GUACAGG C CAGACAA | 262 | UUGUCUG GCCGAAAGGCGAGUGAGGUCU CCUGUAC | 691 |
| GUACCUG U GUGGAAA | 264 | UUUCCAC GCCGAAAGGCGAGUGAGGUCU CAGGUAC | 692 |
| GUCACAG U CUAUUAU | 269 | AUAAUAG GCCGAAAGGCGAGUGAGGUCU CUGUGAC | 693 |
| UAACAUG U GGAAAAA | 290 | UUUUUCC GCCGAAAGGCGAGUGAGGUCU CAUGUUA | 694 |
| UAAUCAG U UUAUGGG | 292 | CCCAUAA GCCGAAAGGCGAGUGAGGUCU CUGAUUA | 695 |
| UACAAUG U ACACAUG | 294 | CAUGUGU GCCGAAAGGCGAGUGAGGUCU CAUUGUA | 696 |
| UAUAGUG C AACAGCA | 307 | UGCUGUU GCCGAAAGGCGAGUGAGGUCU CACUAUA | 697 |
| UAUGGGG U ACCUGUG | 311 | CACAGGU GCCGAAAGGCGAGUGAGGUCU CCCCAUA | 698 |
| UAUUUUG U GCAUCAG | 316 | CUGAUGC GCCGAAAGGCGAGUGAGGUCU CAAAAUA | 699 |
| UCAACAG C UCCUAGG | 317 | CCUAGGA GCCGAAAGGCGAGUGAGGUCU CUGUUGA | 700 |
| UCAGAUG C UAAAGCA | 322 | UGCUUUA GCCGAAAGGCGAGUGAGGUCU CAUCUGA | 701 |
| UCUUCAG C UACCACC | 332 | GGUGGUA GCCGAAAGGCGAGUGAGGUCU CUGAAGA | 702 |
| UGUCUGG U AUAGUGC | 362 | GCACUAU GCCGAAAGGCGAGUGAGGUCU CCAGACA | 703 |
| UUGGGAG C AGCAGGA | 386 | UCCUGCU GCCGAAAGGCGAGUGAGGUCU CUCCCAA | 704 |
| UUUUGUG C AUCAGAU | 395 | AUCUGAU GCCGAAAGGCGAGUGAGGUCU CACAAAA | 705 |

TABLE VIII

| HIV env Target and DNAzyme Sequence | | | |
|---|---|---|---|
| Substrate | Seq ID | DNAzyme | Seq ID |
| AAAAAUA U UCAUAAU | 2 | ATTATGA GGCTAGCTACAACGA TATTTTT | 706 |
| AAAAGAA U GAACAAG | 3 | CTTGTTC GGCTAGCTACAACGA TTCTTTT | 707 |
| AAAAUAA C AUGGUAG | 4 | CTACCAT GGCTAGCTACAACGA TTATTTT | 708 |
| AAAGCCA U GUGUAAA | 8 | TTTACAC GGCTAGCTACAACGA TGGCTTT | 709 |
| AACAUGA C CUGGAUG | 12 | CATCCAG GGCTAGCTACAACGA TCATGTT | 710 |
| AAGUGAA U UAUAUAA | 21 | TTATATA GGCTAGCTACAACGA TTCACTT | 711 |
| AAUAACG C UGACGGU | 22 | ACCGTCA GGCTAGCTACAACGA CGTTATT | 712 |
| AAUAGAG U UAGGCAG | 23 | CTGCCTA GGCTAGCTACAACGA CTCTATT | 713 |
| AAUUAUA U AAAUAUA | 31 | TATATTT GGCTAGCTACAACGA TATAATT | 714 |
| ACAAUUA U UGUCUGG | 36 | CCAGACA GGCTAGCTACAACGA TAATTGT | 715 |
| ACACAUG C CUGUGUA | 38 | TACACAG GGCTAGCTACAACGA CATGTGT | 716 |
| ACCACCO C UUGAGAG | 45 | CTCTCAA GGCTAGCTACAACGA CGGTGGT | 717 |
| ACCCCAA C CCACAAG | 47 | CTTGTGG GGCTAGCTACAACGA TTGGGGT | 718 |
| ACCUGUG U GGAAAGA | 49 | TCTTTCC GGCTAGCTACAACGA CACAGGT | 719 |
| ACGCUGA C GGUACAG | 50 | CTGTACC GGCTAGCTACAACGA TCAGCGT | 720 |
| AGCAAUG U AUGCCCC | 63 | GGGGCAT GGCTAGCTACAACGA CATTGCT | 721 |
| AGCACUA U GGGCGCA | 64 | TGCGCCC GGCTAGCTACAACGA TAGTGCT | 722 |
| AGUACAA U GUACACA | 77 | TGTGTAC GGCTAGCTACAACGA TTGTACT | 723 |
| AGUUAGG C AGGGAUA | 80 | TATCCCT GGCTAGCTACAACGA CCTAACT | 724 |
| AUAAAAA U AUUCAUA | 83 | TATGAAT GGCTAGCTACAACGA TTTTTAT | 725 |
| AUAAAUA U AAAGUAG | 84 | CTACTTT GGCTAGCTACAACGA TATTTAT | 726 |
| AUAAUGA U AGUAGGA | 87 | TCCTACT GGCTAGCTACAACGA TCATTAT | 727 |
| AUAUAAA U AUAAAGU | 90 | ACTTTAT GGCTAGCTACAACGA TTTATAT | 728 |
| AUAUUCA U AAUGAUA | 93 | TATCATT GGCTAGCTACAACGA TGAATAT | 729 |
| AUGAUAG U AGGAGGC | 99 | GCCTCCT GGCTAGCTACAACGA CTATCAT | 730 |
| AUGCCUG U GUACCCA | 100 | TGGGTAC GGCTAGCTACAACGA CAGGCAT | 731 |
| AUGGCAG U CUAGCAG | 101 | CTGCTAG GGCTAGCTACAACGA CTGCCAT | 732 |
| AUGUACA C AUGGAAU | 103 | ATTCCAT GGCTAGCTACAACGA TGTACAT | 733 |
| AUGUAUG C CCCUCCC | 104 | GGGAGGG GGCTAGCTACAACGA CATACAT | 734 |
| AUGUCAG C ACAGUAC | 105 | GTACTGT GGCTAGCTACAACGA CTGACAT | 735 |
| AUUCCCA U ACAUUAU | 111 | ATAATGT GGCTAGCTACAACGA TGGGAAT | 736 |
| AUUUUAA C AUGUGGA | 115 | TCCACAT GGCTAGCTACAACGA TTAAAAT | 737 |
| CAACUCA C AGUCUGG | 119 | CCAGACT GGCTAGCTACAACGA TGAGTTG | 738 |
| CAAUGUA C ACAUGGA | 121 | TCCATGT GGCTAGCTACAACGA TACATTG | 739 |
| CAAUGUA U GCCCCUC | 122 | GAGGGGC GGCTAGCTACAACGA TACATTG | 740 |
| CAAUUUG C UGAGGGC | 125 | GCCCTCA GGCTAGCTACAACGA CAAATTG | 741 |
| CAGACAA U UAUUGUC | 131 | GACAATA GGCTAGCTACAACGA TTGTCTG | 742 |
| CAGGAAG C ACUAUGG | 138 | CCATAGT GGCTAGCTACAACGA CTTCCTG | 743 |
| CAGUCUA U UAUGGGG | 142 | CCCCATA GGCTAGCTACAACGA TAGACTG | 744 |
| CAGUUUA U GGGAUCA | 143 | TGATCCC GGCTAGCTACAACGA TAAACTG | 745 |
| CAUCAGA U GCUAAAG | 146 | CTTTAGC GGCTAGCTACAACGA TCTGATG | 746 |
| CCACAGA C CCCAACC | 150 | GGTTGGG GGCTAGCTACAACGA TCTGTGG | 747 |
| CCAUACA U UAUUGUG | 154 | CACAATA GGCTAGCTACAACGA TGTATGG | 748 |
| CCUAAAG C CAUGUGU | 160 | ACACATG GGCTAGCTACAACGA CTTTAGG | 749 |
| CCUUGGG U UCUUGGG | 166 | cccaaga ggctagctacaacga cccaagg | 750 |

TABLE IX

HIV env Target and Amberzyme Sequence

| | | | |
|---|---|---|---|
| Substrate | Seq ID | Amberzyme | Seq ID |

| | | | |
|---|---|---|---|
| AACGCUG A CGGUACA | 14 | UGUACCG GGAGGAAACUCC CU UCAAGGACAUCGUCCGGG CAGCGUU | 807 |
| AAUAACG C UGACGGU | 22 | ACCGUCA GGAGGAAACUCC CU UCAAGGACAUCGUCCGGG CGUUAUU | 808 |
| AAUAGAG U UAGGCAG | 23 | CUGCCUA GGAGGAAACUCC CU UCAAGGACAUCGUCCGGG CUCUAUU | 809 |
| ACAAUUG G AGAAGUG | 37 | CACUUCU GGAGGAAACUCC CU UCAAGGACAUCGUCCGGG CAAUUGU | 810 |
| ACACAUG C CUGUGUA | 38 | UACACAG GGAGGAAACUCC CU UCAAGGACAUCGUCCGGG CAUGUGU | 811 |
| ACAUGUG G AAAAAUA | 44 | UAUUUUU GGAGGAAACUCC CU UCAAGGACAUCGUCCGGG CACAUGU | 812 |
| ACCACCG C UUGAGAG | 45 | CUCUCAA GGAGGAAACUCC CU UCAAGGACAUCGUCCGGG CGGUGGU | 813 |
| ACCUGUG U GGAAAGA | 49 | UCLUUCC GGAGGAAACUCC CU UCAAGGACAUCGUCCGGG CACAGGU | 814 |
| AGAAGUG A AUUAUAU | 55 | AUAUAAU GGAGGAAACUCC CU UCAAGGACAUCGUCCGGG CACUUCU | 815 |
| AGACCUG G AGGAGGA | 58 | UCCUCCU GGAGGAAACUCC CU UCAAGGACAUCGUCCGGG CAGGUCU | 816 |
| AGCAAUG U AUGCCCC | 63 | GGGGCAU GGAGGAAACUCC CU UCAAGGACAUCGUCCGGG CAUUGCU | 817 |
| AGGCAAG A GUCCUGG | 74 | CCAGGAC GGAGGAAACUCC CU UCAAGGACAUCGUCCGGG CUUGCCU | 818 |
| AGGCAGG G AUACUCA | 75 | UGAGUAU GGAGGAAACUCC CU UCAAGGACAUCGUCCGGG CCUGCCU | 819 |
| AGUUAGG C AGGGAUA | 80 | UAUCCCU GGAGGAAACUCC CU UCAAGGACAUCGUCCGGG CCUAACU | 820 |
| AUAUGAG G GACAAUU | 92 | AAUUGUC GGAGGAAACUCC CU UCAAGGACAUCGUCCGGG CUCAUAU | 821 |
| AUGAGGG A CAAUUGG | 98 | CCAAUUG GGAGGAAACUCC CU UCAAGGACAUCGUCCGGG CCCUCAU | 822 |
| AUGAUAG U AGGAGGC | 99 | GCCUCCU GGAGGAAACUCC CU UCAAGGACAUCGUCCGGG CUAUCAU | 823 |
| AUGCCUG U GUACCCA | 100 | UGGGUAC GGAGGAAACUCC CU UCAAGGACAUCGUCCGGG CAGGCAU | 824 |
| AUGGCAG U CUAGCAG | 101 | CUGCUAG GGAGGAAACUCC CU UCAAGGACAUCGUCCGGG CUGCCAU | 825 |
| AUGUAUG C CCCUCCC | 104 | GGGAGGG GGAGGAAACUCC CU UCAAGGACAUCGUCCGGG CAUACAU | 826 |
| AUGUCAG C ACAGUAC | 105 | GUACUGU GGAGGAAACUCC CU UCAAGGACAUCGUCCGGG CUGACAU | 827 |
| AUUAUGG G GUACCUG | 109 | CAGGUAC GGAGGAAACUCC CU UCAAGGACAUCGUCCGGG CCAUAAU | 828 |
| AUUGGAG A AGUGAAU | 112 | AUUCACU GGAGGAAACUCC CU UCAAGGACAUCGUCCGGG CUCCAAU | 829 |
| CAAAGAG A AGAGUGG | 117 | CCACUCU GGAGGAAACUCC CU UCAAGGACAUCGUCCGGG CUCUUUG | 830 |
| CAAUUGG A GAAGUGA | 124 | UCACUUC GGAGGAAACUCC CU UCAAGGACAUCGUCCGGG CCAAUUG | 831 |
| CAAUUUG C UGAGGGC | 125 | GCCCUCA GGAGGAAACUCC CU UCAAGGACAUCGUCCGGG CAAAUUG | 832 |
| CACUAUG G GCGCAGC | 130 | GCUGCGC GGAGGAAACUCC CU UCAAGGACAUCGUCCGGG CAUAGUG | 833 |
| CAGCAGG A AGCACUA | 136 | UAGUGCU GGAGGAAACUCC CU UCAAGGACAUCGUCCGGG CCUGCUG | 834 |
| CAGGAAG C ACUAUGG | 138 | CCAUAGU GGAGGAAACUCC CU UCAAGGACAUCGUCCGGG CUUCCUG | 835 |
| CAUAAUG A UAGUAGG | 144 | CCUACUA GGAGGAAACUCC CU UCAAGGACAUCGUCCGGG CAUUAUG | 836 |
| CAUGUGG A AAAAUAA | 148 | UUAUUUU GGAGGAAACUCC CU UCAAGGACAUCGUCCGGG CCACAUG | 837 |
| CCCACAG A CCCCAAC | 156 | GUUGGGG GGAGGAAACUCC CU UCAAGGACAUCGUCCGGG CUGUGGG | 838 |
| CCGCUUG A GAGACUU | 159 | AAGUCUC GGAGGAAACUCC CU UCAAGGACAUCGUCCGGG CAAGCGG | 839 |
| CCUAAAG C CAUGUGU | 160 | ACACAUG GGAGGAAACUCC CU UCAAGGACAUCGUCCGGG CUUUAGG | 840 |
| CCUGGAG G AGGAGAU | 162 | AUCUCCU GGAGGAAACUCC CU UCAAGGACAUCGUCCGGG CUCCAGO | 841 |
| CCUUGGG U UCUUGGG | 166 | CCCAAGA GGAGGAAACUCC CU UCAAGGACAUCGUCCGGG CCCAAGG | 842 |
| CUAAAGG A UCAACAG | 171 | CUGUUGA GGAGGAAACUCC CU UCAAGGACAUCGUCCGGG CCUUUAG | 843 |
| CUAGUUG G AGUAAUA | 172 | UAUUACU GGAGGAAACUCC CU UCAAGGACAUCGUCCGGG CAACUAG | 844 |
| CUCACAG U CUGGGGC | 175 | GCCCCAG GGAGGAAACUCC CU UCAAGGACAUCGUCCGGG CUGUGAG | 845 |
| CUCCAGG C AAGAGUC | 176 | GACUCUU GGAGGAAACUCC CU UCAAGGACAUCGUCCGGG CCUGGAG | 846 |
| CUGACGG U ACAGGCC | 179 | GGCCUGU GGAGGAAACUCC CU UCAAGGACAUCGUCCGGG CCGUCAG | 847 |
| CUGGAGG A GGAGAUA | 180 | UAUCUCC GGAGGAAACUCC CU UCAAGGACAUCGUCCGGG CCUCCAG | 848 |
| CUGGCUG U GGAAAGA | 181 | UCUUUCC GGAGGAAACUCC CU UCAAGGACAUCGUCCGGG CAGCCAG | 849 |
| CUUUGAG C CAAUUCC | 191 | GGAAUUG GGAGGAAACUCC CU UCAAGGACAUCGUCCGGG CUCAAAG | 850 |
| GAAGAAG A AGGUGGA | 194 | UCCACCU GGAGGAAACUCC CU UCAAGGACAUCGUCCGGG CUUCUUC | 851 |
| GAAGAAG C UGGAGAG | 195 | CUCUCCA GGAGGAAACUCC CU UCAAGGACAUCGUCCGGG CUUCUUC | 852 |
| GACCUGG A GGAGGAG | 202 | CUCCUCC GGAGGAAACUCC CU UCAAGGACAUCGUCCGGG CCAGGUC | 853 |
| GAGCCUG U GCCUCUU | 208 | AAGAGGC GGAGGAAACUCC CU UCAAGGACAUCGUCCGGG CAGGCUC | 854 |
| GAGGAGG A GAUAUGA | 209 | UCAUAUC GGAGGAAACUCC CU UCAAGGACAUCGUCCGGG CCUCCUC | 855 |
| GAGUUAG G CAGGGAU | 211 | AUCCCUG GGAGGAAACUCC CU UCAAGGACAUCGUCCGGG CUAACUC | 856 |
| GCAAGAG U CCUGGCU | 217 | AGCCAGG GGAGGAAACUCC CU UCAAGGACAUCGUCCGGG CUCUUGC | 857 |
| GCAGCAG G AAGCACU | 220 | AGUGCUU GGAGGAAACUCC CU UCAAGGACAUCGUCCGGG CUGCUGC | 858 |
| GCAUCAG A UGCUAAA | 223 | UUUAGCA GGAGGAAACUCC CU UCAAGGACAUCGUCCGGG CUGAUGC | 859 |
| GCCUGUG C CUCUUCA | 227 | UGAAGAG GGAGGAAACUCC CU UCAAGGACAUCGUCCGGG CACAGGC | 860 |
| GCCUGUG U ACCCACA | 228 | UGUGGGU GGAGGAAACUCC CU UCAAGGACAUCGUCCGGG CACAGUC | 861 |
| GCUCCAG G CAAGAGU | 229 | ACUCCUG GGAGGAAACUCC CU UCAAGGACAUCGUCCGGG CUGGAGC | 862 |
| GCUCUGG A AAACUCA | 230 | UGAGUUU GGAGGAAACUCC CU UCAAGGACAUCGUCCGGG CCAGAGC | 863 |
| GCUGACG C UACAGGC | 231 | GCCUGUA GGAGGAAACUCC CU UCAAGGACAUCGUCCGGG CGUCAGC | 864 |
| GCUGUGG A AAGAUAC | 232 | GUAUCUU GGAGGAAACUCC CU UCAAGGACAUCGUCCGGG CCACAGC | 865 |
| GCUGUGG U AUAUAAA | 233 | UUUAUAU GGAGGAAACUCC CU UCAAGGACAUCGUCCGGG CCACAGC | 866 |
| GGAGAAG U GAAUUAU | 237 | AUAAUUC GGAGGAAACUCC CU UCAAGGACAUCGUCCGGG CUUCUCC | 867 |
| GGAGCAG C AGGAAGC | 238 | GCUUCCU GGAGGAAACUCC CU UCAAGGACAUCGUCCGGG CUGCUCC | 868 |
| GGAGGAG G AGAUAUG | 240 | CAUAUCU GGAGGAAACUCC CU UCAAGGACAUCGUCCGGG CUCCUCC | 869 |
| GGCAGGG A UACUCAC | 243 | GUGAGUA GGAGGAAACUCC CU UCAAGGACAUCGUCCGGG CCCUGCC | 870 |
| GUCUGUG G AAAGAUA | 245 | UAUCUUU GGAGGAAACUCC CU UCAAGGACAUCGUCCGGG CACAGCC | 871 |
| GUCUGUG G UAUAUAA | 246 | UUAUAUA GGAGGAAACUCC CU UCAAGGACAUCGUCCGGG CACAGCC | 872 |
| GGUACAG G CCAGACA | 254 | UGUCUGG GGAGGAAACUCC CU UCAAGGACAUCGUCCGGG CUGUACC | 873 |
| GGUAUAG U GCAACAG | 256 | CUGUUGC GGAGGAAACUCC CU UCAAGGACAUCGUCCGGG CUAUACC | 874 |
| GUACAGG C CAGACAA | 262 | UUGUCUG GGAGGAAACUCC CU UCAAGGACAUCGUCCGGG CCUGUAC | 875 |
| GUACCUG U GUGGAAA | 264 | UUUCCAC GGAGGAAACUCC CU UCAAGGACAUCGUCCGGG CAGGUAC | 876 |
| GUCACAG U CUAUUAU | 269 | AUAAUAG GGAGGAAACUCC CU UCAAGGACAUCGUCCGGG CUGUGAC | 877 |
| GUUCUUG G GAGCAGC | 284 | GCUGCUC GGAGGAAACUCC CU UCAAGGACAUCGUCCGGG CAAGAAC | 878 |
| GUUUAUG C GAUCAAA | 286 | UUUGAUC GGAGGAAACUCC CU UCAAGGACAUCGUCCGGG CAUAAAC | 879 |
| UAACAUG U GGAAAAA | 290 | UUUUUCC GGAGGAAACUCC CU UCAAGGACAUCGUCCGGG CAUGUUA | 880 |
| UAAUCAG U UUAUGGG | 292 | CCCAUAA GGAGGAAACUCC CU UCAAGGACAUCGUCCGGG CUGAUUA | 881 |
| UACAAUG U ACACAUG | 294 | CAUGUGU GGAGGAAACUCC CU UCAAGGACAUCGUCCGGG CAUUGUA | 882 |
| UAGGCAG C GAUACUC | 302 | GAGUAUC GGAGGAAACUCC CU UCAAGGACAUCGUCCGGG CUGCCUA | 883 |
| UAGUUGG A GUAAUAA | 304 | UUAUUAC GGAGGAAACUCC CU UCAAGGACAUCGUCCGGG CCAACUA | 884 |
| UAUAGUG C AACAGCA | 307 | UGCUGUU GGAGGAAACUCC CU UCAAGCACAUCGUCCGGG CACUAUA | 885 |
| UAUGAGG C ACAAUUG | 309 | CAAUUGU GGAGGAAACUCC CU UCAAGCACAUCGUCCGGG CCUCAUA | 888 |
| UAUGCGG U ACCUGUG | 311 | CACAGGU GGAGGAAACUCC CU UCAAGCACAUCGUCCGGG CCCCAUA | 887 |
| UAUUAUG G GGUACCU | 313 | AGGUACC GGAGGAAACUCC CU UCAAGGACAUCGUCCGGG CAUAAUA | 888 |
| UAUUUUG U GCAUCAG | 316 | CUGAUGC GGAGGAAACUCC CU UCAAGGACAUCGUCCGGG CAAAAUA | 889 |
| UCAACAG C UCCUAGG | 317 | CCUAGGA GGAGGAAACUCC CU UCAAGGACAUCGUCCGGG CUGUUGA | 890 |
| UCAGAUG C UAAAGCA | 322 | UGCUUUA GGAGGAAACUCC CU UCAAGGACAUCGUCCGGG CAUCUGA | 891 |
| UCCUUGG C UUCUUGG | 329 | CCAAGAA GGAGGAAACUCC CU UCAAGGACAUCGUCCGGG CCAAGGA | 892 |
| UCUUCAG C UACCACC | 332 | GGUGGUA GGAGGAAACUCC CU UCAAGGACAUCGUCCGGG CUGAAGA | 893 |
| UCUUGGG A GCAGCAG | 333 | CUGCUGC GGAGGAAACUCC CU UCAAGGACAUCGUCCGGG CCCAAGA | 894 |
| UGCUCUG G AAAACUC | 342 | GAGUUUU GCAGGAAACUCC CU UCAAGGACAUCGUCCGGG CAGACCA | 895 |
| UGGAAAG A UACCUAA | 344 | UUAGGUA GGAGGAAACUCC CU UCAAGGACAUCGUCCGGG CUUUCCA | 896 |
| UGUCUOG U AUAGUGC | 362 | GCACUAU GGAGCAAACUCC CU UCAAGGACAUCGUCCGGG CCAGACA | 897 |
| UUAUGGG G UACCUGU | 376 | ACAGGUA GGAGGAAACUCC CU UCAAGGACAUCGUCCGGG CCCAUAA | 898 |
| UUCCUUG G GUUCUUG | 381 | CAAGAAC GGAGGAAACUCC CU UCAAGGACAUCGUCCGGG CAAGGAA | 899 |
| UUCUUGG C AGCAGCA | 382 | UGCUGCU GGAGGAAACUCC CU UCAAGGACAUCGUCCGGG CCAAGAA | 900 |
| UUGGGAG C AGCAGGA | 386 | UCCUGCU GGAGGAAACUCC CU UCAAGGACAUCGUCCGGG CUCCCAA | 901 |
| UUGUCUG G UAUAGUG | 388 | CACUAUA GGAGGAAACUCC CU UCAAGGACAUCGUCCGGG CAGACAA | 902 |
| UUUAUCG C AUCAAAG | 391 | CUUUGAU CGAGGAAACUCC CU UCAAGGACAUCCUCCGGG CCAUAAA | 903 |
| UUUGCUG A GGGCUAU | 392 | AUAGCCC GGACGAAACUCC CU UCAACGACAUCGUCCGGC CACCAAA | 904 |
| UUUUGUG C AUCAGAU | 395 | AUCUGAU GGAGGAAACUCC CU UCAAGGACAUCGUCCGGG CACAAAA | 905 |

TABLE X

HIY env Target and Antisense Sequence

| | | | |
|---|---|---|---|
| Sequence | Seq ID | Antisense | Seq ID |

| | | | |
|---|---|---|---|
| CAGCAGGAAGCACUAUGGGCG | 396 | CGCCCATAGTGCTTCCTGCTG | 906 |
| AGCAGGAAGCACUAUCGGCGC | 397 | GCGCCCATAGTGCTTCCTGCT | 907 |
| GCAGCAGGAAGCACUAUGGGC | 398 | GCCCATAGTGCTTCCTGCTGC | 908 |
| AGCAGCAGGAAGCACUAUGGG | 399 | CCCATAGTGCTTCCTGCTGCT | 909 |
| GAGCAGCAGCAAGCACUAUGG | 400 | CCATAGTGCTTCCTGCTGCTC | 910 |
| GGAGCACCAGGAAGCACUAUG | 401 | CATAGTGCTTCCTCCTGCTCC | 911 |
| CGCUGACGGUACAGGCCAGAC | 402 | GTCTGGCCTGTACCGTCAGCG | 912 |
| ACAAUUGGAGAAGUGAAUUAU | 403 | ATAATTCACTTCTCCAATTGT | 913 |
| ACGCUGACGGUACAGGCCAGA | 404 | TCTGGCCTGTACCGTCACCGT | 914 |
| AUUUAGGCAGGGAUACUCACC | 405 | GGTGAGTATCCCTGCCTAACT | 915 |
| CAAUUGGAGAAGUGAAUUAUA | 406 | TATAATTCACTTCTCCAATTG | 916 |
| GAGUUAGGCAGGGAUACUCAC | 407 | GTGAGTATCCCTGCCTAACTC | 917 |
| AGAGUUAGGCAGGGAUACUCA | 408 | TGAGTATCCCTGCCTAACTCT | 918 |
| AUUGGAGAAGUGAAUUAUAUA | 409 | TATATAATTCACTTCTCCAAT | 919 |
| AAUUGGAGAAGUGAAUUAUAU | 410 | ATATAATTCACTTCTCCAATT | 920 |
| GACAAUUGGAGAAGUGAATUA | 411 | TAATTCACTTCTCCAATTGTC | 921 |
| UUGCAGAAGUGAAUUAUAUAA | 412 | TTATATAATTCACTTCTCCAA | 922 |
| UAGAGUUAGGCAGCGAUACUC | 413 | GAGTATCCCTGCCTAACTCTA | 923 |
| UGCCUGUGUACCCACAGACCC | 414 | GGCTCTGTGGCTACACAGGCA | 924 |
| AUGCCUGUGUACCCACAGACC | 415 | GGTCTGTGGGTACACAGGCAT | 925 |
| AUAGAGUUAGGCAGGGAUACU | 416 | AGTATCCCTGCCTAACTCTAT | 926 |
| CAUGCCUGUGUACCCACAGAC | 417 | GTCTGTGGGTACACAGGCATG | 927 |
| AAUAGACUUAGGCAGCGAUAC | 418 | GTATCCCTGCCTAACTCTATT | 928 |
| ACACAUGCCUGUGUACCCACA | 419 | TGTGGGTACACAGGCATGTGT | 929 |
| CACAUGCCUGUGUACCCACAG | 420 | CTGTGGGTACACACGCATGTG | 930 |
| ACAUCCCUGUGUACCCACAGA | 421 | TCTGTGGGTACACAGGCATGT | 931 |
| GGACAAUUGGAGAAGUGAAUU | 422 | AATTCACTTCTCCAATTGTCC | 932 |
| AGCAAUCUAUGCCCCUCCCAU | 423 | ATGGGAGGGGCATACATTGCT | 933 |
| GCUGACGGUACAGGCCAGACA | 424 | TGTCTGGCCTGTACCGTCAGC | 934 |
| GCCUGUGUACCCACAGACCCC | 425 | GCGGTCTGTGGGTACACAGGC | 935 |
| UAUUAUGGGGUACCUGUGUGG | 426 | CCACACAGGTACCCCATAATA | 936 |
| GCUCCAGGCAAGAGUCCUGGC | 427 | GCCAGGACTCTTGCCTGGAGC | 937 |
| CAGCUCCAGGCAAGAGUCCUC | 428 | CAGGACTCTTGCCTGGAGCTG | 938 |
| AGCUCCAGGCAAGAGUCCUGG | 429 | CCAGGACTCTTGCCTGGAGCT | 939 |
| CUCCAGGCAAGAGUCCUGGCU | 430 | AGCCAGGACTCTTGCCTGGAG | 940 |
| CCUGUGUACCCACAGACCCCA | 431 | TGGGGTCTGTGGGTACACAGG | 941 |
| CUGACGGUACACGCCAGACAA | 432 | TTGTCTGGCCTGTACCGTCAG | 942 |
| CCAAUUCCCAUACAUUAUUGU | 433 | ACAATAATGTATGGGAATTGG | 943 |
| AUUAUGGGGUACCUGUGUGGA | 434 | TCCACACAGGTACCCCATAAT | 944 |
| UACCCACAGACCCCAACCCAC | 435 | GTGGGTTGGGGTCTGTGGGTA | 945 |
| UGUCUGGUAUAGUGCAACAGC | 436 | GCTGTTGCACTATACCAGACA | 946 |
| CUUGGGAGCAGCAGGAAGCAC | 437 | GTGCTTCCTGCTGCTCCCAAG | 947 |
| UCUUGGGAGCACCACGAACCA | 438 | TGCTTCCTGCTGCTCCCAAGA | 948 |
| GUCUGCUAUAGUGCAACAGCA | 439 | TGCTGTTGCACTATACCAGAC | 949 |
| GUACCCACAGACCCCAACCCA | 440 | TGGGTTGGGGTCTGTGGGTAC | 950 |
| UUCUUCGGAGCAGCAGGAAGC | 441 | GCTTCCTCCTGCTCCCAAGAA | 951 |
| UGACGCUACAGGCCAGACAAU | 442 | ATTGTCTGGCCTGTACCGTCA | 952 |
| UGGCUGUGGUAUAUAAAAAUA | 443 | TATTTTTATATACCACAGCCA | 953 |
| UGUGCCUCUUCAGCUACCACC | 444 | GGTGGTAGCTGAAGAGGCACA | 954 |
| GACGGUACAGGCCAGACAAUU | 445 | AATTGTCTGGCCTGTACCGTC | 955 |
| UGUGUACCCACAGACCCCAAC | 446 | GTTGGGGTCTGTGGGTACACA | 956 |
| UGGGGUACCUGUGUGGAAAGA | 447 | TCTTTCCACACAGGTACCCCA | 957 |
| GUGUACCCACAGACCCCAACC | 448 | GGTTGGGGTCTGTGGGTACAC | 958 |
| UAUGGGGUACCUGUGUGGPAA | 449 | TTTCCACACAGGTACCCCATA | 959 |
| AUGGGGUACCUGUGUGGAAAG | 450 | CTTTCCACACAGGTACCCCAT | 960 |
| GGCUGUGGUAUAUAAAAAUAU | 451 | ATATTTTTATATACCACAGCC | 961 |
| UGUACCCACACACCCCAACCC | 452 | GGGTTGGGGTCTGTGGGTACA | 962 |
| CCCACAGACCCCAACCCACAA | 453 | TTGTGGCTTGGGGTCTGTGGG | 963 |
| CUGUGCCUCUUCAGCUACCAC | 454 | GTGGTAGCTGAAGAGGCACAG | 964 |
| GCUGUGGUAUAUAAAAAUAUU | 455 | AATATTTTTATATACCACAGC | 965 |
| CCACAGACCCCAACCCACAAG | 456 | CTTGTGGGTTGGGGTCTGTGG | 966 |
| GUUCUUGGGAGCAGCAGGAAG | 457 | CTTCCTGCTGCTCCCAAGAAC | 967 |
| ACCCACAGACCCCAACCCACA | 458 | TGTGGGTTGGGGTCTGTCGGT | 968 |
| CACAGACCCCAACCCACAAGA | 459 | TCTTGTGGGTTGGGGTCTGTG | 969 |
| CCUGUGCCUCUUCAGCUACCA | 460 | TGGTAGCTGAAGAGGCACACG | 970 |
| ACAGACCCCAACCCACAAGAA | 461 | TTCTTGTGGGTTGGGGTCTGT | 971 |
| GGUUCUUGGGAGCAGCAGGAA | 462 | TTCCTGCTGCTCCCAAGAACC | 972 |
| GCAACUCACAGUCUGGGGCAU | 463 | ATGCCCCAGACTGTGAGTTGC | 973 |
| UGCAACUCACAGUCUGGGGCA | 464 | TGCCCCAGACTGTGAGTTGCA | 974 |
| GGGUUCUUGGGAGCAGCAGGA | 465 | TCCTGCTGCTCCCAAGAACCC | 975 |
| UUGCAACUCACAGUCUGGGGC | 466 | GCCCCAGACTGTGAGTTGCAA | 976 |
| AUGAGGGACAAUUGGAGAAGU | 467 | ACTTCTCCAATTGTCCCTCAT | 977 |
| UGUUGCAACUCACAGUCUGGG | 468 | CCCAGACTGTGAGTTGCAACA | 978 |
| UGAGGGACAAUUGGAGAAGUG | 469 | CACTTCTCCAATTGTCCCTCA | 979 |
| UGAAUUAUAUAAAUAUAAAGU | 470 | ACTTTATATTTATATAATTCA | 980 |
| GUUGCAACUCACAGUCUGGGG | 471 | CCCCAGACTGTGAGTTGCAAC | 981 |
| UGGAGAAGUGAAUUAUAUAAA | 472 | TTTATATAATTCACTTCTCCA | 982 |
| UUGGGUUCUUGGGAGCAGCAG | 473 | CTGCTGCTCCCAAGAACCCAA | 983 |
| AAAGCCUAAAGCCAUGUGUAA | 474 | TTACACATGGCTTTAGGCTTT | 984 |
| UGGGUUCUUGGGAGCAGCAGG | 475 | CCTGCTGCTCCCAAGAACCCA | 985 |
| GGAGAAGUGAAUUAUAUAAAU | 476 | ATTTATATAATTCACTTCTCC | 986 |
| GAGAAGUGAAUUAUAUAAAUA | 477 | TATTTATATAATTCACTTCTC | 987 |
| AGGGACAAUUGGAGAAGUGAA | 478 | TTCACTTCTCCAATTGTCCCT | 988 |
| AAGUGAAUUAUAUAAAUAUAA | 479 | TTATATTTATATAATTCACTT | 989 |
| GAGGGACAAUUGGAGAAGUGA | 480 | TCACTTCTCCAATTGTCCCTC | 990 |
| AUAUGAGGGACAAUUGGAGAA | 481 | TTCTCCAATTGTCCCTCATAT | 991 |
| AGAAGUGAAUUAUAUAAAUAU | 482 | ATATTTATATAATTCACTTCT | 992 |
| CUGUGUACCCACAGACCCCAA | 483 | TTGGGGTCTCTGCGTACACAG | 993 |
| GAAGUGAAUUAUAUAAAUAUA | 484 | TATATTTATATAATTCACTTC | 994 |
| UACAAUGUACACAUGGAAUUA | 485 | TAATTCCATGTGTACATTGTA | 995 |
| CAGUACAAUGUACACAUGGAA | 486 | TTCCATGTGTACATTGTACTG | 996 |
| AAGCCUAAAGCCAUGUGUAAA | 487 | TTTACACATGGCTTTAGGCTT | 997 |
| CCUUCGGUUCUUGGGAGCAGC | 488 | GCTGCTCCCAAGAACCCAAGG | 998 |
| AGUACAAUGUACACAUCGAAU | 489 | ATTCCATGTGTACATTGTACT | 999 |
| UCAAUAACGCUGACGGUACAG | 490 | CTGTACCGTCAGCGTTATTGA | 1000 |
| ACAUGUGGAAAAAUAACAUGG | 491 | CCATCTTATTTTTCCACATGT | 1001 |
| UCCUUGGGUUCUUGGGAGCAG | 492 | CTGCTCCCAAGAACCCAAGGA | 1002 |
| ACGUUACAGGCCAGACAAUUA | 493 | TAATTGTCTGGCCTGTACCGT | 1003 |
| GUACAAUGUACACAUGGAAUU | 494 | AATTCCATGTGTACATTGTAC | 1004 |
| UUGUCUGGUAUAGUGCAACAG | 495 | CTGTTGCACTATACCAGACAA | 1005 |
| UAAUCAGUUUAUGGGAUCAAA | 496 | TTTGATCCCATAAACTGATTA | 1006 |
| AGUGAAUUAUAUAAAUAUAAA | 497 | TTTATATTTATATAATTCACT | 1007 |
| GGGACAAUUGGAGAACUGAAU | 498 | ATTCACTTCTCCAATTGTCCC | 1008 |
| UUAUGGGGUACCUGUGUGGAA | 499 | TTCCACACAGGTACCCCATAA | 1009 |
| AAGCAAUGUAUGCCCCUCCCA | 500 | TGGGAGGGCCATACATTGCTT | 1010 |
| AAUCAGUUUAUGGGAUCAAAG | 501 | CTTTGATCCCATAAACTGATT | 1011 |
| CAAUAACGCUGACGGUACAGG | 502 | CCTGTACCGTCAGCGTTATTG | 1012 |
| GUGCCUCUUCAGCUACCACCG | 503 | CGGTGGTAGCTGAAGAGGCAC | 1013 |
| GAUAUAAUCAGUUUAUGGCAU | 504 | ATCCCATAAACTGATTATATC | 1014 |

TABLE XI

HIV env Target and siRNA Sequence

| | | | | | |
|---|---|---|---|---|---|
| Sequence | Seq ID | siRNA +strand | Seq ID | siRNA −strand | Seq ID |

| | | | | | |
|---|---|---|---|---|---|
| CAGCAGGAAGCACUAUGGGCG | 396 | CAGCAGGAAGCACUAUGGGCGTT | 1015 | CGCCCAUAGUGCUUCCUGCUGTT | 1124 |
| AGCAGGAAGCACUAUGGGCGC | 397 | AGCAGGAAGCACUAUGGGCGCTT | 1016 | GCGCCCAUAGUGCUUCCUGCUTT | 1125 |
| CCAGCAGGAAGCACUAUGGGC | 398 | GCAGCAGCAAGCACUAUCGGCTT | 1017 | GCCCAUAGUGCUUCCUGCUGCTT | 1126 |
| AGCAGCAGGAAGCACUAUGGG | 399 | AGCAGCAGGAAGCACUAUGGGTT | 1018 | CCCAUAGUGCUUCCUGCUGCUTT | 1127 |
| GAGCAGCAGGAAGCACUAUGG | 400 | GAGCAGCAGGAAGCACUAUGGTT | 1019 | CCAUAGUGCUUCCUGCUGCUCTT | 1128 |
| GGAGCAGCAGGAAGCACUAUG | 401 | GGAGCAGCACGAAGCACUAUGTT | 1020 | CAUAGUGCUUCCUGCUGCUCCTT | 1129 |
| CGCUGACGGUACAGGCCAGAC | 402 | CGCUGACGGUACAGGCCAGACTT | 1021 | GUCUGGCCUGUACCGUCAGCGTT | 1130 |
| ACAAUUGGAGAA5UGAAUUAU | 403 | ACAAUUGGAGAAGUGAAUUAUTT | 1022 | AUAAUUCACUUCUCCAAUUGUTT | 1131 |
| ACGCUGACGGUACAGGCCAGA | 404 | ACGCUGACGGUACAGGCCAGATT | 1023 | UCUGGCCUGUACCGUCAGCGUTT | 1132 |
| AGUUAGGCAGGGAUACUCACC | 405 | AGUUAGGCAGGGAUACUCACCTT | 1024 | GGUGAGUAUCCCUGCCUAACUTT | 1133 |
| CAAUUGGAGAAGUGAAUUAUA | 406 | CAAUUGGAGAAGUGAAUUAUATT | 1025 | UAUAAUUCACUUCUCCAAUUGTT | 1134 |
| GAGUUAGGCAGGGAUACUCAC | 407 | GAGUUAGGCAGGGAUACUCACTT | 1026 | GUGAGUAUCCCUGCCUAACUCTT | 1135 |
| AGAGUUAGGCAGGGAUACUCA | 408 | AGAGUUAGGCAGGGAUACUCATT | 1027 | UGAGUAUCCCUGCCUAACUCUTT | 1136 |
| AUUGGAGAAGUGAAUUAUAUA | 409 | AUUGGAGAAGUGAAUUAUAUATT | 1028 | UAUAUAAUUCACUUCUCCAAUTT | 1137 |
| AAUUGGAGAAGUGAAUUAUAU | 410 | AAUUGGAGAAGUGAAUUAUAUTT | 1029 | AUAUAAUUCACUUCUCCAAUUTT | 1138 |
| GACAAUUGGAGAAGUGAAUUA | 411 | GACAATUUGGAGAAGUGAAUUTT | 1030 | UAAUUCACUUCUCCAAUUGUCTT | 1139 |
| UUGGAGAAGUGAAUUAUAUAA | 412 | UUGGAGAAGUGAAUUAUAUAATT | 1031 | UUAUAUAAUUCACUUCUCCAATT | 1140 |
| UAGAGUUAGGCAGGGAUACUC | 413 | UAGAGUUAGGCAGGGAUACUCTT | 1032 | GAGUAUCCCUGCCUAACUCUATT | 1141 |
| UGCCUGUGUACCCACAGACCC | 414 | UGCCUGUGUACCCACAGACCCTT | 1033 | GGGUCUGUGGGUACACAGGCATT | 1142 |
| AUGCCUGUGUACCCACAGACC | 415 | AUGCCUGUGUACCCACAGACCTT | 1034 | GGUCUGUGGGUACACAGGCAUTT | 1143 |
| AUAGAGUUAGGCAGGGAUACU | 416 | AUAGAGUUAGGCAGGGAUACUTT | 1035 | AGUAUCCCUGCCUAACUCUAUTT | 1144 |
| CAUGCCUGUGUACCCACAGAC | 417 | CAUGCCUGUGUACCCACAGACTT | 1036 | GUCUGUGGGUACACAGGCAUGTT | 1145 |
| AAUAGAGUUAGGCAGGGAUAC | 418 | AAUAGAGUUAGGCAGGGAUACTT | 1037 | GUAUCCCUGCCUAACUCUAUUTT | 1146 |
| ACACAUGCCUGUGUACCCACA | 419 | ACACAUGCCUGUGUACCCACATT | 1038 | UGUGGGUACACAGGCAUGUGUTT | 1147 |
| CACAUGCCUGUGUACCCACAG | 420 | CACAUGCCUGUGUACCCACAGTT | 1039 | CUGUGGGUACACAGGCAUGUGTT | 1148 |
| ACAUGCCUGUGUACCCACAGA | 421 | ACAUGCCUGUGUACCCACAGATT | 1040 | UCUGUGGGUACACAGGCAUGUTT | 1149 |
| GGACAAUUGGAGAAGUGAAUU | 422 | GGACAAUUGGAGAAGUGAAUUTT | 1041 | AAUUCACUUCUCCAAUUGUCCTT | 1150 |
| AGCAAUGUAUGCCCCUCCCAU | 423 | AGCAAUCUAUGCCCCUCCCAUTT | 1042 | AUGGCAGGGGCAUACAUUGCUTT | 1151 |
| GCUGACGGUACAGGCCAGACA | 424 | GCUGACGGUACAGGCCAAACATT | 1043 | UGUCUGGCCUGUACCGUCAGCTT | 1152 |
| GCCUGUGUACCCACAGACCCC | 425 | GCCUGUGUACCCACAGACCCCTT | 1044 | GGGGUCUGUGGGUACACAGGCTT | 1153 |
| UAUUAUGGGGUACCUGUGUGG | 426 | UAUUAUGGGGUACCUGUGUGGTT | 1045 | CCACACAGGUACCCCAUAAUATT | 1154 |
| GCUCCAGGCAAGAGUCCUGGC | 427 | GCUCCAGGCAAGAGUCCUGGCTT | 1046 | GCCAGGACUCUUGCCUGGAGCTT | 1155 |
| CAGCUCCAGGCAAGAGUCCUG | 428 | CAGCUCCAGGCAAGAGUCCUGTT | 1047 | CAGGACUCUUGCCUGGAGCUGTT | 1156 |
| AGCUCCAGGCAAGAGUCCUGG | 429 | AGCUCCAGGCAAGAGUCCUGGTT | 1048 | CCAGGACUCUUGCCUGGAGCUTT | 1157 |
| CUCCAGGCAAGAGUCCUGGCU | 430 | CUCCAGGCAAGAGUCCUGGCUTT | 1049 | AGCCAGGACUCUUGCCUGGAGTT | 1158 |
| CCUGUGUACCCACAGACCCCA | 431 | CCUGUGUACCCACAGACCCCATT | 1050 | UGGGGUCUGUGGGUACACAGGTT | 1159 |
| CUGACGGUACAGGCCAGACAA | 432 | CUGACGGUACAGGCCAGACAATT | 1051 | UUGUCUGGCCUGUACCGUCAGTT | 1160 |
| CCAAUUCCCAUACAUUAUUGU | 433 | CCAAUUCCCAUACAUUAUUGUTT | 1052 | ACAAUAAUGUAUGGGAAUUGGTT | 1161 |
| AUUAUGGGGUACCUGUGUGGA | 434 | AUUAUGGGGUACCUGUGUGGATT | 1053 | UCCACACAGGUACCCCAUAAUTT | 1162 |
| UACCCACAGACCCCAACCCAC | 435 | UACCCACAGACCCCAACCCACTT | 1054 | GUGGGUUGGGGUCUGUGGGUATT | 1163 |
| UGUCUGGUAUAGUGCAACAGC | 436 | UGUCUGGUAUAGUGCAACAGCTT | 1055 | GCUGUUGCACUAUACCAGACATT | 1164 |
| CUUGGGAGCAGCAGGAAGCAC | 437 | CUUGGGAGCAGCAGGAAGCACTT | 1056 | GUGCUUCCUGCUGCUCCCAAGTT | 1165 |
| UCUUGGGAGCAGCAGGAAGCA | 438 | UCUUGGGAGCAGCAGGAAGCATT | 1057 | UGCUUCCUGCUGCUCCCAAGATT | 1166 |
| GUCUGGUAUAGUGCAACAGCA | 439 | GUCUGGUAUAGUGCAACAGCATT | 1058 | UGCUGUUGCACUAUACCAGACTT | 1167 |
| GUACCCACAGACCCCAACCCA | 440 | GUACCCACAGACCCCAACCCATT | 1059 | UGGGUUGCGGUCUGUGGGUACTT | 1168 |
| UUCUUGGGAGCAGCAGGAAGC | 441 | UUCUUGGGAGCAGCAGGAAGCTT | 1060 | GCUUCCUGCUGCUCCCAAGAATT | 1169 |
| UGACGGUACAGGCCAGACAAU | 442 | UGACGGUACAGGCCAGACAAUTT | 1061 | AUUGUCUGGCCUGUACCGUCATT | 1170 |
| UGGCUGUGGUAUAUAAAAAUA | 443 | UGGCUGUGGUAUAUAAAAAUATT | 1062 | UAUUUUUAUAUACCACAGCCATT | 1171 |
| UGUGCCUCUUCAGCUACCACC | 444 | UGUGCCUCUUCAGCUACCACCTT | 1063 | GGUGGUAGCUGAAGAGGCACATT | 1172 |
| GACGGUACAGGCCAGACAAUU | 445 | GACGGUACAGGCCAGACAAUUTT | 1064 | AAUUGUCUGGCCUGUACCGUCTT | 1173 |
| UGUGUACCCACAGACCCCAAC | 446 | UGUGUACCCACAGACCCCAACTT | 1065 | GUUGGGGUCUGUGGGUACACATT | 1174 |
| UGGGGUACCUGUGUGGAAAGA | 447 | UGGGGUACCUGUGUGGAAAGATT | 1066 | UCUUUCCACACAGGUACCCCATT | 1175 |
| GUGUACCCACAGACCCCAACC | 448 | GUGUACCCACAGACCCCAACCTT | 1067 | GGUUGGGGUCUGUGGGUACACTT | 1176 |
| UAUGGGGUACCUGUGUGGAAA | 449 | UAUGGGGUACCUGUGUGGAAATT | 1068 | UUGCCACACAGGUACCCCAUATT | 1177 |
| AUGGGGUACCUGUGUGGAAAG | 450 | AUGGGGUACCUGUGUGGAAAGTT | 1069 | CUUUCCACACAGGUACCCCAUTT | 1178 |
| GGCUGUGGUAUAUAAAAAUAU | 451 | GGCUGUGGUAUAUAAAAAUAUTT | 1070 | AUAUUUUUAUAUACCACAGCCTT | 1179 |
| UGUACCCACAGACCCCAACCC | 452 | UGUACCCACAGACCCCAACCCTT | 1071 | GGGUUGGGGUCUGUGGGUACATT | 1180 |
| CCCACAGACCCCAACCCACAA | 453 | CCCACAGACCCCAACCCACAATT | 1072 | UUGUGGGUUGGGGUCUGUGGGTT | 1181 |
| CUGUGCCUCUUCAGCUACCAC | 454 | CUGUGCCUCUUCAGCUACCACTT | 1073 | GUGGUAGCUGAAGAGGCACAGTT | 1182 |
| GCUGUGGUAUAUAAAAAUAUU | 455 | GCUGUGGUAUAUGAAAAUAUUTT | 1074 | AAUAUUUUUAUAUACCACAGCTT | 1183 |
| CCACAGACCCCAACCCACAAG | 456 | CCACAGACCCCAACCCACAAGTT | 1075 | CUUGUGGGUUGGGGUCUGUGGTT | 1184 |
| GUUCUUGGGAGCAGCAGGAAG | 457 | GUUCUUGGGAGCAGCAGGAAGTT | 1076 | CUUCCUGCUGCUCCCAAGAACTT | 1185 |
| ACCCACAGACCCCAACCCACA | 458 | ACCCACAGACCCCAACCCACATT | 1077 | UGUGGGUUGGGGUCUGUGGGUTT | 1186 |
| CACAGACCCCAACCCACAAGA | 459 | CACAGACCCCAACCCACAAGATT | 1078 | UCUUGUGGGUUGCGGUCUGUGTT | 1187 |
| CCUGUGCCUCUUCAGCUACCA | 460 | CCUGUGCCUCUUCAGCUACCATT | 1079 | UGGUAGCUGAAGAGGCACAGGTT | 1188 |
| ACAGACCCCAACCCACAAGAA | 461 | ACAGACCCCAACCCACAAGAATT | 1080 | UUCUUGUGGGUUGGGGUCUGUTT | 1189 |
| GGUUCUUGGGAGCAGCAGGAA | 462 | GGUUCUUGGGACCAGCAGCAATT | 1081 | UUCCUGCUGCUCCCAAGAACCTT | 1190 |
| GCAACUCACAGUCUGGGGCAU | 463 | GCAACUCACAGUCUGGGGCAUTT | 1082 | AUGCCCCAGACUGUGAGUUGCTT | 1191 |
| UGCAACUCACAGUCUGGGGCA | 464 | UGCAACUCACAGUCUGGGGCATT | 1083 | UGCCCCAGACUGUGAGUUGCATT | 1192 |
| GGGUUCUUGGGAGCAGCAGGA | 465 | GGGUUCUUGGGAGCAGCAGGATT | 1084 | UCCUGCUGCUCCCAAGAACCCTT | 1193 |
| UUGCAACUCACAGUCUGGGGC | 466 | UUGCAACUCACAGUCUGGGGCTT | 1085 | GCCCCAGACUGUGAGUUGCAATT | 1194 |
| AUGAGGGACAAUUGGAGAAGU | 467 | AUGAGGGACAAUUGGAGAAGUTT | 1086 | ACUUCUCCAAUUGUCCCUCAUTT | 1195 |
| UGUUGCAACUCACAGUCUGGG | 468 | UGUUGCAACUCACAGUCUGGGTT | 1087 | CCCAGACUGUGAGUUGCAACATT | 1196 |
| UGAGGGACAAUUCGAGAAGUG | 469 | UGAGGGACAAUUGGAGAAGUGTT | 1088 | CACUUCUCCAAUUGUCCCUCATT | 1197 |
| UGAAUUAUAUAAAUAUAAAGU | 470 | UGAAUUAUAUAAAUAUAAAGUTT | 1089 | ACUUUAUAUUUAUAUAAUUCATT | 1198 |
| GUUGCAACUCACAGUCUGGGG | 471 | GUUGCAACUCACAGUCUGGGGTT | 1090 | CCCCAGACUGUGAGUUGCAACTT | 1199 |
| UGGAGAAGUGAAUUAUAUAAA | 472 | UGGAGAAGUGAAUUAUAUAAATT | 1091 | UUUAUAUAAUUCACUUCUCCATT | 1200 |
| UUGGGUUCUUGGGAGCAGCAG | 473 | UUGGGUUCUUGGGAGCAGCAGTT | 1092 | CUGCUGCUCCCAAGAACCCAATT | 1201 |
| AAAGCCUAAAGCCAUGUGUAA | 474 | AAAGCCUAAAGCCAUGUGUAATT | 1093 | UUACACAUGGCUGUAGGCUUUTT | 1202 |
| UGGGUUCUUGGGAGCAGCAGG | 475 | UGGGUUCUUGGGAGCAGCAGGTT | 1094 | CCUGCUGCUCCCAAGAACCCATT | 1203 |
| GGAGAAGUGAAUUAUAUAAAU | 476 | GGAGAAGUGAAUUAUAUAAAUTT | 1095 | AUUUAUAUAAUUCACUUCUCCTT | 1204 |
| GAGAAGUGAAUUAUAUAAAUA | 477 | GAGAAGUGAAUUAUAUAAAUATT | 1096 | UAUUUAUAUAAUUCACUUCUCTT | 1205 |
| AGGGACAAUUGGAGAAGUGAA | 478 | AGGGACAAUUGGAGAAGUGAATT | 1097 | UUCACUUCUCCAAUUGUCCCUTT | 1206 |
| AAGUGAAUUAUAUAAAUAUAA | 479 | AAGUGAAUUAUAUAAAUAUAATT | 1098 | UUAUAUUUAUAUAAUUCACUUTT | 1207 |
| GAGGGACAAUUGGAGAAGUGA | 480 | GAGGGACAAUUGGAGAAGUGATT | 1099 | UCACUUCUCCAAUUGUCCCUCTT | 1208 |
| AUAUGAGGGACAAUUGGAGAA | 481 | AUAUGAGGGACAAUUGGAGAATT | 1100 | UUCUCCAAUUGUCCCUCAUAUTT | 1209 |
| AGAAGUGAAUUAUAUAAAUAU | 482 | AGAAGUGAAUUAUAUAAAUAUTT | 1101 | AUAUUUAUAUAAUUCACUUCUTT | 1210 |
| CUGUGUACCCACAGACCCCAA | 483 | CUGUGUACCCACAGACCCCAATT | 1102 | UUGGGGUCUGUGGGUACACAGTT | 1211 |
| GAAGUGAAUUAUAUAAAUAUA | 484 | GAAGUGAAUUAUAUAAAUAUATT | 1103 | UAUAUUUAUAUAAUUCACUUCTT | 1212 |
| UACAAUGUACACAUGGAAUUA | 485 | UACAAUGUACACAUGGAAUUATT | 1104 | UAAUUCCAUGUGUACAUUGUATT | 1213 |
| CAGUACAAUGUACACAUGGAA | 486 | CAGUACAAUGUACACAUGGAATT | 1105 | UUCCAUGUGUACAUUGUACUGTT | 1214 |
| AAGCCUAAAGCCAUGUGUAAA | 487 | AAGCCUAAAGCCAUGUGUAAATT | 1106 | UUUACACAUGGCUUUAGGCUUTT | 1215 |
| CCUUGGGUUCUUGGGAGCAGC | 488 | CCUUGGGUUCUUGGGAGCAGCTT | 1107 | GCUGCUCCCAAGAACCCAAGGTT | 1216 |
| AGUACAAUGUACACAUGGAAU | 489 | AGUACAAUGUACACAUGGAAUTT | 1108 | AUUCCAUGUGUACAUUGUACUTT | 1217 |
| UCAAUAACGCUGACGGUACAG | 490 | UCAAUAACGCUGACGGUACAGTT | 1109 | CUGUACCGUCAGCGUUAUUGATT | 1218 |
| ACAUGUGGAAAAAUAACAUGG | 491 | ACAUGUGGAAAAAUAACAUGGTT | 1110 | CCAUGUUAUUUUUCCACAUGUTT | 1219 |
| UCCUUGGGUUCUUGGGAGCAG | 492 | UCCUUGGGUUCUUGGGAGCAGTT | 1111 | CUGCUCCCAAGAACCCAAGGATT | 1220 |
| ACGGUACAGGCCAGACAAUUA | 493 | ACGGUACAGGCCAGACAAUUATT | 1112 | UAAUUGUCUGGCCUGUACCGUTT | 1221 |
| GUACAAUGUACACAUGGAAUU | 494 | GUACAAUGUACACAUGGAAUUTT | 1113 | AAUUCCAUGUGUACAUUGUACTT | 1222 |
| UUGUCUGGUAUAGUGCAACAG | 495 | UUGUCUGGUAUAGUGCAACAGTT | 1114 | CUGUUGCACUAUACCAGACAATT | 1223 |
| UAAUCAGUUUAUGGGAUCAAA | 496 | UAAUCAGUUUAUGGGAUCAAATT | 1115 | UUUGAUCCCAUAAACUGAUUATT | 1224 |
| AGUGAAUUAUAUAAAUAUAAA | 497 | AGUGAAUUAUAUAAAUAUAAATT | 1116 | UUUAUAUUUAUAUAAUUCACUTT | 1225 |
| GGGACAAUUGGAGAAGUGAAU | 498 | GGGACAAUUGGAGAAGUGAAUTT | 1117 | AUUCACUUCUCCAAUUGUCCCTT | 1226 |
| UUAUGGGGUACCUGUGUGGAA | 499 | UUAUGGGGUACCUGUGUGGAATT | 1118 | UUCCACACAGGUACCCCAUAATT | 1227 |
| AAGCAAUGUAUGCCCCUCCCA | 500 | AAGCAAUGUAUGCCCCUCCCATT | 1119 | UGGGAGGGGCAUACAUUGCUUTT | 1228 |
| AAUCAGUUUAUGGGAUCAAAG | 501 | AAUCAGUUUAUGGGAUCAAAGTT | 1120 | UUUUGAUCCCAUAAACUGAUUTT | 1229 |
| CAAUAACGCUGACCGUACAGG | 502 | CAAUAACGCUGACGGUACAGGTT | 1221 | CCUGUACCGUCAGCGUUAUUGTT | 1230 |
| GUGCCUCUUCAGCUACCACCG | 503 | GUGCCUCUUCAGCUACCACCGTT | 1122 | CGGUGGUAGCUGAAGAGGCACTT | 1231 |
| GAUAUAAUCAGUUUAUGGGAU | 504 | GAUAUAAUCAGLTUAUGGGAUTT | 1123 | AUCCCAUAAACUGAUUAUAUCTT | 1232 |

TABLE XII

HIV gp41 peptide sequences

| | |
|---|---|
| Peptide Sequence | SEQ ID NO: |

| | |
|---|---|
| YTSLIHSLIEESQNQQEKNEQELLELDKWASLWNWF | 1233 |
| NNLLRAIEAQQHLLQLTVWGIKQLQARILAVERYLKDQ | 1234 |

What we claim is:

1. A short interfering RNA (siRNA) molecule that down-regulates expression of a HIV envelope glycoprotein (env) gene by RNA interference.

2. The siRNA molecule of claim 1, wherein said HIV envelope glycoprotein gene encodes sequence comprising Genbank Accession number NC_{—}001802.

3. The siRNA molecule of claim 1, wherein the siRNA molecule comprises sequence complementary to a nucleic acid sequence encoding HIV envelope glycoprotein or a portion thereof.

4. The siRNA molecule of claim 1, wherein said siRNA molecule comprises about 21 nucleotides.

5. The siRNA molecule of claim 1, wherein said siRNA molecule is double stranded.

6. The siRNA molecule of claim 5, wherein each strand of said siRNA molecule comprises about 21 nucleotides.

7. The siRNA molecule of claim 1, wherein said siRNA molecule has anti-fusogenic activity against HIV entry into a cell.

8. The siRNA molecule of claim 1, wherein said siRNA molecule is chemically synthesized.

9. The siRNA molecule of claim 1, wherein said siRNA molecule comprises at least one nucleic acid sugar modification.

10. The siRNA molecule of claim 1, wherein said siRNA molecule comprises at least one nucleic acid base modification.

11. The siRNA molecule of claim 1, wherein said siRNA molecule comprises at least one nucleic acid backbone modification.

12. A method for modulating HIV cell fusion activity in a cell comprising administering to said cell the siRNA molecule of claim 1 under conditions suitable for modulating said HIV cell fusion activity.

13. The method of claim 12, wherein said cell is a mammalian cell.

14. The method of claim 13, wherein said mammalian cell is a human cell.

15. A method of treating HIV-1 infection in a subject comprising administering to the subject the siRNA of claim 1 under conditions suitable for said treatment.

16. The method of claim 15, wherein said administration is in the presence of a delivery reagent.

17. The method of claim 16, wherein said delivery reagent is a lipid.

18. The method of claim 17, wherein said lipid is a cationic lipid.

19. The method of claim 16, wherein said delivery reagent is a liposome.

20. A composition comprising the siRNA of claim 1 and a pharmaceutically acceptable carrier or diluent.
